(19) United States
(12) Patent Application Publication
(10) Pub. No.: US 2026/0234472 A1
(43) Pub. Date: Aug. 13, 2026

(54) REACTIVE MESOGENS WITH HIGH BIREFRINGENCE

(71) Applicant: MERCK PATENT GMBH, Darmstadt (DE)

(72) Inventors: Kevin ADLEM, FELTHAM, Bedfordshire (GB); James ALLEN, FELTHAM, Bedfordshire (GB); Sarabjot KAUR, FELTHAM, Bedfordshire (GB); Thomas BANKS, UXBRIDGE (GB)

(73) Assignee: MERCK PATENT GMBH, Darmstadt (DE)

(21) Appl. No.: 19/470,787

(22) PCT Filed: Mar. 28, 2024
(86) PCT No.: PCT/EP2024/058443

§ 371 Date: Sep. 29, 2025

(30) Foreign Application Priority Data
Mar. 31, 2023 (EP) 23165889.9
May 17, 2023 (EP) 23173814.7
May 17, 2023 (EP) 23173815.4
Jun. 20, 2023 (EP) 23180281.0
Nov. 20, 2023 (EP) 23210992.6

(51) Int. Cl. C09K 19/38; C07C 69/54; C08F 222/20

(52) U.S. Cl. CPC C09K 19/3861; C07C 69/54; C08F 222/205

(57) ABSTRACT

Reactive mesogens (RMs) with high birefringence and low absorption, RM mixtures and formulations comprising them (as a subcategory of liquid crystal material), polymers and polymer films obtained from such RMs and RM mixtures, and the use of the RMs, RM mixtures, formulations, polymers and polymer films in optical or electrooptical components or devices, especially for digital optics or augmented reality or virtual reality (AR/VR) applications like polarizers, optical compensators, reflective films, diffraction or surface gratings, Bragg polarization gratings (Bragg PG), polarization volume gratings (PVG), polarization volume holograms (PVH), Pancharatnam Berry (PB) gratings, optical waveguides, lenses or PB lenses.

### CROSS-REFERENCE TO RELATED APPLICATIONS

This application is the United States National Phase under 35 U.S.C. § 371 of PCT International Patent Application No. PCT/EP2024/058443, filed on Mar. 28, 2024, which claims the benefit of European Patent Application No. 23210992.6 filed on Nov. 20, 2023, European Patent Application No. 23180281.0 filed on Jun. 20, 2023, European Patent Application No. 23173815.4, filed on May 17, 2023, European Patent Application No. 23173814.7, filed on May 17, 2023, and European Patent Application No. 23165889.9, filed on Mar. 31, 2023, the disclosures of which are hereby incorporated by reference herein in their entireties.

### FIELD OF THE INVENTION

The invention relates to reactive mesogens (RMs) with high birefringence and low absorption, to RM mixtures and formulations comprising them (as a subcategory of liquid crystal material), to polymers and polymer films obtained from such RMs and RM mixtures, and the use of the RMs, RM mixtures, formulations, polymers and polymer films in optical or electrooptical components or devices, especially for digital optics or augmented reality or virtual reality (AR/VR) applications like polarizers, optical compensators, reflective films, diffraction or surface gratings, Bragg polarization gratings (Bragg PG), polarization volume gratings (PVG), polarization volume holograms (PVH), Pancharatnam Berry (PB) gratings, optical waveguides, lenses or PB lenses.

### BACKGROUND AND PRIOR ART

Reactive mesogens (RMs), mixtures or formulations comprising them, and LC polymer (LCP) films obtained thereof, can be used to make optical components, like compensation, retardation or polarisation films, or lenses. These optical components can be used in optical or electrooptical devices like LC displays. Usually the RMs or RM mixtures are polymerised through the process of in-situ polymerisation.

The availability of RM materials and LCP films with a high birefringence is of high importance for manufacturing optical components of modern display devices like LCDs, but also in the newly emerging area of digital optics, including augmented reality or virtual reality (AR/VR) applications, where the LCP films can be used for example as optical waveguides. These applications require materials which exhibit a high average refractive index (I) while maintaining high birefringence (Δn). RM materials and LCP films have been proposed for example to produce diffraction gratings such as Pancharatnam-Berry gratings (PBG) and lenses, also referred to as Bragg polarization gratings (Bragg PG) or polarization volume gratings (PVG), but also for polarization volume holograms (PVH). The best alignment for RMs in PVGs is typically achieved for chiral nematic (hereinafter also referred to as “cholesteric”) systems in which the chiral structure promotes the self-organization of the bulk LC.

To maximise the performance of the optical element, multiple properties of the cholesteric RM material and LCP film, like the cholesteric pitch, the optical indices (extraordinary (nₑ), ordinary (nₒ) and average (n̅)), the birefringence Dn and the film thickness (d) must be designed and carefully controlled. It is also sometimes required to vary these properties within one optical element, for example when using multiple LCP layers with different pitch, thickness, refractive indices and/or slant angle.

Moreover, when producing multilayer optical elements where multiple RM layers are coated directly onto each other, it is important to ensure good and uniform alignment quality, prevent the underlying RM layer from damage when coating the next RM layer on top of it, and enable strong intermolecular interactions between the RM layers to impart the alignment direction from one RM layer to the next RM layer.

For some optical components of LCDs or AR/VR devices, the manufacture of cholesteric LCP films having a broad reflection bandwidth and a high birefringence is desired.

RMs with high birefringence are known in prior art, and are for example described in WO 2022/233908 A1. However, as the birefringence of the RMs increases, generally their absorbance of visible light also increases, which can lead to undesired colouring. On the other hand, with the increasing need for high birefringence materials to generate improved optical effects in LCP films, the need for optical purity of these materials is a key factor. Also, for specific uses RM materials are preferred which have a high extraordinary refractive index nₑ and/or a low or moderate ordinary refractive index nₒ. To satisfy these needs, usually RM materials were designed with a value nₑ as high as possible. Although the relationship between light absorbance and nₑ of the RM materials is still complicated and not easily predictable, some trends are generally in place. Thus, normally the light absorbance at increasing wavelengths is closely related to the nₑ value. This means that, as nₑ increases, both the absorbance peak and the cut-off point at zero absorbance are shifted to higher wavelengths.

It is therefore desirable to provide RM materials, including RMs and mixtures comprising them, for use in polymer films, especially for use in digital optics, which show a high birefringence, especially a high nₑ value, but at the same time do not show strong colouring and high light absorbance. The RM materials should be easily processable for the production of LCP films, allow easy and precise control of optical properties like the birefringence, average refractive index and light absorbance, and enable good alignment quality also in multilayer systems.

It is an aim of the present invention to provide RM materials and LCP films which show one or more of the above-mentioned advantages. Other aims of the present invention are immediately evident to the person skilled in the art from the following detailed description.

Surprisingly, the inventors of the present invention have found that these aims could be achieved by providing RMs and RM mixtures according to the invention as described and claimed hereinafter.

### SUMMARY OF THE INVENTION

The present invention relates to a polymerisable mesogenic compound, hereinafter also referred to as “reactive mesogen” or “RM”, of formula I,

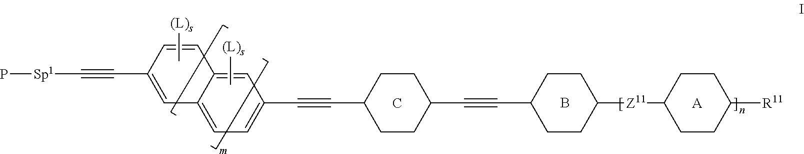

wherein the individual radicals, independently of each other and on each occurrence P identically or differently, have the following meanings
P a polymerisable group,
Sp a spacer group or a single bond,
Sp¹ a spacer group or a single bond, preferably alkylene with 1 to 12, more preferably with 3 to 6, C atoms,
R¹¹ H, F, Cl, CN, CH═CH₂, alkyl, alkoxy, thioalkyl, alkylcarbonyl, alkoxycarbonyl, alkylcarbonyloxy or alkoxycarbonyloxy with 1 to 6, preferably with 1 to 3, C atoms which is optionally fluorinated or chlorinated, or P-Sp,
A, B phenylene-1,4-diyl, naphthalene-1,4-diyl, naphthalene-2,6-diyl, fluorene-2,7-diyl, dibenzothiophene-2,7-diyl, dibenzofuran-2,7-diyl, benzo[1,2-b:4,5-b′]dithiophene-2,5-diyl, indole-4,7-diyl, benzothiophene-4,7-diyl, 9,10-dihydro-phenanthrene-2,7-diyl or 1,2,3,4-tetrahydronaphthalene-5,8-diyl where, in addition, one or more CH groups in these groups may be replaced by N, all of which are optionally substituted by one or more groups L or P-Sp-.
C phenylene-1,4-diyl, naphthalene-1,4-diyl, or naphthalene-2,6-diyl, preferably phenylene-1,4-diyl or naphthalene-2,6-diyl, in which, in addition, one or more CH groups in these groups may be replaced by N, and which are optionally substituted by one or more groups L or P-Sp-,
   provided that not more than one of A, B and C may denote naphthalene-1,4-diyl,
L F, Cl, —CN, —SCN, P-Sp-, or straight chain, branched or cyclic alkyl having 1 to 25 C atoms, wherein one or more non-adjacent CH₂-groups are optionally replaced by —O—, —S—, —CO—, —CO—O—, —O—CO—, —O—CO—O—, CR⁰═CR⁰⁰—, —C≡C—,

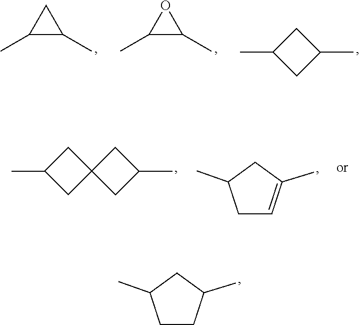

in such a manner that O- and/or S-atoms are not directly connected with each other, and wherein one or more H atoms are each optionally replaced by P-Sp-, F or Cl, or two substituents L that are connected to directly adjacent C atoms may also form a cycloalkyl or cycloalkenyl group with 5, 6, 7 or 8 C atoms,
Z¹¹ —O—, —S—, —CO—, —COO—, —OCO—, —S—CO—, —CO—S—, —O—COO—, —CO—NR⁰—, —NR⁰—CO—, —NR⁰—CO—NR⁰⁰, —NR⁰—CO—O—, —O—CO—NR⁰—, —OCH₂—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CR⁰—, —CY¹═CY²—, —C≡C—, —CH═CH—COO—, —OCO—CH═CH— or a single bond, preferably —COO—, —OCO—, —C≡C—, or a single bond, most preferably a single bond,
n1 1, 2, 3 or 4,
s 0, 1, 2 or 3, preferably 0, 1 or 2,
R⁰, R⁰⁰ H or alkyl having 1 to 12 C atoms,
Y¹, Y² H, F, Cl, NCS, or CN,
m 0 or 1,
n 0, 1, 2, 3 or 4, preferably 0, 1 or 2, more preferably 0 or 1, most preferably 0.

The invention further relates to a mixture, which is hereinafter referred to as “RM mixture”, comprising one or more compounds of formula I, and optionally further comprising one or more reactive mesogens, preferably selected from mono- and direactive mesogens, which are different from formula I, and optionally further comprising one or more chiral compounds which are optionally polymerisable and/or isomerisable, and optionally further comprising one or more additives.

The invention further relates to a formulation, which is hereinafter referred to as “RM formulation”, comprising one or more compounds of formula I or comprising an RM mixture as described above and below, and further comprising one or more solvents and/or additives.

The invention further relates to a polymer film obtainable or obtained by polymerising one or more compounds of formula I, or an RM mixture or RM formulation as described above and below and to a process of its preparation, preferably wherein the RMs are aligned, and preferably at a temperature where the RMs or RM mixture exhibit a liquid crystal phase.

The invention further relates to the use of the compounds of formula I or the RM mixture or the polymer film as described above and below in optical, electrooptical or electronic components or devices.

The invention further relates to an optical, electrooptical or electronic device or a component comprising a compound of formula I or an RM mixture or polymer film as described above and below.

Said components include, without limitation, optical retardation films, polarizers, optical compensators, reflective films, diffraction or surface gratings such as Bragg polarization gratings (Bragg PG), polarization volume gratings (PVG), polarization volume holograms (PVH), Pancharatnam Berry (PB) gratings, furthermore nonmechanical beam steering elements, optical waveguides, optical couplers or combiners, polarization beam splitters, partial mirrors, reflective films, alignment layers, colour filters, antistatic protection sheets, electromagnetic interference protection sheets, lenses for light guides, focusing and optical effects, polarization controlled lenses, PB lenses and IR reflection films; for example for use in LC displays (LCDs), organic light emitting diodes (OLEDs), autostereoscopic 3D displays, see-through near-eye displays, augmented reality(AR) or virtual reality (VR) systems, switchable windows, spatial light modulators, optical data storage, remote optical sensing, holography, spectroscopy, optical telecommunications, polarimetry or front/back-lighting.

Said devices include, without limitation, electro optical displays, especially LCDs, OLEDs, autostereoscopic 3D displays, see-through near-eye displays, AR/VR systems, goggles for AR/VR applications, switchable windows, spatial light modulators, optical data storage devices, optical sensors, holographic devices, spectrometers, optical telecommunication systems, polarimeters or front-/backlights.

### DETAILED DESCRIPTION OF THE DRAWINGS

The patent or application file contains at least one drawing executed in color. Copies of this patent or patent application publication with color drawing(s) will be provided by the Office upon request and payment of the necessary fee.

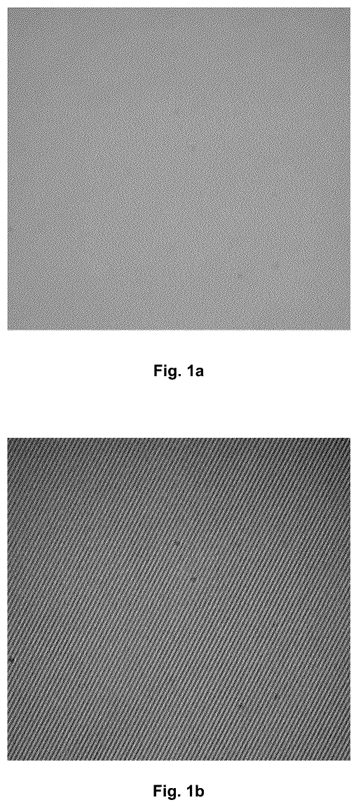

FIG. 1a (×20 magnification) shows a microscopic image of a PVH grating coated with a first polymerised RM layer L1 according to Example 5.

FIG. 1b (×100 magnification) shows a microscopic image of a PVH grating coated with a first polymerised RM layer L1 according to Example 5.

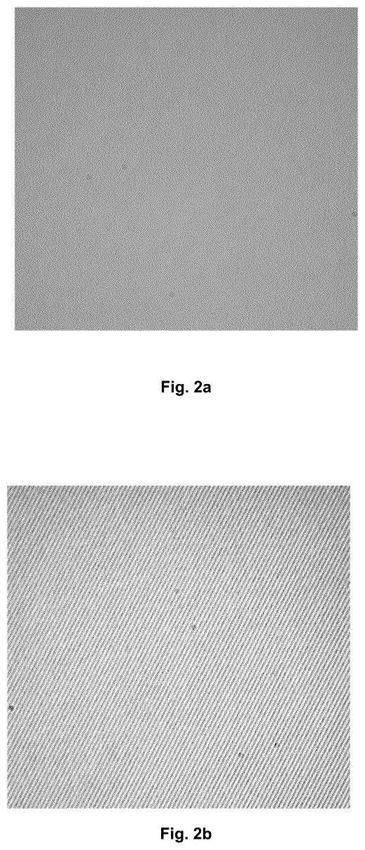

FIG. 2a (×20 magnification) shows a microscopic image of a PVH grating coated with a first polymerised RM layer L1 and a second polymerised RM layer L2 according to Example 5.

FIG. 2b (×100 magnification) shows a microscopic image of a PVH grating coated with a first polymerised RM layer L1 and a second polymerised RM layer L2 according to Example 5.

### DEFINITIONS OF TERMS

Throughout the description and claims of this specification, the words “comprise” and “contain” and variations of the words, for example “comprising” and “comprises”, mean “including but not limited to”, and are not intended to (and do not) exclude other components.

Unless the context clearly indicates otherwise, as used herein plural forms of the terms herein are to be construed as including the singular form and vice versa.

The term “film” as used herein includes rigid or flexible, self-supporting or free-standing films with mechanical stability, as well as coatings or layers on a supporting substrate or between two substrates.

The term “per- and/or polyfluoroalkyl substance (PFAS)” as used herein (following the definition by the OECD) means a substance or compound that contains at least one fully fluorinated methyl or methylene C atom (without any H/Cl/Br/I atom attached to it), i.e., a compound with at least one CF₃ or CF₂ group.

The expression “polyfluorinated alkyl or aryl group” as used herein means an alkyl or aryl group which is substituted by two or more F atoms (wherein the F atoms may be attached either to the same or different C atoms), thus including perfluorocarbon groups.

As used herein, the terms “reactive mesogen” and “RM” will be understood to mean a compound containing a mesogenic or liquid crystalline skeleton, and one or more functional groups attached thereto, optionally via spacer groups, which are suitable for polymerisation and are also referred to as “polymerisable group” or “P”.

Unless stated otherwise, the term “polymerisable compound” as used herein will be understood to mean a polymerisable monomeric compound.

Polymerisable compounds or RMs with one polymerisable group are also referred to as “monoreactive” compounds, polymerisable compounds or RMs with two polymerisable groups as “direactive” compounds, and polymerisable compounds or RMs with more than two polymerisable groups as “multireactive” compounds. Compounds without a polymerisable group are also referred to as “non-reactive” compounds.

The terms “liquid crystal”, “mesogen” and “mesogenic compound” as used herein mean a compound that under suitable conditions of temperature, pressure and concentration can exist as a mesophase or in particular as a LC phase.

The term “clearing point” means the temperature at which the transition between the mesophase with the highest temperature range and the isotropic phase occurs.

The term “mesogenic group” as used herein is known to the person skilled in the art and described in the literature, and means a group which, due to the anisotropy of its attracting and repelling interactions, essentially contributes to causing a liquid-crystal (LC) phase in low-molecular-weight or polymeric substances. Compounds containing mesogenic groups (mesogenic compounds) do not necessarily have to have an LC phase themselves. It is also possible for mesogenic compounds to exhibit LC phase behaviour only after mixing with other compounds and/or after polymerisation. Typical mesogenic groups are, for example, rigid rod- or disc-shaped units. An overview of the terms and definitions used in connection with mesogenic or LC compounds is given in Pure Appl. Chem. 2001, 73(5), 888 and C. Tschierske, G. Pelzl, S. Diele, Angew. Chem. 2004, 116, 6340-6368.

The term “spacer group”, hereinafter also referred to as “Sp”, as used herein is known to the person skilled in the art and is described in the literature, see, for example, Pure Appl. Chem. 2001, 73(5), 888 and C. Tschierske, G. Pelzl, S. Diele, Angew. Chem. 2004, 116, 6340-6368. As used herein, the terms “spacer group” or “spacer” mean a flexible group, for example an alkylene group, which connects the mesogenic group and the polymerisable group(s) in a polymerisable mesogenic compound.

As used herein, the term “RM mixture” means a mixture comprising one or more, preferably two or more, more preferably two to ten, very preferably two to six RMs, The RM mixture optionally further comprises one or more solid additives including, without being limited to, polymerisation initiators, inhibitors, surfactants and adhesion promoters, etc. as described in more detail below.

As used herein, the term “RM formulation” means at least one RM or RM mixture, and one or more other materials added to the at least one RM or RM mixture to provide, or to modify, specific properties of the RM formulation and/or of the at least one RM therein. It will be understood that an RM formulation is also a vehicle for carrying the RM to a substrate to enable the forming of layers or structures thereon. Exemplary materials include, but are not limited to, solvents, polymerisation initiators, inhibitors, surfactants and adhesion promoters, etc. as described in more detail below.

Unless stated otherwise, the percentage of a compound in an RM mixture as given above and below means % by weight of all solids in the RM mixture, excluding any solvents or liquid additives that may be present.

Unless stated otherwise, the percentage of a compound in an RM formulation as given above and below means % by weight of all solids in the RM formulation, including liquid additives as described below but excluding solvents.

As used herein, the term “polymer” will be understood to mean a molecule that encompasses a backbone of one or more distinct types of repeating units (the smallest constitutional unit of the molecule) and is inclusive of the commonly known terms “oligomer”, “copolymer”, “homopolymer” and the like. Further, it will be understood that the term polymer is inclusive of, in addition to the polymer itself, residues from initiators, catalysts, and other elements attendant to the synthesis of such a polymer, where such residues are understood as not being covalently incorporated thereto. Further, such residues and other elements, while normally removed during post polymerisation purification processes, are typically mixed or co-mingled with the polymer such that they generally remain with the polymer when it is transferred between vessels or between solvents or dispersion media.

The term “polymerisation” means the chemical process to form a polymer by bonding together multiple polymerisable groups or polymer precursors (polymerisable compounds) containing such polymerisable groups.

A “polymer network” is a network in which all polymer chains are interconnected to form a single macroscopic entity by many crosslinks.

The polymer network can occur in the following types:
A graft polymer molecule is a branched polymer molecule in which one or more the side chains are different, structurally or configurationally, from the main chain.
A star polymer molecule is a branched polymer molecule in which a single branch point gives rise to multiple linear chains or arms. If the arms are identical, the star polymer molecule is said to be regular. If adjacent arms are composed of different repeating subunits, the star polymer molecule is said to be variegated.
A comb polymer molecule consists of a main chain with two or more three-way branch points and linear side chains. If the arms are identical the comb polymer molecule is said to be regular.
A brush polymer molecule consists of a main chain with linear, unbranched side chains and where one or more of the branch points has four-way functionality or larger.

The term “chiral” in general is used to describe an object that is non-superimposable on its mirror image.

“Achiral” (non-chiral) objects are objects that are identical to their mirror image.

The terms “chiral nematic” and “cholesteric” are used synonymously in this application, unless explicitly stated otherwise.

The term “isomerisable/photoisomerisable compound” means a compound comprising one or more isomerisable or photoisomerisable groups, respectively.

The term “isomerisable group” means a functional group of a molecule that causes a change of the geometry of the molecule, i.e. isomerisation, either by bond rotation, skeletal rearrangement or atom- or group-transfer, or by dimerization, which can be induced, e.g., thermally or photochemically or by adding a catalyst.

The term “photoisomerisable group” means a functional group of a molecule that causes a change of the geometry of the molecule, i.e. isomerisation, either by bond rotation, skeletal rearrangement or atom- or group-transfer, or by dimerization, upon irradiation with light of a suitable wavelength that can be absorbed by the molecule (photoisomerisation).

Examples of photoisomerisable groups are —C═C— double bonds and azo groups (—N═N—). Examples of molecular structures and sub-structures comprising such photoisomerisable groups are stilbene, (1,2-difluoro-2-phenyl-vinyl)-benzene, cinnamate, a-cyanocinnamate, 4-phenylbut-3-en-2-one, Schiff base (i.e., a group RⁱRⁱⁱC═NRⁱⁱⁱ, wherein Rⁱⁱⁱ is different from H, and is for example alkyl or aryl), 2-benzyliden-1-indanone, chalcone, coumarin, chromone, pentalenone and azobenzene.

A chiral RM mixture in accordance with the present invention can be prepared, for example, by doping a host mixture comprising one or more RMs with a chiral compound having a high twisting power.

The pitch p (in nm) of the induced cholesteric helix, hereinafter also referred to as “chiral pitch” or “helical pitch” is then given by the concentration c (in %) and the helical twisting power HTP (in nm⁻¹) of the chiral compound in accordance with the following equation:

$$p = {( HTP c )}^{- 1}$$

A low value of the pitch is hereinafter also referred to as “short pitch”, and a high value of the pitch is hereinafter also referred to as “long pitch”. Also, a short pitch corresponds to a highly twisted structure, i.e., a higher twist angle, and a long pitch corresponds to a slowly twisted structure, i.e., a lower twist angle, around the helix axis within a given distance.

The twist angle, θ through a thickness, d, is defined by the following equation:

$$θ = \frac{( 360 * d )}{p}$$

where p is the pitch as defined above.

In case more than one chiral compound is used, the total HTP of the chiral compounds having the same configuration or twist sense (HTPₜₒₜₐₗ) holds then approximately the following equation:

$${HTP}_{total} = {\sum }_{i} {c}_{1} {HTP}_{i}$$

wherein cᵢ is the concentration of each individual chiral compound and HTPᵢ is the helical twisting power of each individual chiral compound.

The HTP of all chiral compounds within a mixture of different configurations or different twist sense (|HTP_{D}|) holds then approximately the following equation:

$$\vert {HTP}_{D} \vert = ( {\sum }_{s} {c}_{s} {HTP}_{s} ) - ( ( {\sum }_{r} {c}_{r} {HTP}_{r} )$$

wherein cₛ is the concentration of each individual chiral compound with S configuration, HTPₛ is the helical twisting power of each individual chiral compound having S configuration and wherein cᵣ is the concentration of each individual chiral compound with R configuration and HTP_{R} is the helical twisting power of each individual chiral compound having R configuration.

The birefringence Δn is defined as follows

$$Δ n = {n}_{e} - {n}_{o}$$

wherein nₑ is the extraordinary refractive index and nₒ is the ordinary refractive index, and the effective average refractive index nₐᵥ, is given by the following equation:

$${n}_{av .} = {( ( 2 {n}_{o}^{2} + {n}_{e}^{2} ) / 3 )}^{1 / 2}$$

The average refractive index nₐᵥ. and the ordinary refractive index nₒ can be measured using an Abbe refractometer. Δn can then be calculated from the above equations.

The central wavelength λ and bandwidth Δλ of a reflectance band of cholesteric RM or LC material or a cholesteric polymer film are given by the pitch p of the cholesteric helix, the average refractive index nₐᵥ. and the birefringence Δn of the cholesteric liquid crystal in accordance with the following equations:

$$λ = {n}_{av .} \cdot p \\ Δ λ = Δ n \cdot p$$

The term “visible light” means electromagnetic radiation with a wavelength in a range from about 400 nm to about 740 nm. “Ultraviolet (UV) light” means electromagnetic radiation with a wavelength in a range from about 200 nm to about 450 nm.

According to the present application, the term “linearly polarised light” means light, which is at least partially linearly polarized. Preferably, the aligning light is linearly polarized with a degree of polarization of more than 5:1. Wavelengths, intensity and energy of the linearly polarised light are chosen depending on the photosensitivity of the photoalignable material. Typically, the wavelengths are in the UV-A, UV-B and/or UV-C range or in the visible range. Preferably, the linearly polarised light comprises light of wavelengths less than 450 nm, more preferably less than 420 nm at the same time the linearly polarised light preferably comprises light of wavelengths longer than 280 nm, preferably more than 320 nm, more preferably over 350 nm.

The Irradiance (Eₑ) or radiation power is defined as the power of electromagnetic radiation (dθ per unit area (dA) incident on a surface:

$${E}_{e} = d θ / dA .$$

The radiant exposure or radiation dose (Hₑ), is as the irradiance or radiation power (Eₑ) per time (t):

$${H}_{e} = {E}_{e} \cdot t .$$

On the molecular level, the birefringence of a liquid crystal depends on the anisotropy of the polarizability (Δα=α_{∥}−α_{⊥}). “Polarisability” means the ease with which the electron distribution in the atom or molecule can be distorted. The polarizability increases with greater number of electrons and a more diffuse electron cloud. The polarizability can be calculated using a method described in e.g. Jap. J. Appl. Phys. 42, (2003) p. 3463.

The “optical retardation” at a given wavelength R(λ) (in nm) of a layer of liquid crystalline or birefringent material is defined as the product of birefringence at that wavelength Δn(λ) and layer thickness d (in nm) according to the following equation:

$$R ( λ ) = Δ n ( λ ) \cdot d$$

The optical retardation R represents the difference in the optical path lengths in nanometres travelled by S-polarised and P-polarised light whilst passing through the birefringent material. “On-axis” retardation means the retardation at normal incidence to the sample surface.

The retardation (R(λ)) of a material can be measured using a spectroscopic ellipsometer, for example the M2000 spectroscopic ellipsometer manufactured by J. A. Woollam Co. This instrument can measure the optical retardance in nanometres of a birefringent sample e.g., Quartz over a range of wavelengths typically, 370 nm to 2000 nm. From this data it is possible to calculate the dispersion (R(450)/R(550) or Δn(450)/Δn(550)) of a material.

A method for carrying out these measurements was presented at the National Physics Laboratory (London, UK) by N. Singh in October 2006 and entitled “Spectroscopic Ellipsometry, Part1—Theory and Fundamentals, Part 2—Practical Examples and Part 3—measurements”. In accordance with the measurement procedures described Retardation Measurement (RetMeas) Manual (2002) and Guide to WVASE (2002) (Woollam Variable Angle Spectroscopic Ellipsometer) published by J. A. Woollam Co. Inc (Lincoln, NE, USA). Unless stated otherwise, this method is used to determine the retardation of the materials, films and devices described in this invention.

The term “director” is known in prior art and means the preferred orientation direction of the long molecular axes (in case of calamitic compounds) or short molecular axes (in case of discotic compounds) of the liquid-crystalline or RM molecules. In case of uniaxial ordering of such anisotropic molecules, the director is the axis of anisotropy.

The term “alignment” or “orientation” relates to alignment (orientational ordering) of anisotropic units of material such as small molecules or fragments of big molecules in a common direction named “alignment direction”. In an aligned layer of liquid-crystalline or RM material the liquid-crystalline director coincides with the alignment direction so that the alignment direction corresponds to the direction of the anisotropy axis of the material.

The terms “uniform orientation” or “uniform alignment” of an liquid-crystalline or RM material, for example in a layer of the material, mean that the long molecular axes (in case of calamitic compounds) or the short molecular axes (in case of discotic compounds) of the liquid-crystalline or RM molecules are oriented substantially in the same direction. In other words, the lines of liquid-crystalline director are parallel.

The terms “homeotropic structure/alignment/orientation” refer to a film wherein the optical axis is substantially perpendicular to the film plane.

The terms “planar structure/alignment/orientation” refer to a film wherein the optical axis is substantially parallel to the film plane.

All temperatures, such as, for example, the melting point T(C,N) or T(C,S), the transition from the smectic (S) to the nematic (N) phase T(S,N) and the clearing point T(N,I) of the liquid crystals, are quoted in degrees Celsius. All temperature differences are quoted in differential degrees.

In case of doubt the definitions as given in C. Tschierske, G. Pelzl and S. Diele, Angew. Chem. 2004, 116, 6340-6368 shall apply.

If in the formulae shown above and below a group R, including any variations thereof such as R¹, R⁰, R⁰⁰, R⁰*, R¹¹, R*, R**, R^{C}, R³, R⁴ etc., or L denotes an alkyl radical and/or an alkoxy radical, this may be straight-chain or branched. It is preferably straight-chain, has 2, 3, 4, 5, 6 or 7 C atoms and accordingly preferably denotes ethyl, propyl, butyl, pentyl, hexyl, heptyl, ethoxy, propoxy, butoxy, pentoxy, hexyloxy or heptyloxy, furthermore methyl, octyl, nonyl, decyl, undecyl, dodecyl, tridecyl, tetradecyl, pentadecyl, methoxy, octyloxy, nonyloxy, decyloxy, undecyloxy, dodecyloxy, tridecyloxy or tetradecyloxy.

If in the formulae shown above and below a group R including any variations thereof such as R¹, R⁰, R⁰⁰, R*⁰, R¹¹, R²², R^{C}, R³, R⁴ etc., or L denotes an alkyl radical and/or an alkoxy radical, this may be straight-chain or branched. It is preferably straight-chain, has 2, 3, 4, 5, 6 or 7 C atoms and accordingly preferably denotes ethyl, propyl, butyl, pentyl, hexyl, heptyl, ethoxy, propoxy, butoxy, pentoxy, hexyloxy or heptyloxy, furthermore methyl, octyl, nonyl, decyl, undecyl, dodecyl, tridecyl, tetradecyl, pentadecyl, methoxy, octyloxy, nonyloxy, decyloxy, undecyloxy, dodecyloxy, tridecyloxy or tetradecyloxy.

If in the formulae shown above and below a group R including any variations thereof such as R¹, R⁰, R⁰⁰, R⁰*, R¹¹, R²², R^{C}, R³, R⁴etc., or L denotes an alkyl radical wherein one or more CH₂ groups are replaced by S, this may be straight-chain or branched. It is preferably straight-chain, has 1, 2, 3, 4, 5, 6 or 7 C atoms and accordingly preferably denotes thiomethyl, thioethyl, thiopropyl, thiobutyl, thiopentyl, thiohexyl or thioheptyl.

Oxaalkyl preferably denotes straight-chain 2-oxapropyl (=methoxymethyl), 2-oxabutyl (=ethoxymethyl) or 3-oxabutyl (=2-methoxyethyl), 2-, 3- or 4-oxapentyl, 2-, 3-, 4- or 5-oxahexyl, 2-, 3-, 4-, 5- or 6-oxaheptyl, 2-, 3-, 4-, 5-, 6- or 7-oxaoctyl, 2-, 3-, 4-, 5-, 6-, 7- or 8-oxanonyl, 2-, 3-, 4-, 5-, 6-, 7-, 8- or 9-oxadecyl.

If in the formulae shown above and below a group R including any variations thereof such as R¹, R⁰⁰, R⁰⁰, R*⁰, R¹¹, R²², R^{C}, R³, R⁴ etc., or L denotes an alkoxy or oxaalkyl group it may also contain one or more additional oxygen atoms, provided that oxygen atoms are not linked directly to one another.

In another preferred embodiment, one or more of R including any variations thereof such as R¹, R⁰, R⁰⁰, R*⁰, R¹¹, R²², R^{C}, R³, R⁴ etc., or L are selected from the group consisting of

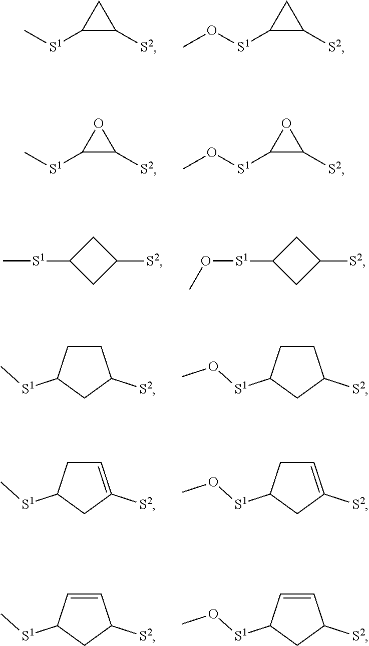

—S¹—F, —O—S¹—F, —O—S¹—O—S₂, wherein S¹ is C₁₋₁₂-alkylene or C₂₋₁₂-alkenylene and S² is H, C₁₋₁₂-alkyl or C₂₋₁₂-alkenyl, and very preferably are selected from the group consisting of

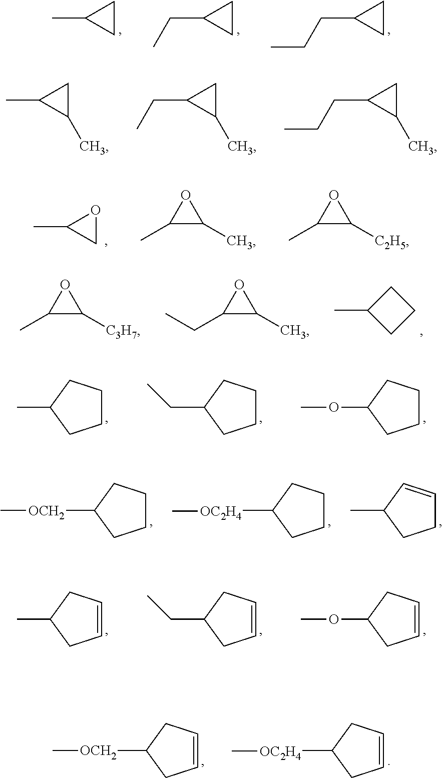

—OCH₂OCH₃, —O(CH₂)₂OCH₃, —O(CH₂)₃OCH₃, —O(CH₂)₄OCH₃, —O(CH₂)₂F, —O(CH₂)₃F and —O(CH₂)₄F.

If in the formulae shown above and below a group R including any variations thereof such as R¹, R⁰, R⁰⁰, R*⁰, R¹¹, R²², R^{C}, R³, R⁴ etc., or L denotes an alkyl radical in which one CH₂ group has been replaced by —CH═CH—, this may be straight-chain or branched. It is preferably straight-chain and has 2 to 10 C atoms. Accordingly, it denotes, in particular, vinyl, prop-1- or -2-enyl, but-1-, -2- or -3-enyl, pent-1-, -2-, -3- or -4-enyl, hex-1-, -2-, -3-, -4- or -5-enyl, hept-1-, -2-, -3-, -4-, -5- or -6-enyl, oct-1-, -2-, -3-, -4-, -5-, -6- or -7-enyl, non-1-, -2-, -3-, -4-, -5-, -6-, -7- or -8-enyl, dec-1-, -2-, -3-, -4-, -5-, -6-, -7-, -8- or -9-enyl.

If in the formulae shown above and below a group R including any variations thereof such as R¹, R⁰, R⁰⁰, R*⁰, R¹¹, R²², R^{C}, R³, R⁴ etc., or L denotes an alkyl or alkenyl radical which is at least monosubstituted by halogen, this radical is preferably straight-chain, and halogen is preferably F or Cl. In the case of polysubstitution, halogen is preferably F. The resultant radicals also include perfluorinated radicals. In the case of monosubstitution, the fluorine or chlorine substituent may be in any desired position, but is preferably in the ω-position.

Above and below,

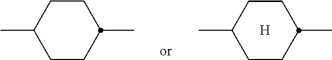

denotes a trans-1,4-cyclohexylene ring, and

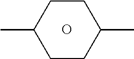

denotes a 1,4-phenylene ring.

Halogen is preferably F or Cl, very preferably F.

The group —CR⁰═CR⁰⁰— is preferably —CH═CH—.

—OC—, —CO—, —C(═O)— and —C(O)— denote a carbonyl group, i.e.

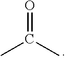

Preferred substituents L, are, for example, F, Cl, Br, I, —CN, —NO₂, —NCO, —NCS, —OCN, —SCN, —C(═O)N(R^{x})₂, —C(═O)Y¹, —C(═O)R^{x}, —N(R^{x})₂, straight-chain or branched alkyl, alkoxy, alkylcarbonyl, alkoxycarbonyl, alkylcarbonyloxy or alkoxycarbonyloxy each having 1 to 25 C atoms, in which one or more H atoms may optionally be replaced by F or Cl, optionally substituted silyl having 1 to 20 Si atoms, or optionally substituted aryl having 6 to 25, preferably 6 to 15, C atoms,
wherein R^{x} denotes H, F, Cl, CN, or straight chain, branched or cyclic alkyl having 1 to 25 C atoms, wherein one or more non-adjacent CH₂-groups are optionally replaced by —O—, —S—, —CO—, —CO—O—, —O—CO—, —O—CO—O— in such a manner that O- and/or S-atoms are not directly connected with each other, and wherein one or more H atoms are each optionally replaced by F, Cl, P—or P-Sp-, and Y¹ denotes halogen.

Particularly preferred substituents L are, for example, F, Cl, CN, NO₂, CH₃, C₂H₅, OCH₃, SCH₃, OC₂H₅, SC₂H₅, COCH₃, COC₂H₅, COOCH₃, COOC₂H₅, CF₃, OCF₃, OCHF₂, OC₂F₅, furthermore phenyl.

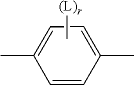

is preferably

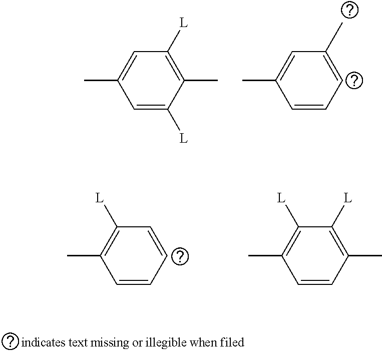

in which L has one of the meanings indicated above.

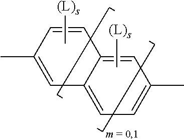

means

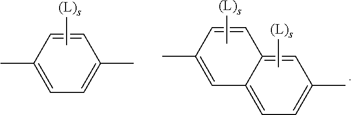

Throughout the application, the term “aryl and heteroaryl groups” encompass groups, which can be monocyclic or polycyclic, i.e. they can have one ring (such as, for example, phenyl) or two or more rings, which may also be fused (such as, for example, naphthyl) or covalently linked (such as, for example, biphenyl), or contain a combination of fused and linked rings.

Heteroaryl groups contain one or more heteroatoms, preferably selected from O, N, S and Se. Particular preference is given to mono-, bi- or tricyclic aryl groups having 6 to 25 C atoms and mono-, bi- or tricyclic heteroaryl groups having 2 to 25 C atoms, which optionally contain fused rings, and which are optionally substituted. Preference is furthermore given to 5-, 6- or 7-membered aryl and heteroaryl groups, in which, in addition, one or more CH groups may be replaced by N, S or O in such a way that 0 atoms and/or S atoms are not linked directly to one another. Preferred aryl groups are, for example, phenyl, biphenyl, terphenyl, [1,1′:3′,1″]¬¬terphenyl-2′-yl, naphthyl, anthracene, binaphthyl, phenanthrene, pyrene, dihydropyrene, chrysene, perylene, tetracene, pentacene, benzopyrene, fluorene, indene, indenofluorene, spirobifluorene, more preferably 1,4-phenylene, 4,4′-biphenylene, 1,4-tephenylene.

Preferred heteroaryl groups are, for example, 5 membered rings, such as pyrrole, pyrazole, imidazole, 1,2,3-triazole, 1,2,4-triazole, tetrazole, furan, thiophene, selenophene, oxazole, isoxazole, 1,2 thiazole, 1,3-thiazole, 1,2,3-oxadiazole, 1,2,4 oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 6 membered rings, such as pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, or condensed groups, such as indole, iso-indole, indolizine, indazole, benzimidazole, benzotriazole, purine, naphthimidazole, phenanthrimidazole, pyridimidazole, pyrazinimidazole, quinoxalinimidazole, benzoxazole, naphthoxazole, anthroxazole, phen-anthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]-thiophene, dithienothiophene, isobenzothiophene, dibenzothiophene, benzothiadiazothiophene, or combinations of these groups. The heteroaryl groups may also be substituted by alkyl, alkoxy, thioalkyl, fluorine, fluoroalkyl or further aryl or heteroaryl groups.

In a group

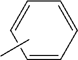

the single bond shown between the two ring atoms can be attached to any free position of the benzene ring.

—OC—, —CO—, —C(═O)— and —C(O)— denote a carbonyl group, i.e.

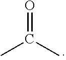

The polymerisable group P, including any variations thereof such as P⁰, P¹, P², P*⁰, is a group which is suitable for a polymerisation reaction, such as, for example, free-radical or ionic chain polymerisation, polyaddition or polycondensation, or for a polymer-analogous reaction, for example addition or condensation onto a main polymer chain. Particular preference is given to groups for chain polymerisation, in particular those containing a C═C double bond or —C≡C— triple bond, and groups which are suitable for polymerisation with ring opening, such as, for example, oxetane or epoxide groups.

Preferred groups P, including any variations thereof such as P⁰, P¹, P², P*⁰, are selected from the group consisting of CH₂═CW¹—CO—O—, CH₂═CW¹—CO—,

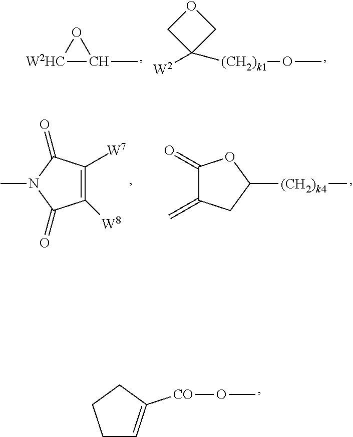

CH₂═CW²—(O)ₖ₃—, CW¹═CH—CO—(O)ₖ₃—, CW¹═CH—CO—NH—, CH₂═CW¹—CO—NH—, CH₃—CH═CH—O—, (CH₂═CH)₂CH—OCO—, (CH₂═CH—CH₂)₂CH—OCO—, (CH₂═CH)₂CH—O—, (CH₂═CH—CH₂)₂N—, (CH₂═CH—CH₂)₂N—CO—, HO—CW²W³—, HS—CW²W³—, HW²N—, HO—CW²W³—NH—, CH₂═CW¹—CO—NH—, CH₂═CH—(COO)ₖ₁-Phe-(O)ₖ₂—, CH₂═CH—(CO)ₖ₁-Phe-(O)ₖ₂—, Phe-CH═CH—, HOOC—, OCN— and W⁴W⁵W⁶Si—, in which W¹ denotes H, F, Cl, CN, CF₃, phenyl or alkyl having 1 to 5 C atoms, in particular H, F, Cl or CH₃, W² and W³ each, independently of one another, denote H or alkyl having 1 to 5 C atoms, in particular H, methyl, ethyl or n-propyl, W⁴, W⁵ and W⁶ each, independently of one another, denote Cl, oxaalkyl or oxacarbonylalkyl having 1 to 5 C atoms, W⁷ and W⁸ each, independently of one another, denote H, Cl or alkyl having 1 to 5 C atoms, Phe denotes 1,4-phenylene, which is optionally substituted by one or more radicals L as defined above which are other than P-Sp-, k₁, k₂ and k₃ each, independently of one another, denote 0 or 1, k₃ preferably denotes 1, and k₄ denotes an integer from 1 to 10.

Very preferred groups P, including any variations thereof such as P°, P¹, P², P*O, are selected from the group consisting of
CH₂═CW¹—CO—O—, CH₂═CW¹—CO—,

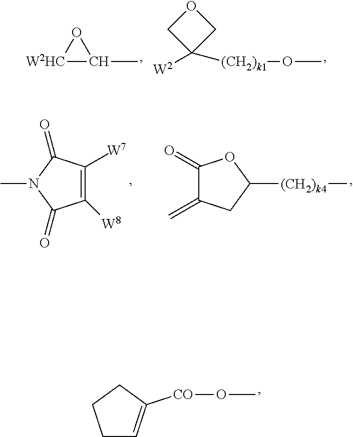

CH₂═CW²—O—, CH₂═CW²—
CW¹═CH—CO—(O)ₖ₃—, CW¹═CH—CO—NH—, CH₂═CW¹—CO—NH—,
(CH₂═CH)₂CH—OCO—, (CH₂═CH—CH₂)₂CH—OCO—, (CH₂═CH)₂CH—O—,
(CH₂═CH—CH₂)₂N—, (CH₂═CH—CH₂)₂N—CO—, CH₂═CW¹—CO—NH—,
CH₂═CH—(COO)ₖ₁-Phe-(O)ₖ₂—, CH₂═CH—(CO)ₖ₁-Phe-(O)ₖ₂—, Phe-CH═CH— and W⁴W⁵W⁶Si—, in which W¹ denotes H, F, Cl, CN, CF₃, phenyl or alkyl having 1 to 5 C atoms, in particular H, F, Cl or CH₃, W² and W³ each, independently of one another, denote H or alkyl having 1 to 5 C atoms, in particular H, methyl, ethyl or n-propyl, W⁴, W⁵ and W⁶ each, independently of one another, denote Cl, oxaalkyl or oxacarbonylalkyl having 1 to 5 C atoms, W⁷ and W⁸ each, independently of one another, denote H, Cl or alkyl having 1 to 5 C atoms, Phe denotes 1,4-phenylene, k₁, k₂ and k₃ each, independently of one another, denote 0 or 1, k₃ preferably denotes 1, and k₄ denotes an integer from 1 to 10.

Very particularly preferred groups P, including any variations thereof such as P⁰, P¹, P², P⁰, are selected from the group consisting of CH₂═CW¹—CO—O—, in particular CH₂═CH—CO—O—, CH₂═C(CH₃)—CO—O— and CH₂═CF—CO—O—, furthermore CH₂═CH—O—, (CH₂═CH)₂CH—O—CO—,

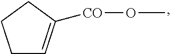

(CH₂═CH)₂CH—O—,

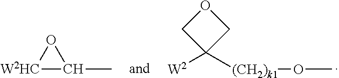

Further preferred polymerisable groups P, including any variations thereof such as P⁰, P¹, P², P*⁰, are selected from the group consisting of vinyloxy, acrylate, methacrylate, fluoroacrylate, chloroacrylate, oxetane and epoxide, most preferably from acrylate and methacrylate.

In another preferred embodiment of the invention, in a polymerisable compound as disclosed above and below, including compounds of formula I and its subformulae, all polymerisable groups have the same meaning, and preferably denote acrylate or methacrylate, very preferably acrylate.

The spacer group, including any variations thereof such as Sp⁰, Sp¹, Sp², Sp*⁰, when being different from a single bond, is preferably of the formula Sp″-X″, so that the respective radical P-Sp- etc. conforms to the formula P-Sp″—X″—, wherein
Sp″ denotes linear or branched alkylene having 1 to 20, preferably 1 to 12, C atoms, which is optionally mono- or polysubstituted by F, Cl, Br, I or CN and in which, in addition, one or more non-adjacent CH₂ groups may each be replaced, independently of one another, by —O—, —S—, —NH—, —N(R⁰)—, —Si(R⁰R⁰⁰)—, —CO—, —CO—O—, —O—CO—, —O—CO—O—, —S—CO—, —CO—S—, —N(R⁰⁰)—CO—O—, —O—CO—N(R⁰)—, —N(R⁰)—CO—N(R⁰⁰)—, —CH═CH— or —C≡C— in such a way that 0 and/or S atoms are not linked directly to one another,
X″ denotes —O—, —S—, —CO—, —CO—O—, —O—CO—, —O—CO—O—, —CO—N(R⁰)—, —N(R⁰)—CO—, —N(R⁰)—CO—N(R⁰⁰)—, —OCH₂—, —CH₂O—, —SCH₂—, —CH₂S—, —CF₂O—, —OCF₂—, —CF₂S—, —SCF₂—, —CF₂CH₂—, —CH₂CF₂—, —CF₂CF₂—, —CH═N—, —N═CH—, —N═N—, —CH═CR⁰—, —CY²═CY³—, —C≡C—, —CH═CH—CO—O—, —O—CO—CH═CH— or a single bond,
R⁰ and R⁰⁰ each, independently of one another, denote H or alkyl having 1 to 20 C atoms, and
Y² and Y³ each, independently of one another, denote H, F, Cl or CN.

X″ is preferably —O—, —S—, —CO—, —COO—, —OCO—, —O—COO—, —CO—NR⁰—, —NR⁰—CO—, —NR⁰—CO—NR⁰⁰— or a single bond.

Typical spacer groups Sp, including any variations thereof such as Sp⁰, Sp¹, Sp², Sp*⁰, and -Sp″—X″— are, for example, —(CH₂)ₚ₁—, —(CH₂)ₚ₁—O—, —(CH₂)ₚ₁—O—CO—, —(CH₂)ₚ₁—CO—O—, —(CH₂)ₚ₁—O—CO—O—, —(CH₂CH₂O)_{q1}—CH₂CH₂—, —CH₂CH₂—S—CH₂CH₂—, —CH₂CH₂—NH—CH₂CH₂— or —(SiR⁰R⁰⁰—O)ₚ₁—, in which p1 is an integer from 1 to 12, q1 is an integer from 1 to 3, and R⁰ and R⁰⁰ have the meanings indicated above.

Particularly preferred groups Sp, including any variations thereof such as Sp⁰, Sp¹, Sp², Sp*⁰, and -Sp″—X″— are —(CH₂)ₚ₁—, —(CH₂)ₚ₁—O—, —(CH₂)ₚ₁—O—CO—, —(CH₂)ₚ₁—CO—O—, —(CH₂)ₚ₁—O—CO—O—, in which p1 and q1 have the meanings indicated above.

Particularly preferred groups Sp″ are, in each case straight-chain, ethylene, propylene, butylene, pentylene, hexylene, heptylene, octylene, nonylene, decylene, undecylene, dodecylene, octadecylene, ethyleneoxyethylene, methyleneoxybutylene, ethylenethioethylene, ethylene-N-methyliminoethylene, 1-methylalkylene, ethenylene, propenylene and butenylene.

In another preferred embodiment of the invention, the polymerisable compounds as disclosed above and below, including compounds of formula I and its subformulae, contain a spacer group Sp, including any variations thereof such as Sp⁰, Sp¹, Sp², Sp*⁰, that is substituted by one or more polymerisable groups P, so that the group Sp-P etc. corresponds to Sp(P)ₛ, with s being ≥2 (branched polymerisable groups).

Preferred polymerisable compounds according to this preferred embodiment are those wherein s is 2, i.e., compounds which contain a group Sp(P)₂. Very preferred polymerisable compounds according to this preferred embodiment contain a group selected from the following formulae:

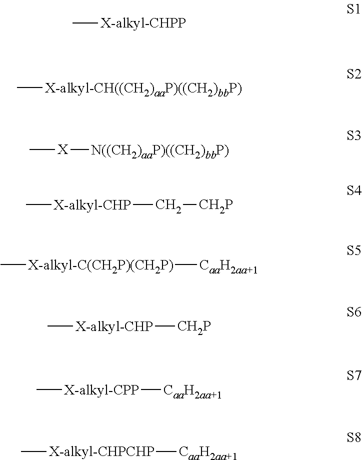

in which P is as defined in formula I,
alkyl denotes a single bond or straight-chain or branched alkylene having 1 to 12 C atoms which is unsubstituted or mono- or polysubstituted by F, Cl or CN and in which one or more non-adjacent CH₂ groups may each, independently of one another, be replaced by —C(R⁰)═C(R⁰)—, —C≡C—, —N(R⁰)—, —O—, —S—, —CO—, —CO—O—, —O—CO—, —O—CO—O— in such a way that 0 and/or S atoms are not linked directly to one another, where R⁰ has the meaning indicated above,
aa and bb each, independently of one another, denote 0, 1, 2, 3, 4, 5 or 6,
X has one of the meanings indicated for X″, and is preferably O, CO, SO₂, O—CO—, CO—O or a single bond.

Preferred groups Sp(P)₂ are selected from formulae S1, S2 and S3.

Very preferred groups Sp(P)₂ are selected from the following subformulae:

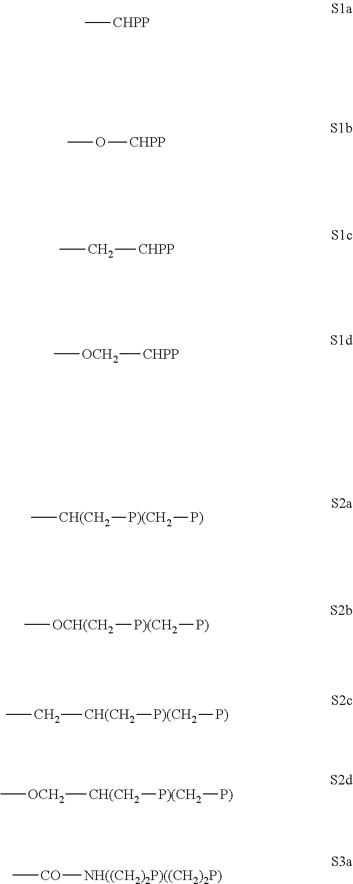

### DETAILED DESCRIPTION

The compounds of formula I are characterized I that they contain a spacer group which is linked via an acetylene group to a phenylene-1,4-diyl or naphthalene-2,6-diyl group which forms part of the mesogenic core that further contains two C—C triple bonds between aromatic rings like phenylene or naphthalene. It was surprisingly found that these compounds show an increase of the extraordinary refractive index nₑ. Moreover, their maximum absorbance peak is shifted towards lower wavelengths, with a steepened pitch of the UV absorbance curve, which results in less coloring of the RMs and RM mixtures of the present invention and the LCP films made thereof.

In the compounds of formula I and its subformulae as described above and below, P is preferably selected from the group consisting of vinyloxy, acrylate, methacrylate, fluoroacrylate, chloroacrylate, oxetane and epoxide, very preferably from acrylate and methacrylate, most preferably acrylate.

Further preferred are compounds of formula I and its subformulae as described above and below, wherein all polymerisable groups P that are present in the compound have the same meaning, and very preferably denote acrylate or methacrylate, most preferably acrylate.

Further preferred are compounds of formula I and its subformulae as described above and below, which contain one, two, three or four groups P-Sp, very preferably two or three groups P-Sp.

Further preferred are compounds of formula I and its subformulae as described above and below, wherein R¹¹ is P-Sp-.

Further preferred are compounds of formula I and its subformulae as described above and below, wherein R¹¹ is different from P-Sp- and is preferably selected from F, Cl, CN, CF₃, CCl₃, CH═CH₂, or alkoxy, thioalkyl, alkylcarbonyl, alkoxycarbonyl, alkylcarbonyloxy or alkoxycarbonyloxy with 1 to 3 C atoms which is optionally fluorinated, more preferably from F, Cl, CN, OCH₃, SCH₃, OCF₃, CF₃, CH═CH₂, CHO, COCH₃, COOCH₃, COOC₂H₅ and CCl₃, very preferably from OCH₃, OCF₃ or CHO, most preferably from OCH₃ or OCF₃.

Further preferred are compounds of formula I and its subformulae as described above and below, wherein Sp¹ denotes —(CH₂)ₛ₁—, wherein s1 is an integer from 1 to 12, more preferably 3, 4, 5 or 6.

Further preferred are compounds of formula I and its subformulae as described above and below, wherein Sp denotes a single bond or —(CH₂)ₚ₁—, —O—(CH₂)ₚ₁—, —O—CO—(CH₂)ₚ₁, or —CO—O—(CH₂)ₚ₁, wherein p1 is 2, 3, 4, 5 or 6, and, if Sp is —O—(CH₂)ₚ₁—, —O—CO—(CH₂)ₚ₁ or —CO—O—(CH₂)ₚ₁ the O-atom or CO-group, respectively, is linked to the benzene ring.

Further preferred are compounds of formula I and its subformulae as described above and below, wherein Sp is a single bond.

Further preferred are compounds of formula I and its subformulae as described above and below, wherein Sp is different from a single bond.

Further preferred are compounds of formula I and its subformulae as described above and below, wherein m is 1.

Further preferred are compounds of formula I and its subformulae as described above and below, wherein at least one of B and C denotes naphthalene-2,6-diyl or naphthalene-1,4-diyl, very preferably naphthalene-2,6-diyl, which is optionally substituted by one or more groups L or P-Sp-.

Further preferred are compounds of formula I and its subformulae as described above and below, wherein L is P-Sp-, F, Cl, —CN, or straight chain, branched or cyclic alkyl having 1 to 25 C atoms, wherein one or more non-adjacent CH₂-groups are optionally replaced by —O—, —S—, —CO—, —CO—O—, —O—CO—, —O—CO—O—, CR⁰═CR⁰⁰—, —C≡C—,

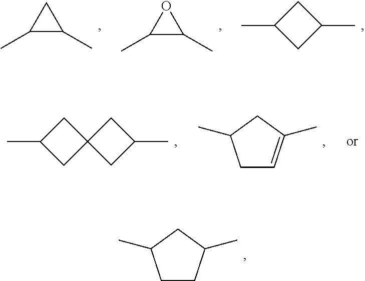

in such a manner that O- and/or S-atoms are not directly connected with each other, and wherein one or more H atoms are each optionally replaced by P-Sp-, F or Cl, or two substituents L that are connected to directly adjacent C atoms may also form a cycloalkyl or cycloalkenyl group with 5, 6, 7 or 8 C atoms.

Very preferred are compounds of formula I and its subformulae as described above and below, wherein L is F, Cl, CN or straight chain alkyl, alkoxy or thioalkyl having 1 to 6 C atoms or branched or cyclic alkyl, alkoxy or thioalkyl having 3 to 8 C atoms, most preferably F, Cl, CN, CH₃, OCH₃, SCH₃, C₂H₅, OC₂H₅ or SC₂H₅.

Further preferred are compounds of formula I and its subformulae as described above and below, wherein Z¹¹ and Z¹² denote —COO—, —OCO—, —C≡C— or a single bond, more preferably —C≡C— or a single bond, most preferably a single bond.

Preferably A and B in formula I are selected from the group consisting of

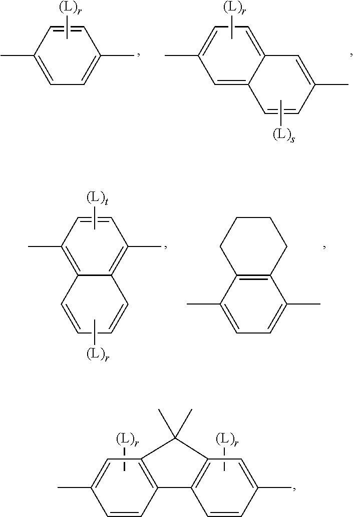

wherein at least one of A and B is selected from phenylene-1,4-diyl and napththalene-2,6-diyl, and wherein the individual radicals, independently of each other and on each occurrence identically or differently, have the following meanings
L P-Sp-, —CN, F, Cl, or alkyl, alkoxy or thioalkyl which is optionally fluorinated and has 1 to 6, preferably 1 to 3, more preferably 1 or 2 C atoms, preferably P-Sp-, —CN, F, Cl, OCH₃, SCH₃, C₂H₅, OC₂H₅, SC₂H₅, CHO, COCH₃, COOCH₃ or COOH,
r 0, 1, 2, 3 or 4, preferably 0, 1 or 2,
s 0, 1, 2 or 3, preferably 0 or 1,
t 0, 1 or 2, preferably 0 or 1.

More preferably rings A and B in formula I are selected from the group consisting of phenylene-1,4-diyl, naphthalene-1,4-diyl and naphthalene 2,6-diyl, all of which are optionally substituted by one or more groups L and/or P-Sp-, wherein not more than one of A and B may denote naphthalene-1,4-diyl.

Very preferably one, two, three, four or more of rings A and B in formula I are selected from the group consisting of

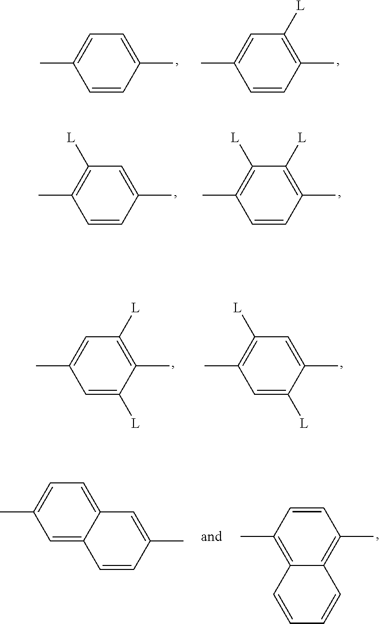

wherein not more than one of A and B may denote naphthalene-1,4-diyl and wherein L, on each occurrence identically or differently, denotes P-Sp-, —CN, F, Cl, or alkyl, alkoxy or thioalkyl which is optionally fluorinated and has 1 to 6, preferably 1 to 3, more preferably 1 or 2 C atoms, preferably P-Sp-, F, Cl, CN, CH₃, OCH₃, SCH₃, C₂H₅, OC₂H₅ or SC₂H₅.

Especially preferred are compounds of formula I, in particular wherein n=m=0, wherein the ring B is selected from the group consisting of phenylene-1,4-diyl, naphthalene-1,4-diyl and naphthalene-2,6-diyl, preferably phenylene-1,4-diyl, naphthalene-1,4-diyl and naphthalene-2,6-diyl, all of which are optionally mono- or disubstituted by L and/or P-Sp-.

Preferably ring C in formula I is selected from the group consisting of

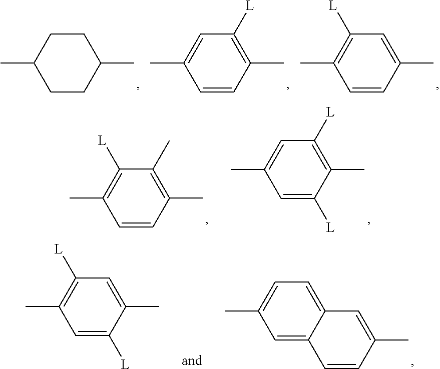

wherein L, on each occurrence identically or differently, denotes P-Sp-, —CN, F, Cl, or alkyl, alkoxy or thioalkyl which is optionally fluorinated and has 1 to 6, preferably 1 to 3, more preferably 1 or 2 C atoms, preferably P-Sp-, F, Cl, CN, CH₃, OCH₃, SCH₃, C₂H₅, OC₂H₅ or SC₂H₅.

Further preferred are compounds of formula I wherein m=1 and/or at least one of rings B and C denotes naphthalene-2,6-diyl.

Further preferred are compounds of formula I wherein m=0, preferably those wherein n=0, wherein rings B and C together with the phenylene-1,4-diyl group form a group selected from the following formulae or their mirror images, wherein * denotes the linkage to the acetylene group in formula I:

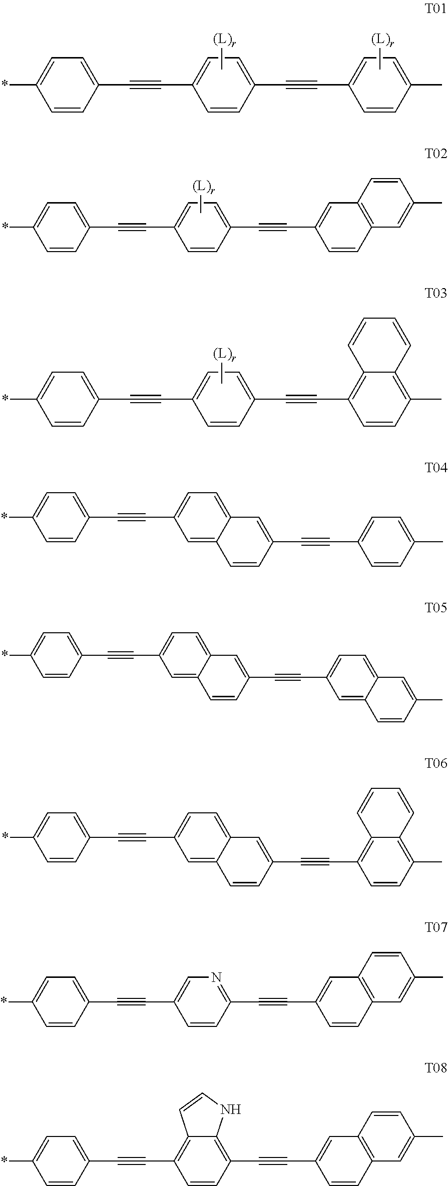

wherein the naphthalene groups are optionally substituted with one or two groups L, r is 0, 1, 2, 3 or 4, preferably 0, 1 or 2, and L is as defined in formula I.

Further preferred are compounds of formula I wherein m=1, preferably those wherein n=0, wherein rings B and C together with the naphthalene-2,6-diyl group form a group selected from the following formulae or their mirror images, wherein * denotes the linkage to the acetylene group in formula I:

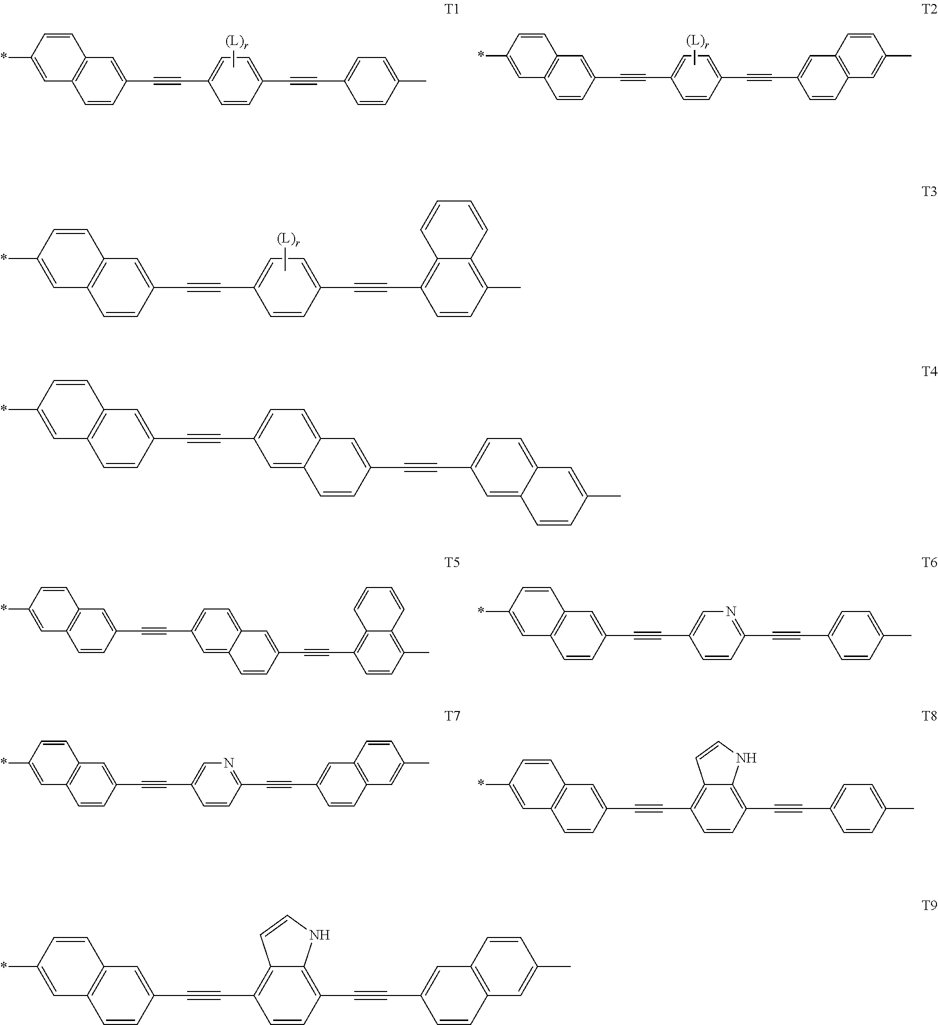

wherein the naphthalene groups are optionally substituted with one or two groups L, r is 0, 1, 2, 3 or 4, preferably 0, 1 or 2, and L is as defined in formula I.

In formulae T01 to T08 and T1 to T9, preferably L on each occurrence identically or differently denotes P-Sp-, —CN, F, Cl, or alkyl, alkoxy or thioalkyl which is optionally fluorinated and has 1 to 6, preferably 1 to 3, more preferably 1 or 2 C atoms, very preferably P-Sp-, F, Cl, CN, CH₃, OCH₃, SCH₃, C₂H₅, OC₂H₅ or SC₂H₅, most preferably CH₃ or C₂H₅, and r is preferably 0, 1, 2 or 3, very preferably 0, 1 or 2.

Especially preferred are the groups of formulae T01, T02, T03, T1, T2 and T3.

Very preferred compounds of formula I are selected from the following subformulae:

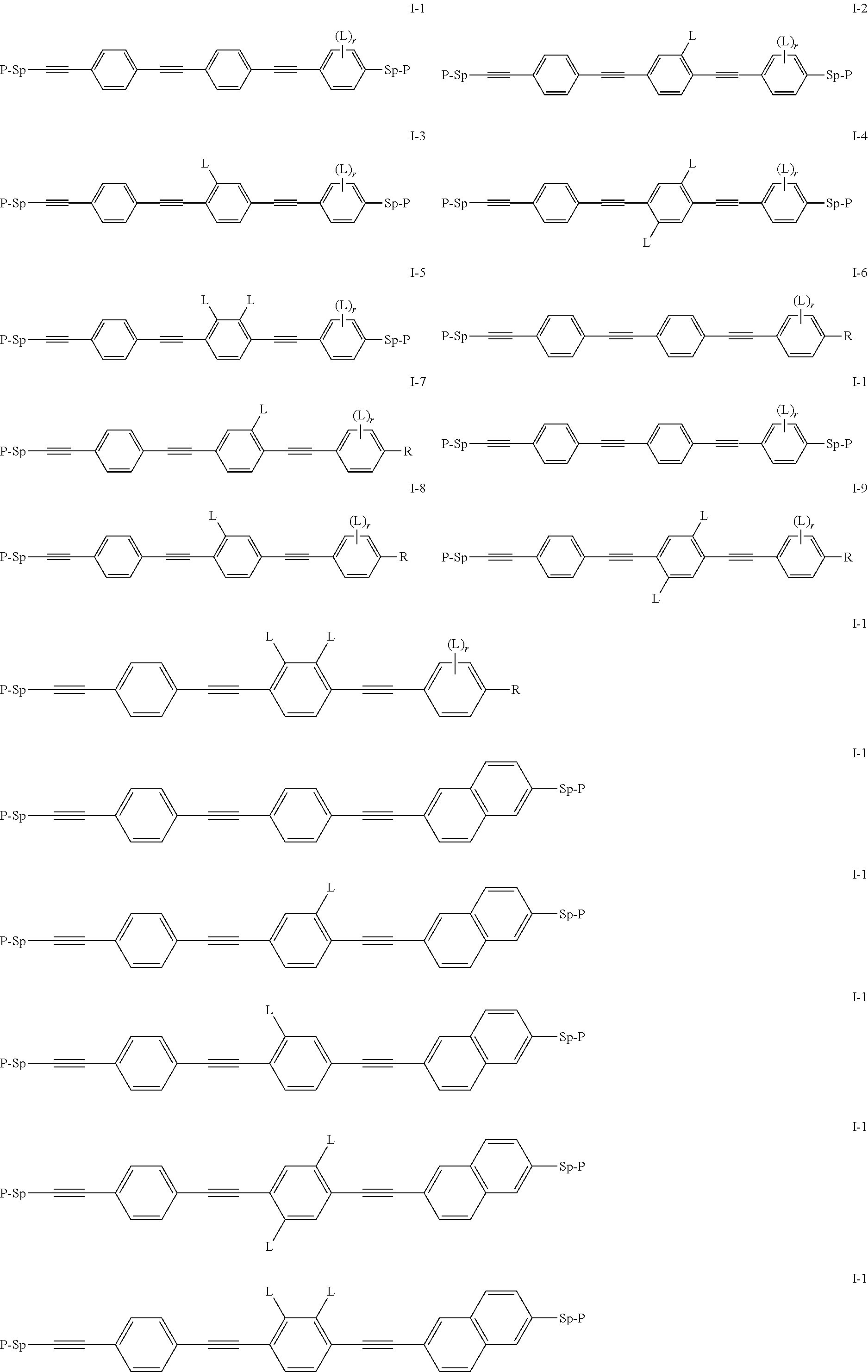

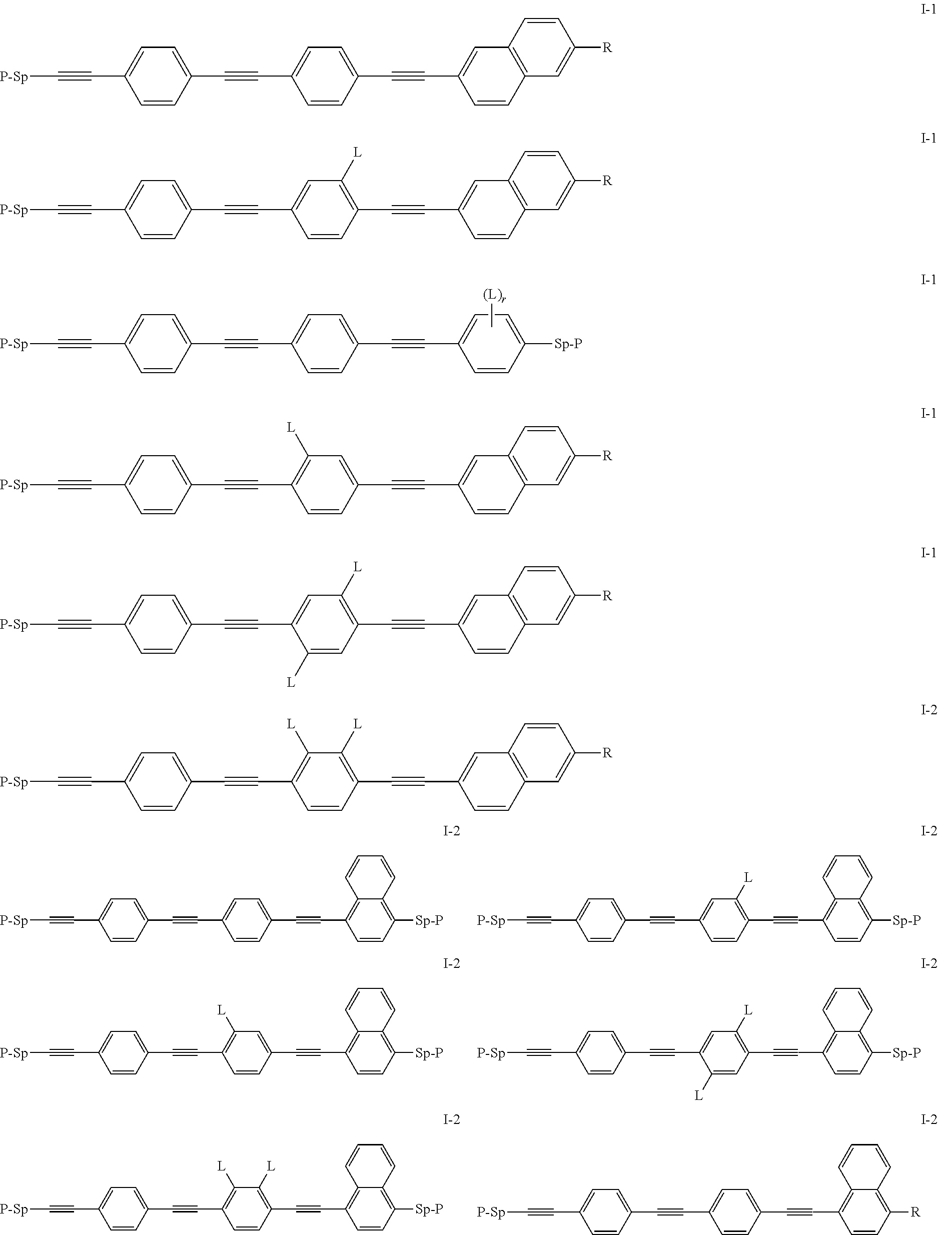

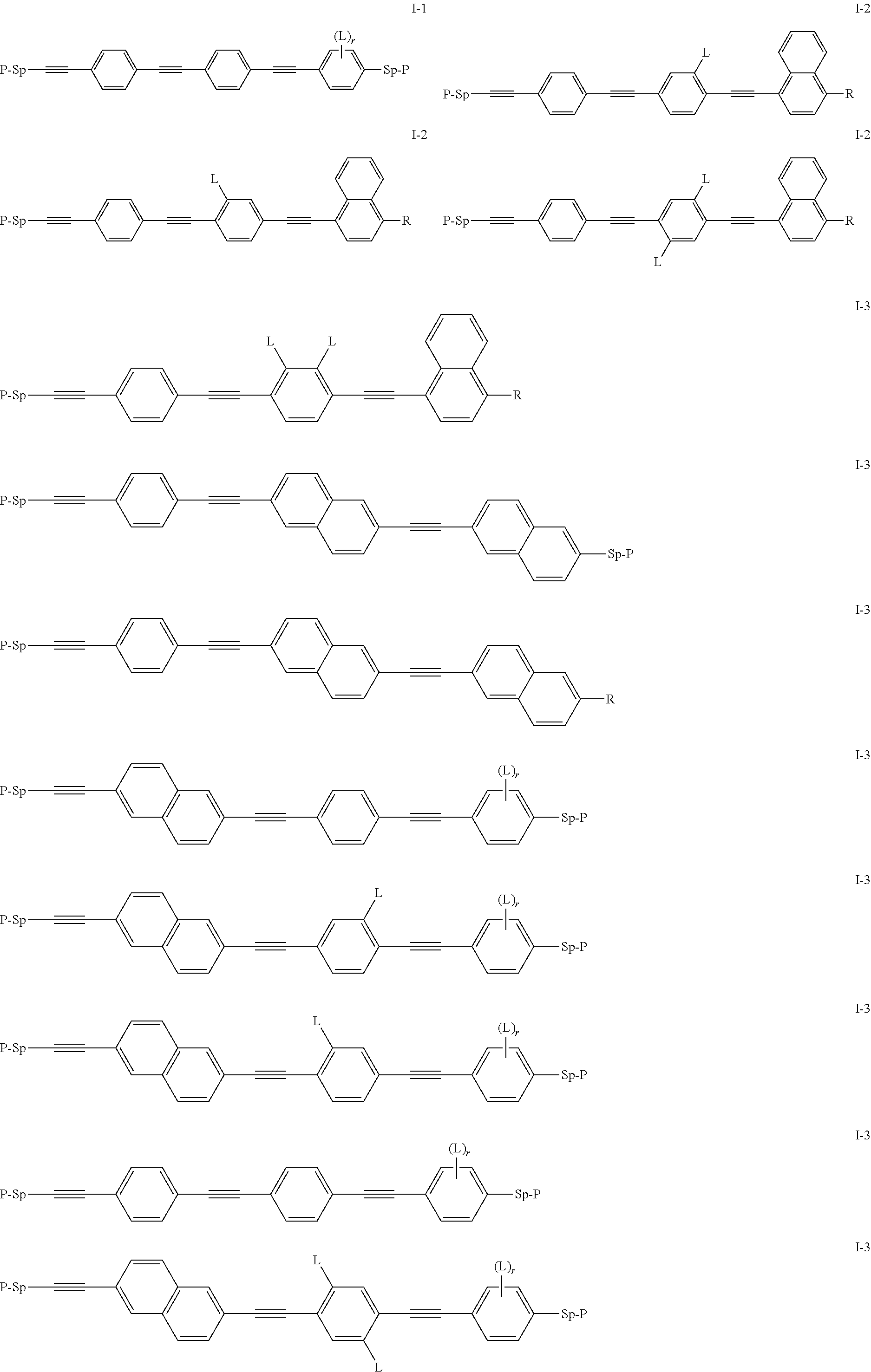

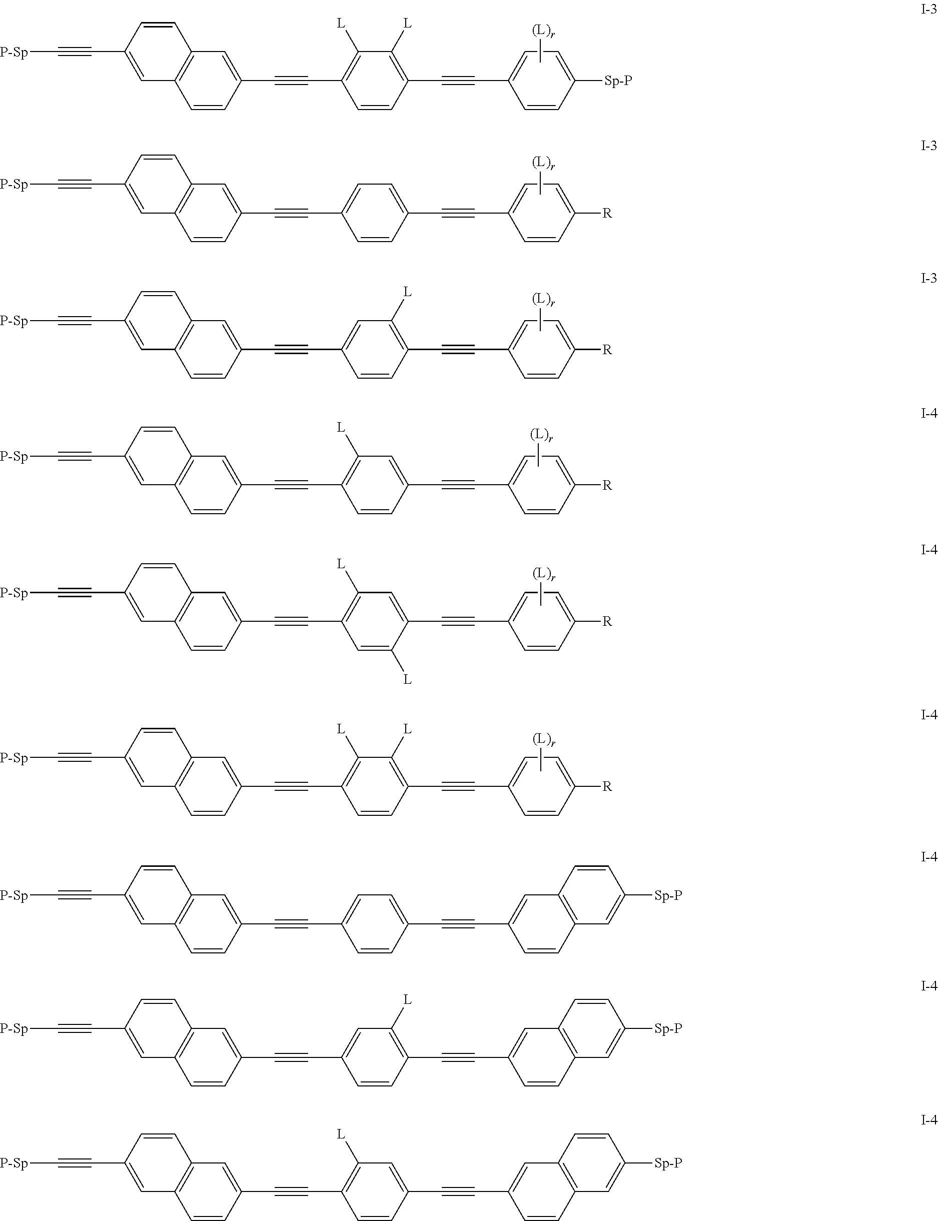

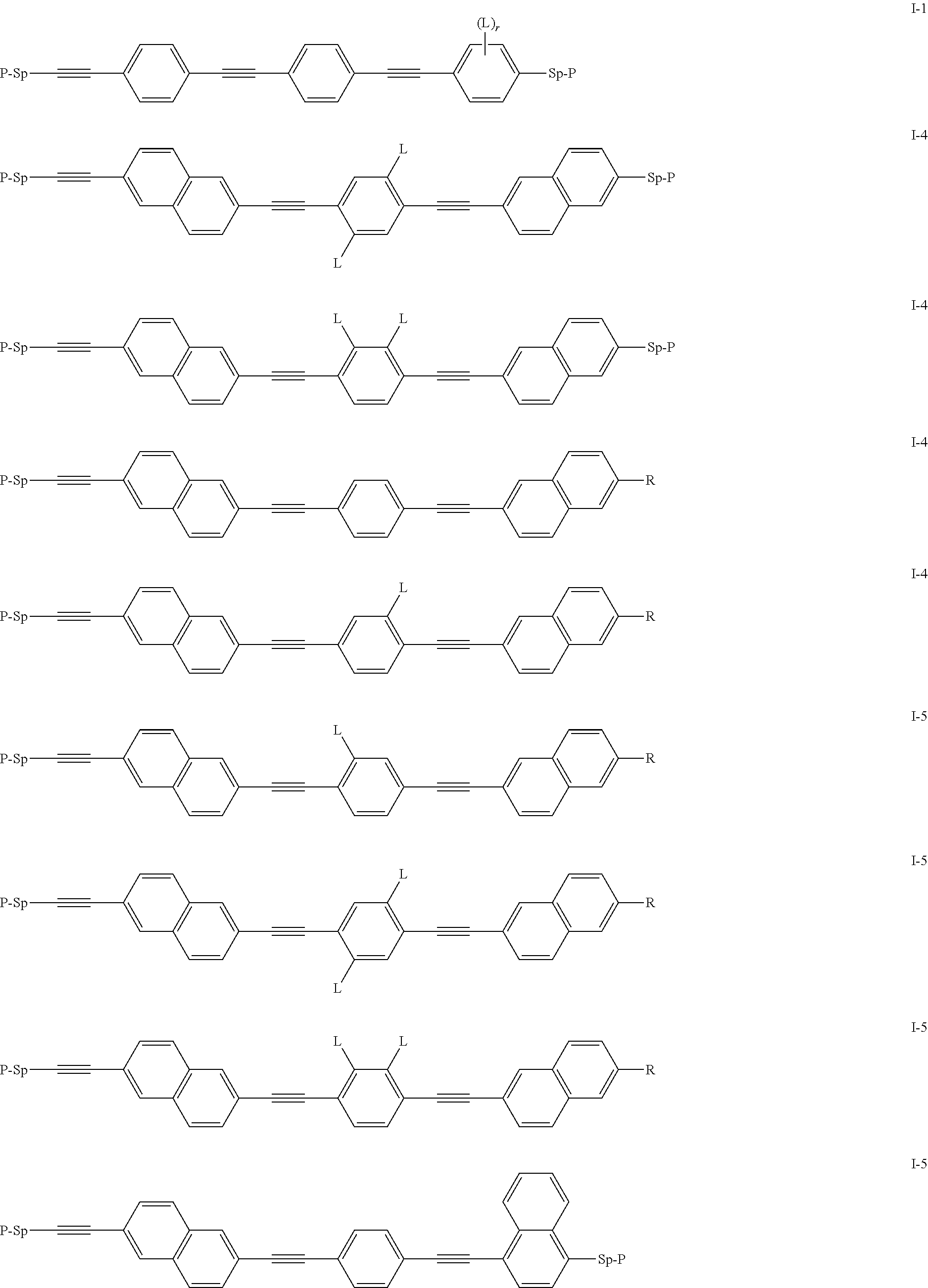

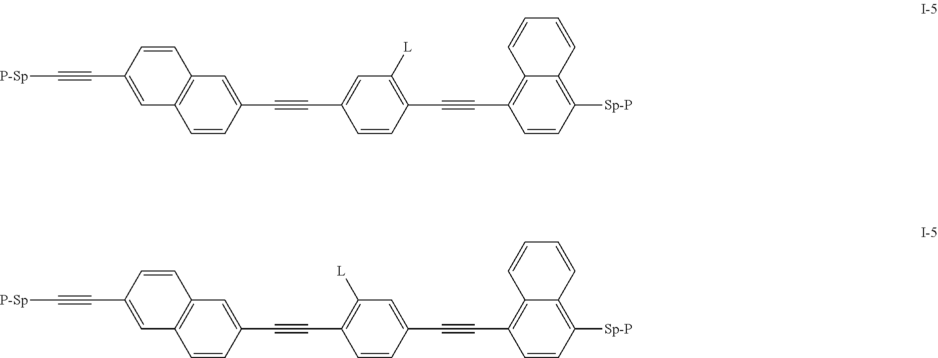

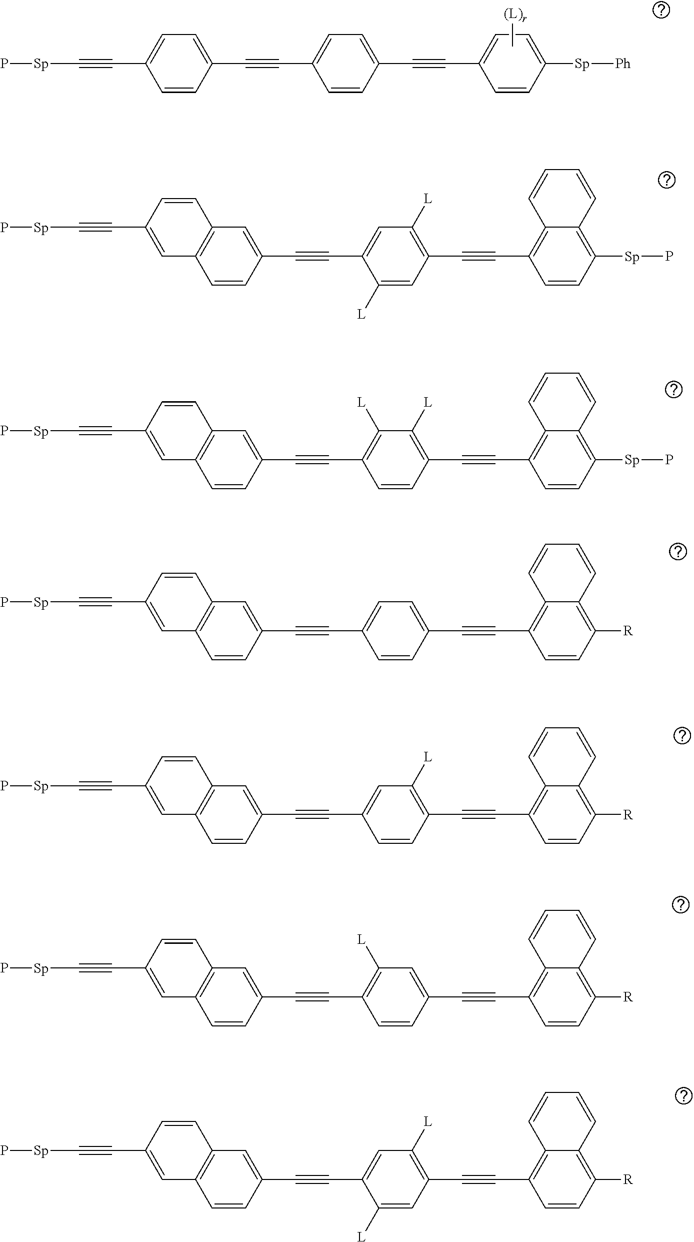

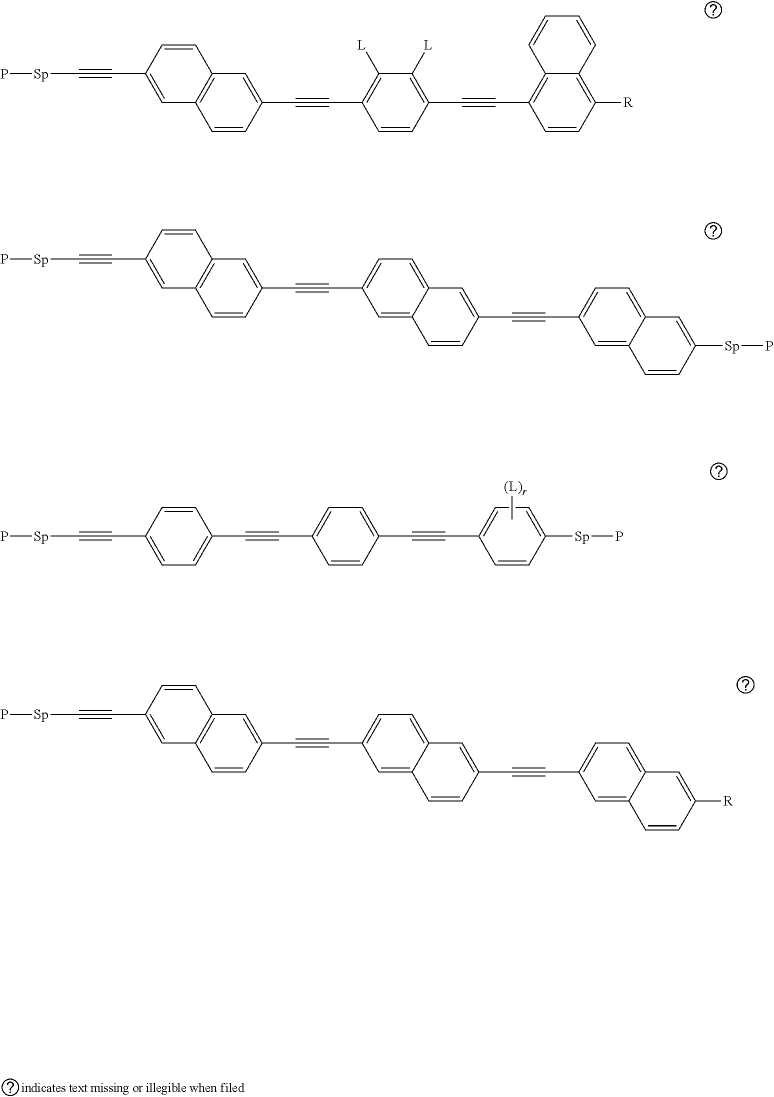

wherein the naphthalene groups are optionally substituted with one or two groups L, and P, Sp, L and r, independently of each other and on each occurrence identically or differently, have the meanings given above, and R has one of the meanings given for R¹¹ in formula I, and preferably denotes OCH₃ or SCH₃, very preferably OCH₃. L is preferably selected from F, Cl, CN, CH₃, OCH₃, SCH₃, C₂H₅, OC₂H₅ or SC₂H₅. P is preferably acrylate.

Further preferred are compounds of the formulae I and I-1 to I-64 wherein one of the two groups Sp is a single bond and the other group Sp is different from a single bond.

Further preferred compounds of the formulae I and I-1 to I-64 are selected from the following preferred embodiments including any combination thereof:
n=0, and/or
ring C denotes phenylene-1,4-diyl which is substituted by alkyl, alkoxy or thioalkyl with 1 to 3, preferably 1 or 2 C atoms, more preferably methyl or ethyl, most preferably ethyl, or
ring C denotes naphthalene-2,6-diyl, which is optionally substituted by one or more groups L or P-Sp-, and/or
ring B denotes naphthalene-2,6-diyl, which is optionally substituted by one or more groups L or P-Sp-, and/or
m=1 and/or at least one of B and C denotes naphthalene-2,6-diyl, which is optionally substituted by one or more groups L or P-Sp-,
m=0 and at least one of B and C denotes naphthalene-2,6-diyl, which is optionally substituted by one or more groups L or P-Sp-,
P denotes acrylate or methacrylate and/or
Sp¹ denotes —(CH₂)ₛ₁—, wherein s1 is an integer from 1 to 1, preferably 3, 4, 5 or 6, and/or
Sp denotes Sp″—X″, preferably, -Sp″—X″— denotes —(CH₂)ₚ₁—, —(CH₂)ₚ₁—O—, —(CH₂)ₚ₁—O—CO—, —(CH₂)ₚ₁—CO—O—, —(CH₂)ₚ₁—O—CO—O—, —(CH₂CH₂O)_{q1}—CH₂CH₂—, —CH₂CH₂—S—CH₂CH₂—, or —CH₂CH₂—NH—CH₂CH₂—, in which p1 is an integer from 1 to 12, q1 is an integer from 1 to 3, and/or
L is selected from P-Sp-, F, Cl, CN, CH₃, OCH₃, SCH₃, C₂H₅, OC₂H₅ or SC₂H₅, very preferably CH₃ or C₂H₅, and r denotes 1, and/or
ring C is substituted by one L which denotes P-Sp-, preferably acrylate, and/or
R¹¹ is P-Sp-, or
R¹¹ is F, Cl, CN, OCH₃ or SCH₃, preferably OCH₃ or SCH₃, very preferably OCH₃.

The synthesis of the compounds of formula I and its subformulae can be carried by methods known per se to the person skilled in the art from the literature or in analogy thereto, as described for example in WO 2022/233908 A1.

The compounds of formula I, either taken alone or in combination with other RMs in an RM mixture, exhibit in particular and preferably at the same time, a high birefringence, exhibit a good solubility in commonly known organic solvents used in mass production, show an improved alignment in the RM mixture, have favorable transition temperatures, and show high resistance against yellowing after being exposed to UV light.

Another object of the invention is an RM mixture comprising one or more compounds of formula I, and optionally further comprising one or more reactive mesogens, preferably selected from mono- and direactive mesogens, which are different from formula I, and optionally further comprising one or more chiral compounds which are optionally polymerisable and/or isomerisable.

Preferably the RM mixture contains one or more, preferably 1 to 5, very preferably 1, 2 or 3, compounds selected from formulae I, preferably selected from formulae I-1 to I-64.

The concentration of the compounds of formula I or its subformulae in the RM mixture is preferably from 40 to 99%, more preferably from 50 to 98%, very preferably from 60 to 98%, most preferably from 70 to 98%.

In a preferred embodiment, the concentration of di- or multireactive RMs of formula I in the RM mixture is from 30 to 100%, very preferably from 45 to 90%.

In another preferred embodiment, the concentration of monoreactive RMs of formula I in the RM mixture is from 0 to 50%, more preferably from 0 to 30%, very preferably from 1 to 30%.

In another preferred embodiment, the achiral reactive mesogens of the RM mixture are exclusively selected from formula I and its subformulae.

In another preferred embodiment the RM mixture contains one or more, preferably exactly one, chiral compounds, preferably selected from polymerisable chiral compounds, very preferably selected from mono- or direactive chiral polymerisable compounds.

Suitable polymerisable chiral compounds preferably comprise one or more ring elements, linked together by a direct bond or via a linking group and, where two of these ring elements optionally may be linked to each other, either directly or via a linking group, which may be identical to or different from the linking group mentioned. The ring elements are preferably selected from the group of four-, five-, six- or seven-, preferably of five- or six-, membered rings.

Preferred polymerisable chiral compounds are selected from the formulae CRM1, CRM2 and CRM3:

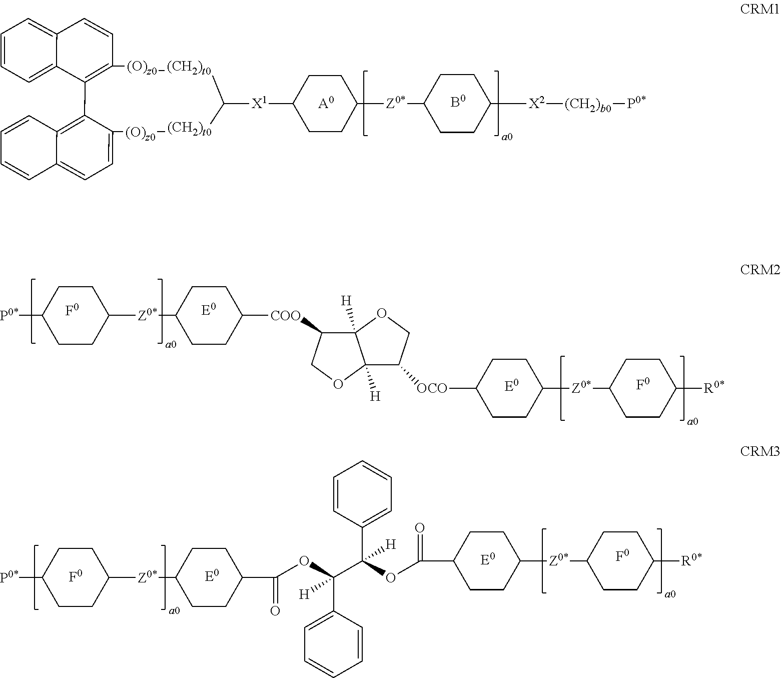

wherein the individual radicals, independently of each other and on each occurrence identically or differently, have the following meanings
P⁰* a polymerisable group,
Sp⁰* a spacer group or a single bond
R⁰* F, Cl, CN, alkyl, alkoxy, thioalkyl, alkylcarbonyl, alkoxycarbonyl, alkylcarbonyloxy or alkoxycarbonyloxy with 1 to 15, preferably 1 to 6 C atoms, P⁰*— or P⁰*-Sp*-,
A⁰, B⁰, E⁰, F⁰ 1,4-phenylene that is unsubstituted or substituted with 1, 2, 3 or 4 groups L, or trans-1,4-cyclohexylene,
L F, Cl, CN, P-Sp-, or alkyl, alkoxy, thioalkyl, alkylcarbonyl, alkoxycarbonyl, alkylcarbonyloxy or alkoxycarbonyloxy with 1 to 5 C atoms that is optionally fluorinated,
X¹, X²—O—, —COO—, —OCO—, —O—CO—O— or a single bond,
Z⁰* —COO—, —OCO—, —O—CO—O—, —OCH₂—, —CH₂O—, —CF₂O—, —OCF₂—, —CH₂CH₂—, —(CH₂)₄—, —CF₂CH₂—, —CH₂CF₂—, —CF₂CF₂—, —C≡C—, —CH═CH—, —CH═CH—COO—, —OCO—CH═CH— or a single bond, preferably —COO—, —OCO— or a single bond,
a0 0, 1 or 2, preferably 0 or 1,
b0 0 or an integer from 1 to 12, preferably 1 to 6,
t0 0, 1, 2 or 3,
z0 0 or 1, preferably 1,
   and wherein the naphthalene rings can additionally be substituted with one or more identical or different groups L.

Further preferred are the stereoisomers of formula CRM2 wherein the central isosorbide unit is replaced by an isomannide or isoidide unit.

The compounds of formula CRM1 are preferably selected from the following formula:

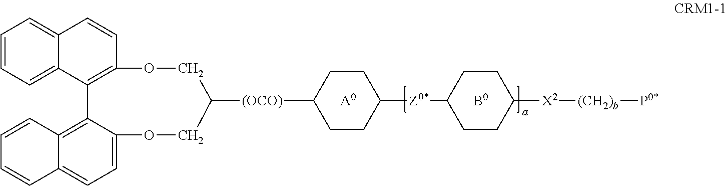

wherein A⁰, B⁰, Z⁰*, X², P⁰*, a and b have the meanings given in formula CRMa or one of the preferred meanings given above and below, and (OCO) denotes —O—CO— or a single bond.

Especially preferred compounds of formula CRM are selected from the group consisting of the following subformulae:

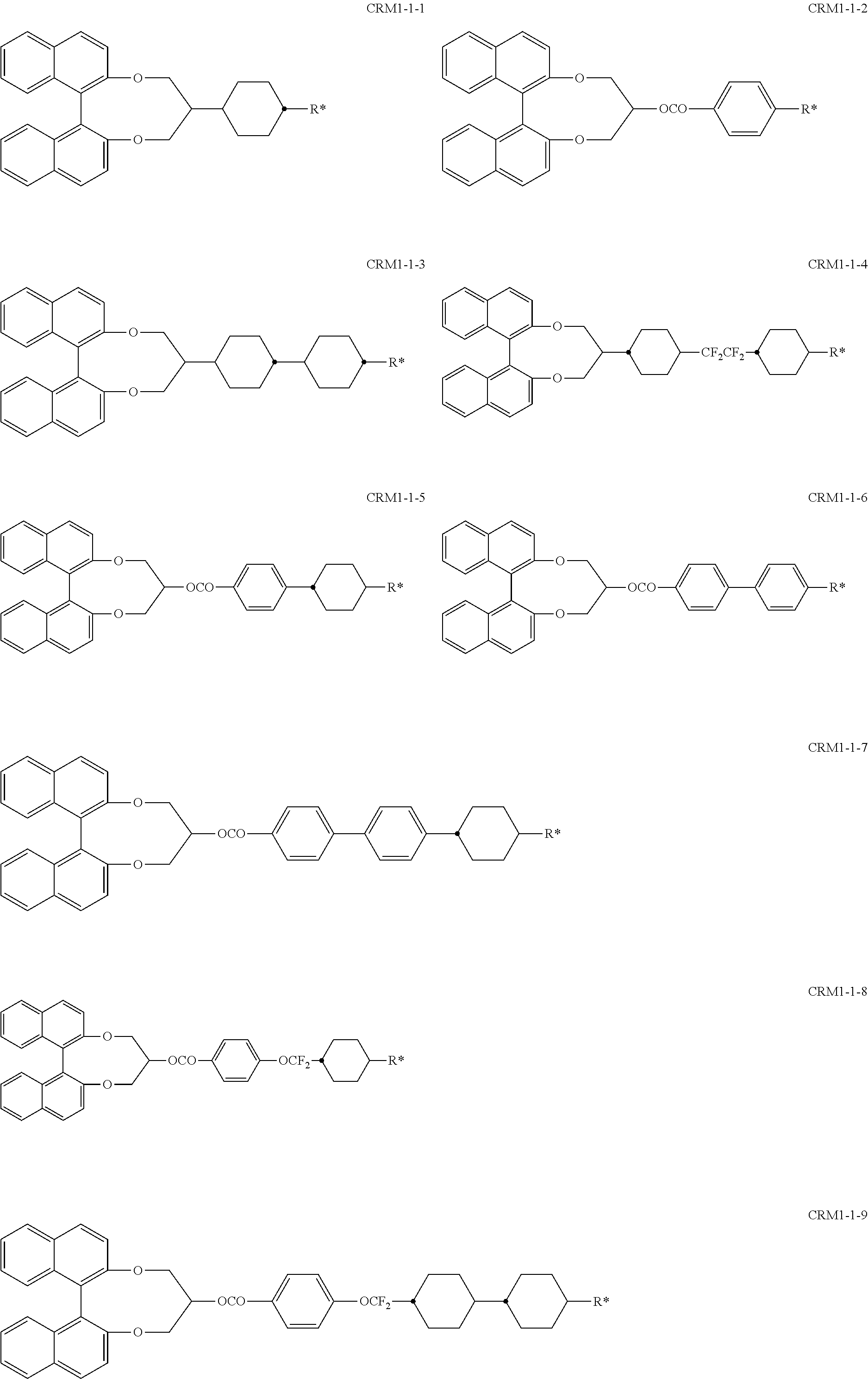

wherein R* is —X²—(CH₂)ₜ—P⁰* as defined in formula CRM1-1, and the benzene and naphthalene rings are unsubstituted or substituted with 1, 2, 3 or 4 groups L as defined above and below.

In case one or more polymerisable chiral compounds are present, their concentration in the RM mixture is preferably from 0.1 to 10%, more preferably from 0.5 to 8% by weight of the total RM mixture.

Preferably the polymerisable chiral compounds have alone or in combination with each other an absolute value of the helical twisting power (|HTPₜₒₜₐₗ|) of 20 μm⁻¹ or more, preferably of 40 μm⁻¹ or more, more preferably in the range of 60 μm⁻¹ or more, most preferably in the range of 80 μm⁻¹ or more to 260 μm⁻¹.

In another preferred embodiment the RM mixture according to the present invention does not contain any chiral compounds.

The RM mixture preferably exhibits a nematic phase or, in case a chiral compound is present, a chiral nematic (also referred to as “cholesteric”) LC phase, or a chiral smectic LC phase and a chiral nematic LC phase, very preferably a nematic or chiral nematic LC phase at room temperature.

The RM mixture preferably has a birefringence (□n) in the range from 0.18 to 0.8, more preferably in the range from 0.20 to 0.7 and even more preferably in the range from 0.25 to 0.6.

In another preferred embodiment, the RM mixture comprises one or more additional RMs, preferably selected from achiral RMs, which are different from formula I, CRM1 to CRM3 and their subformulae. Preferably the RM mixture comprises one or more additional RMs selected from RMs having only one polymerisable functional group (monoreactive RMs), and/or one or more additional RMs having two or more polymerisable functional groups (di- or multireactive RMs).

The additional di- or multireactive RMs are preferably selected of formula DRM

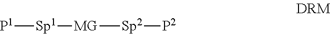

wherein
P¹, P² independently of each other denote a polymerisable group,
Sp¹, Sp² independently of each other are a spacer group or a single bond, and
MG is a rod-shaped mesogenic group, which is preferably selected of formula MG

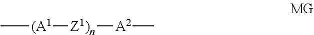

wherein
A¹ and A² denote, in case of multiple occurrence independently of one another, an aromatic or alicyclic group, which optionally contains one or more heteroatoms selected from N, O and S, and is optionally mono- or polysubstituted by L,
L is P-Sp-, F, Cl, Br, I, —CN, —NO₂, —NCO, —NCS, —OCN, —SCN, —C(═O)NR^{x}R^{y}, —C(═O)OR^{x}, —C(═O)R^{x}, —NR^{x}R^{y}, —OH, —SF₅, optionally substituted silyl, aryl or heteroaryl with 1 to 12, preferably 1 to 6 C atoms, and straight chain or branched alkyl, alkoxy, alkylcarbonyl, alkoxycarbonyl, alkylcarbonyloxy or alkoxycarbonyloxy with 1 to 12, preferably 1 to 6 C atoms, wherein one or more H atoms are optionally replaced by F or Cl,
R^{x} and R^{y} independently of each other denote H or alkyl with 1 to 12 C-atoms,
Z¹ denotes, in case of multiple occurrence independently of one another, —O—, —S—, —CO—, —COO—, —OCO—, —S—CO—, —CO—S—, —O—COO—, —CO—NR⁰⁰—, —NR⁰⁰—CO—, —NR⁰⁰—CO—NR⁰⁰⁰, —NR⁰⁰—CO—O—, —O—CO—NR⁰⁰—, —OCH₂—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CR⁰⁰—, —CY¹═CY²—, —C≡C—, —CH═CH—COO—, —OCO—CH═CH— or a single bond, preferably —COO—, —OCO— or a single bond,
Y¹ and Y² independently of each other denote H, F, Cl or CN,
n is 1, 2, 3 or 4, preferably 1 or 2, most preferably 2,
n1 is an integer from 1 to 10, preferably 1, 2, 3 or 4.

Preferred groups A¹ and A² include, without limitation, furan, pyrrol, thiophene, oxazole, thiazole, thiadiazole, imidazole, phenylene, cyclohexylene, bicyclooctylene, cyclohexenylene, pyridine, pyrimidine, pyrazine, azulene, indane, fluorene, naphthalene, tetrahydronaphthalene, anthracene, phenanthrene and dithienothiophene, all of which are unsubstituted or substituted by 1, 2, 3 or 4 groups L as defined above.

Particular preferred groups A¹ and A² are selected from 1,4-phenylene, pyridine-2,5-diyl, pyrimidine-2,5-diyl, thiophene-2,5-diyl, naphthalene-2,6-diyl, 1,2,3,4-tetrahydro-naphthalene-2,6-diyl, indane-2,5-diyl, bicyclooctylene or 1,4-cyclohexylene wherein one or two non-adjacent CH₂ groups are optionally replaced by O and/or S, wherein these groups are unsubstituted or substituted by 1, 2, 3 or 4 groups L as defined above.

Preferred RMs of formula DRM are selected of formula DRMa

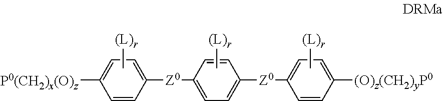

wherein
P⁰ is, in case of multiple occurrence independently of one another, a polymerisable group, preferably an acryl, methacryl, oxetane, epoxy, vinyl, heptadiene, vinyloxy, propenyl ether or styrene group,
Z⁰ is —COO—, —OCO—, —CH₂CH₂—, —CF₂O—, —OCF₂—, —C≡C—, —CH═CH—, —OCO—CH═CH—, —CH═CH—COO—, or a single bond,
L has on each occurrence identically or differently one of the meanings given for L¹ in formula I, and is preferably, in case of multiple occurrence independently of one another, selected from F, Cl, CN or optionally halogenated alkyl, alkoxy, alkylcarbonyl, alkoxycarbonyl, alkylcarbonyloxy or alkoxycarbonyloxy with 1 to 5 C atoms,
r is 0, 1, 2, 3 or 4,
x and y are independently of each other 0 or identical or different integers from 1 to 12,
z is 0 or 1, with z being 0 if the adjacent x or y is 0.

Very preferred RMs of formula DRM are selected from the following formulae:

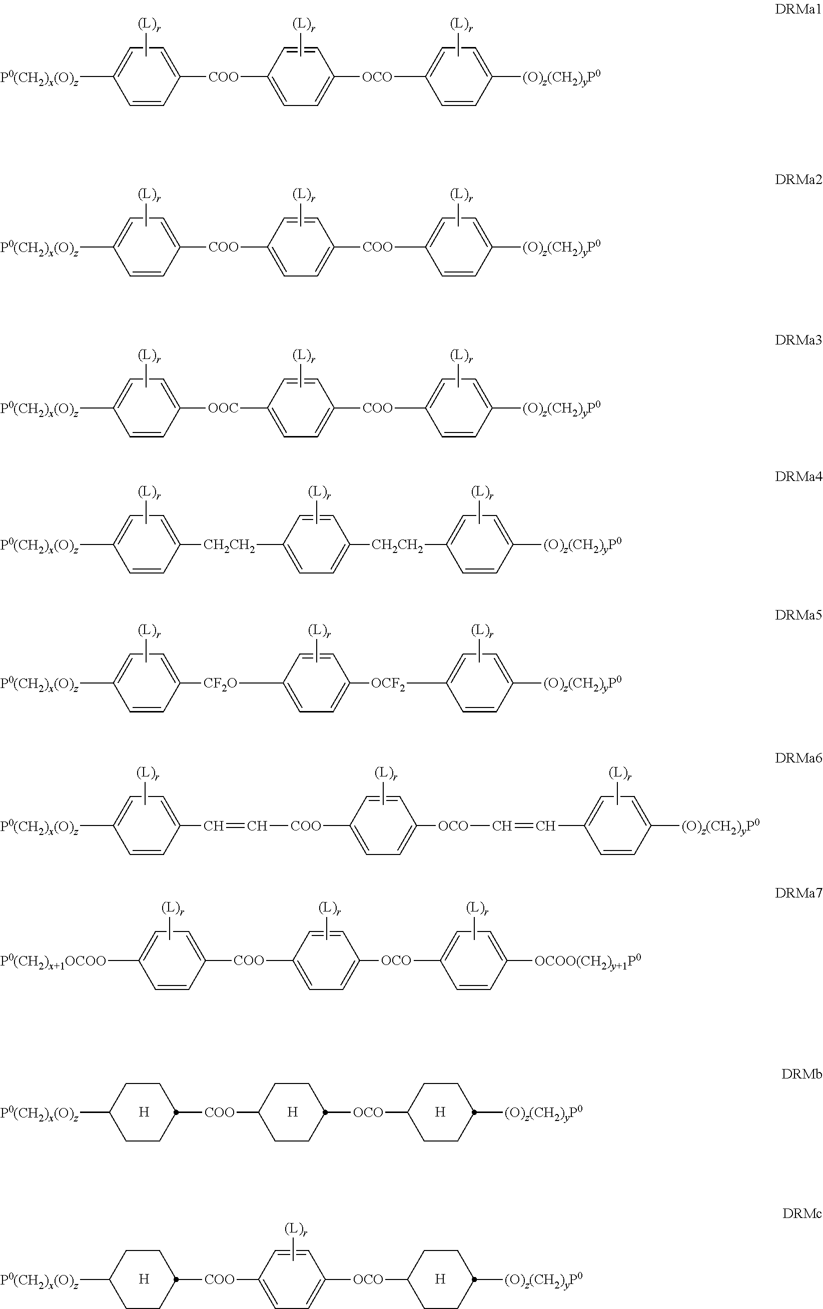

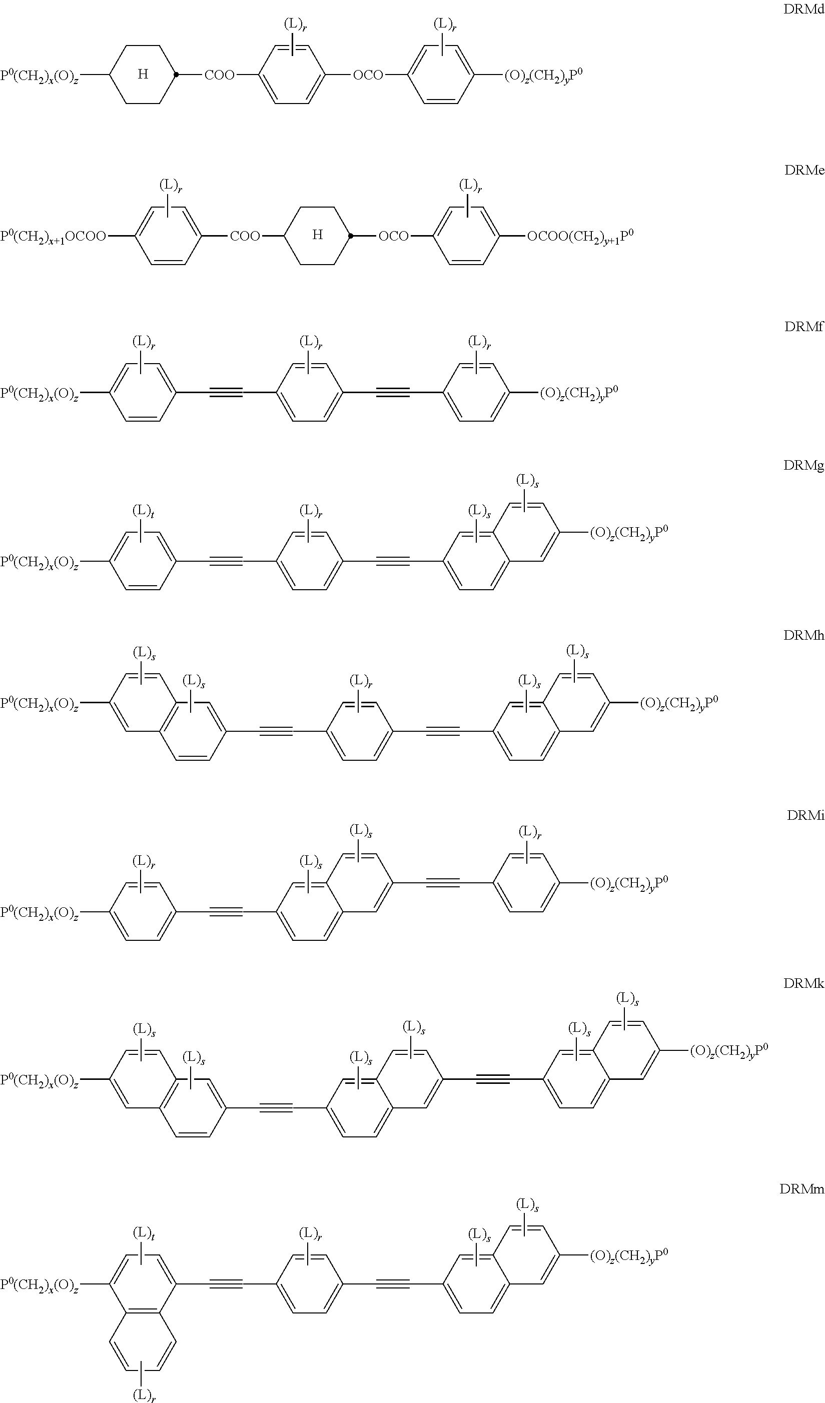

wherein P⁰, L, r, x, y and z are as defined in formula DRMa, s is 0, 1, 2 or 3 and t is 0, 1 or 2.

Especially preferred are compounds of formula DRMa1, DRMa2 and DRMa3, in particular those of formula DRMa1.

Further preferred are compounds of formula DRMf, DRMg, DRMh, DRMi, DRMk and DRMm.

In another preferred embodiment the RM mixture comprises, in addition to the compounds of formula I, one or more monoreactive RMs. These additional monoreactive RMs are preferably selected from formula MRM:

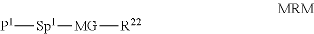

wherein P¹, Sp¹ and MG have the meanings given in formula DRM,
R²² denotes P-Sp-, F, Cl, Br, I, —CN, —NO₂, —NCO, —NCS, —OCN, —SCN, —C(═O)NR^{x}R^{y}, —C(═O)X, —C(═O)OR^{x}, —C(═O)R^{Y}, —NR^{x}R^{y}, —OH, —SF₅, optionally substituted silyl, straight chain or branched alkyl, alkoxy, alkylcarbonyl, alkoxycarbonyl, alkylcarbonyloxy or alkoxycarbonyloxy with 1 to 12, preferably 1 to 6 C atoms, wherein one or more H atoms are optionally replaced by F or Cl,
X is halogen, preferably F or Cl, and
R^{x} and R^{y} are independently of each other H or alkyl with 1 to 12 C-atoms.

Preferably the RMs of formula MRM are selected from the following formulae.

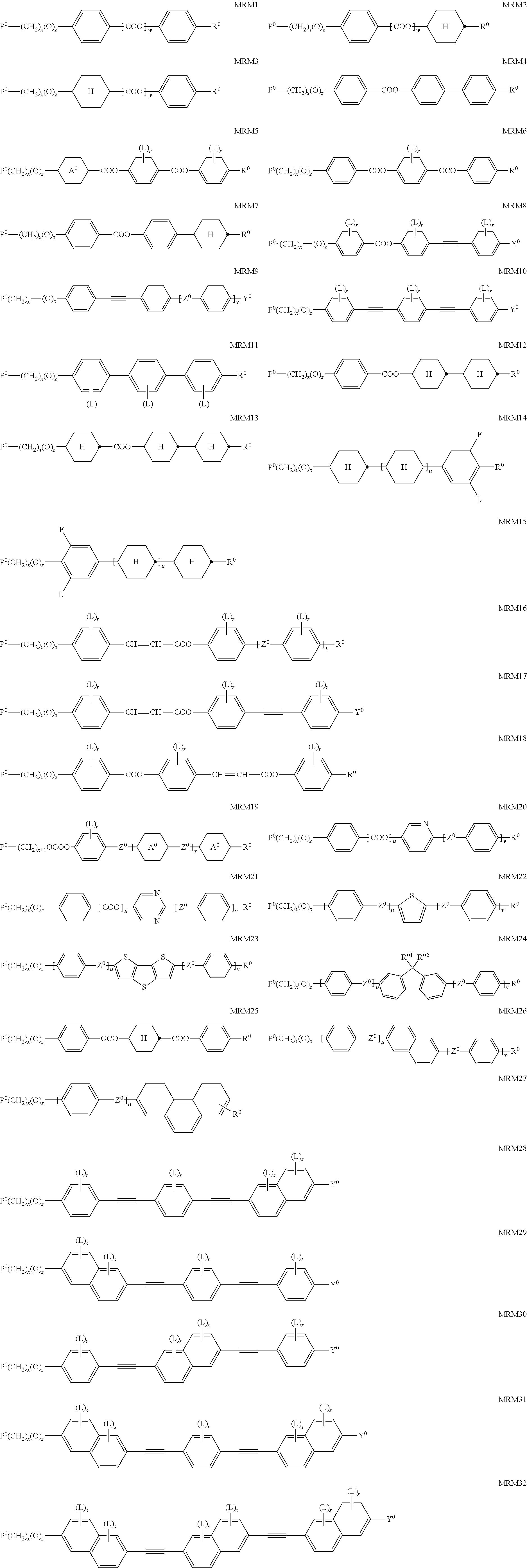

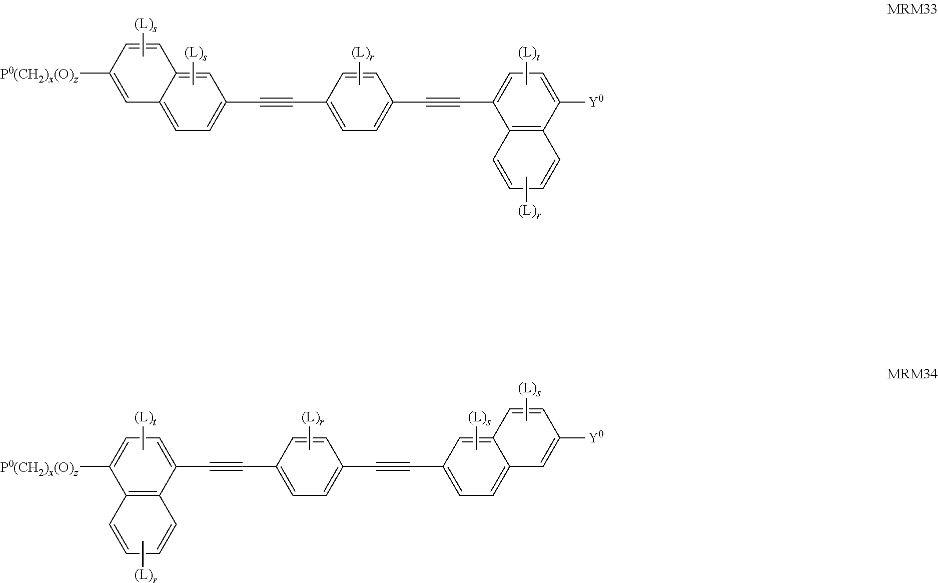

wherein P⁰, L, r, x, y, z, s and t are as defined in formula DRMa and DRMk,
R⁰, R⁰¹
and R⁰² are each an idependently alkyl, alkoxy, thioalkyl, alkylcarbonyl, alkoxycarbonyl, alkylcarbonyloxy or alkoxycarbonyloxy with 1 or more, preferably 1 to 15 C atoms or denotes Y⁰ or P—(CH₂)_{y}—(O)_{z}—,
X⁰ is —O—, —S—, —CO—, —COO—, —OCO—, —O—COO—, —CO—NR⁰¹—, —NR⁰¹—CO—, —NR⁰¹—CO—NR⁰¹—, —OCH₂—, —CH₂O—, —SCH₂—, —CH₂S—, —CF₂O—, —OCF₂—, —CF₂S—, —SCF₂—, —CF₂CH₂—, —CH₂CF₂—, —CF₂CF₂—, —CH═N—, —N═CH—,
   —N═N—, —CH═CR⁰¹—, —CF═CF—, —C≡C—, —CH═CH—COO—, —OCO—CH═CH— or a single bond
Y⁰ is F, Cl, CN, NO₂, OCH₃, OCN, SCN, SF₅, or mono- oligo- or polyfluorinated alkyl or alkoxy with 1 to 4 C atoms,
Z⁰ is —COO—, —OCO—, —CH₂CH₂—, —CF₂O—, —OCF₂—, —CH═CH—, —OCO—CH═CH—, —CH═CH—COO—, or a single bond,
A⁰ is, in case of multiple occurrence independently of one another, 1,4-phenylene that is unsubstituted or substituted with 1, 2, 3 or 4 groups L, or trans-1,4-cyclohexylene,
R^{01,02} are independently of each other H, R⁰ or Y⁰,
u and v are independently of each other 0, 1 or 2,
w is 0 or 1,
   and wherein the benzene and naphthalene rings can additionally be substituted with one or more identical or different groups L.

Especially preferred are compounds of formula MRM1, MRM4, MRM8, MRM9, MRM10, MRM11, MRM28, MRM29, MRM30, MRM31, MRM32, MRM33 and MRM34, in particular those of formula MRM8, MRM9, MRM10, MRM28, MRM29, MRM30, MRM31, MRM32, MRM33 and MRM34.

In formulae DRM, MRM and their preferred subformulae, L is preferably selected from F, Cl, CN, NO₂ or straight chain or branched alkyl, alkoxy, alkylcarbonyl, alkoxycarbonyl, alkylcarbonyloxy or alkoxycarbonyloxy with 1 to 12 C atoms, wherein the alkyl groups are optionally perfluorinated, or P-Sp-.

Very preferably L is selected from F, Cl, CN, NO₂, CH₃, C₂H₅, C(CH₃)₃, CH(CH₃)₂, CH₂CH(CH₃)C₂H₅, OCH₃, OC₂H₅, COCH₃, COC₂H₅, COOCH₃, COOC₂H₅, CF₃, OCF₃, OCHF₂, OC₂F₅ or P-Sp-, in particular F, Cl, CN, CH₃, C₂H₅, C(CH₃)₃, CH(CH₃)₂, OCH₃, COCH₃ or OCF₃, most preferably F, Cl, CH₃, C(CH₃)₃, OCH₃ or COCH₃, or P-Sp-.

If the RM mixture contains additional di- or multireactive RMs different from formula I, preferably selected from formula DRM and its subformulae, their concentration in the RM mixture is preferably from 1 to 50%, very preferably from 1 to 20%.

If the RM mixture contains additional monoreactive RMs different from formula I, preferably selected from formula MRM, their concentration in the RM mixture is preferably from 1 to 30%, very preferably from 1 to 20%.

In another preferred embodiment of the present invention the RM mixture does not contain any additional RMs other than those of formula I.

In another preferred embodiment the RM mixture according to the present invention, in addition to the polymerisable compounds of formula I or its subformulae, comprises one or more chiral isomerisable compounds, preferably selected from chiral photoisomerisable compounds.

The chiral isomerisable compounds can be polymerisable or not polymerisable. They can be non-mesogenic compounds or mesogenic compounds. If the chiral isomerisable compounds are polymerisable they can be monoreactive or multireactive.

In a preferred embodiment the RM mixture according to the present invention comprises one or more chiral isomerisable compounds which are polymerisable.

In another preferred embodiment the RM mixture according to the present invention contains exactly one chiral isomerisable compound.

Further preferably the RM mixture contains only chiral isomerisable compounds which are polymerisable, preferably selected from mono- or direactive chiral isomerisable compounds.

Further preferably the RM mixture does not contain a chiral compound which does not contain an isomerisable group, in particular does not contain a photoisomerisable group.

In another preferred embodiment the RM mixture according to the present invention does not contain any other chiral compounds in addition to the chiral isomerisable compound(s).

Suitable polymerisable chiral isomerisable compounds preferably comprise one or more ring elements, linked together by a direct bond or via a linking group and, where two of these ring elements optionally may be linked to each other, either directly or via a linking group, which may be identical to or different from the linking group mentioned. The ring elements are preferably selected from the group of four-, five-, six- or seven-, preferably of five- or six-, membered rings.

Preferred chiral isomerisable compounds are selected of formula I*:

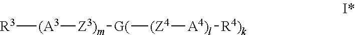

wherein the individual radicals, independently of each other and on each occurrence identically or differently, have the following meanings
R³, R⁴ H, F, Cl, CN, P-Sp- or an alkyl radical with up to 25 C atoms which may be unsubstituted, mono- or polysubstituted by halogen or CN, it being also possible for one or more non-adjacent CH₂ groups to be replaced, in each case independently from one another, by —O—, —S—, —NH—, —N(CH₃)—, —CO—, —COO— —OCO—, —OCO—O—, —S—CO—, —CO—S— or —C≡C— in such a manner that oxygen atoms are not linked directly to one another,
P a polymerisable group,
Sp a spacer group or a single bond,
Z³, Z⁴ —CO—O—, —O—CO—, —CH₂CH₂—, —OCH₂—, —CH₂O—, —CH═CH—, —CH═CH—CO—O—, —O—CO—CH═CH—, —CH═C(CN)—CO—O—, —O—CO—C(CN)═CH—, —N═N—, —CH═N—, —N═CH—, —C≡C—, or a single bond,
A³, A⁴ an alicyclic, heterocyclic, aromatic or heteroaromatic group with 4 to 20 ring atoms, which is monocyclic or polycyclic and which is optionally substituted by one or more groups L or P-Sp-,
G a chiral group,
L F, Cl, —CN, —SCN, P-Sp-, or straight chain, branched or cyclic alkyl having 1 to 25 C atoms, wherein one or more non-adjacent CH₂-groups are optionally replaced by —O—, —S—, —CO—, —CO—O—, —O—CO—, —O—CO—O—, CR⁰═CR⁰⁰—, —C≡C—,

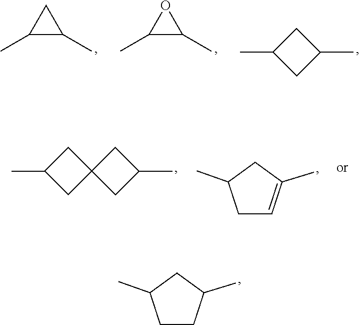

in such a manner that O- and/or S-atoms are not directly connected with each other, and wherein one or more H atoms are each optionally replaced by P-Sp-, F or Cl, or two substituents L that are connected to directly adjacent C atoms may also form a cycloalkyl or cycloalkenyl group with 5, 6, 7 or 8 C atoms,
m, l independently of each other 0, 1, 2 or 3,
k 0, 1 or 2,
   wherein the compound contains at least one isomerisable group, which is preferably a photoisomerisable group.

In the compounds of formula I* and its subformulae as described above and below, if R³ or R⁴ is an alkyl or alkoxy radical, i.e. where the terminal CH₂ group is replaced by —O—, this may be straight-chain or branched. It is preferably straight-chain, has 2, 3, 4, 5, 6, 7 or 8 carbon atoms and accordingly is preferably ethyl, propyl, butyl, pentyl, hexyl, heptyl, octyl, ethoxy, propoxy, butoxy, pentoxy, hexoxy, heptoxy, or octoxy, furthermore methyl, nonyl, decyl, undecyl, dodecyl, tridecyl, tetradecyl, pentadecyl, methoxy, nonoxy, decoxy, undecoxy, dodecoxy, tridecoxy or tetradecoxy, for example.

Oxaalkyl, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

Preferred compounds of formula I* and its subformulae are those wherein at least one of R³ and R⁴, preferably both R³ and R⁴, denote P-Sp-.

Further preferred compounds of formula I* and its subformulae are those wherein at least one of R³ and R⁴, preferably both R³ and R⁴, is different from P-Sp-, and preferably denotes alkyl or alkoxy with 1 to 12, more preferably 1 to C atoms, and one of R³ and R⁴ may also denote F, Cl or CN.

Further preferred compounds of formula I* and its subformulae are those wherein A³ and A⁴ are selected from the group consisting of 1,4-phenylene, 1,3-phenylene, naphthalene-1,4-diyl, naphthalene-2,6-diyl, phenanthrene-2,7-diyl, 9,10-dihydro-phenanthrene-2,7-diyl, anthracene-2,7-diyl, anthracene-9,10-diyl, fluorene-2,7-diyl, dibenzothiophene-2,7-diyl, dibenzofuran-2,7-diyl, benzo[1,2-b:4,5-b′]dithiophene-2,5-diyl, indole-4,7-diyl, benzothiophene-4,7-diyl, coumarine, flavone, where, in addition, one or more CH groups in these groups may be replaced by N, cyclohexane-1,4-diyl, in which, in addition, one or more non-adjacent CH₂ groups may be replaced by O and/or S, 1,4-cyclohexenylene, bicycle-[1.1.1]pentane-1,3-diyl, bicyclo[2.2.2]octane-1,4-diyl, spiro[3.3]heptane-2,6-diyl, piperidine-1,4-diyl, decahydronaphthalene-2,6-diyl, 1,2,3,4-tetrahydronaphthalene-2,6-diyl, indane-2,5-diyl, octahydro-4,7-methanoindane-2,5-diyl, 2-benzylidene-1-indanone, chalcone, chromone and pentalenone, all of which are optionally substituted by one or more groups L or P-Sp-.

Very preferred compounds of formula I* and its subformulae are those wherein A³ and A⁴ are selected from the group consisting of 1,4-phenylene, naphthalene-1,4-diyl, naphthalene 2,6-diyl, 1,4-cyclohexylene in which, in addition, one or two non-adjacent CH₂ groups may be replaced by O and/or S, 1,4-cyclohexenylene, 1,4-bicyclo(2,2,2)octylene, piperidine-1,4-diyl, naphthalene-2,6-diyl, decahydronaphthalene-2,6-diyl, or 1,2,3,4-tetrahydro-naphthalene-2,6-diyl, very preferably 1,4-phenylene or 1,4-cyclohexylene, all of which are optionally substituted by one or more groups L or P-Sp.

Further preferred compounds of formula I* and its subformulae are those wherein Z³ and Z⁴ independently of each other denote —CO—O—, —O—CO— or a single bond.

Further preferred compounds of formula I* and its subformulae are those wherein L is selected from F, Cl, CN, CH₃, C₂H₅, OCH₃, OC₂H₅, COCH₃, COC₂H₅, CF₃, OCF₃, P-Sp-, in particular F, Cl, CN, CH₃, C₂H₅, OCH₃, COCH₃ or OCF₃, most preferably F, CH₃, OCH₃ or COCH₃.

Further preferred compounds of formula I* and its subformulae are those wherein P is selected from the group consisting of vinyloxy, acrylate, methacrylate, fluoroacrylate, chloroacrylate, oxetane and epoxide, very preferably from acrylate and methacrylate, most preferably acrylate.

Further preferred compounds of formula I* and its subformulae are those wherein Sp denotes a single bond or —(CH₂)ₚ₁—, —O—(CH₂)ₚ₁—, —O—CO—(CH₂)ₚ₁, or —CO—O—(CH₂)ₚ₁, wherein p1 is an integer from 2 to 10, preferably 2, 3, 4, 5 or 6, and, if Sp is —O—(CH₂)ₚ₁—, —O—CO—(CH₂)ₚ₁ or —CO—O—(CH₂)ₚ₁ the O-atom or CO-group, respectively, is linked to the benzene ring.

Further preferred compounds of formula I* and its subformulae are those wherein all polymerisable groups P that are present in the compound have the same meaning, and very preferably denote acrylate or methacrylate, most preferably acrylate.

Further preferred compounds of formula I* and its subformulae are those which contain one, two, three or four groups P-Sp, very preferably two or three groups P-Sp.

Further preferred compounds of formula I* and its subformulae are those wherein at least one group Sp is a single bond.

Further preferred compounds of formula I* and its subformulae are those wherein at least one group Sp is a single bond and at least one group Sp is different from a single bond.

Further preferred compounds of formula I* and its subformulae are those wherein at least one group Sp is different from a single bond, and is selected from —(CH₂)ₚ₁—, —O—(CH₂)ₚ₁—, —O—CO—(CH₂)ₚ₁, or —CO—O—(CH₂)ₚ₁, wherein p1 is an integer from 2 to 10, preferably 2, 3, 4, 5 or 6, and, if Sp is —O—(CH₂)ₚ₁—, —O—CO—(CH₂)ₚ₁ or —CO—O—(CH₂)ₚ₁ the O-atom or CO-group, respectively, is linked to the benzene ring.

In the event that R^{a} or R^{b} is a group of formula P-Sp-, the spacer groups on each side of the mesogenic core may be identical or different.

In the compounds of formula I* and its subformulae as described above and below, m and l are preferably 0 or 1.

In the compounds of formula I* and its subformulae as described above and below, q is preferably 0 or 1, very preferably 0.

Of the compounds of formula I*, the following are especially preferred:

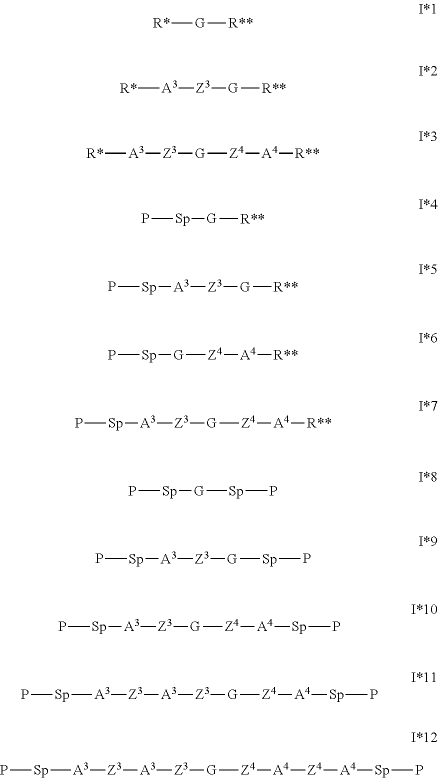

wherein P, Sp, A³, A⁴, Z³, Z⁴ and G have the meanings given for formula I* or one of their preferred meanings as described above and below, R* has one of the meanings of R³ which is different from P-Sp-, and R** has one of the meanings of R⁴ which is different from P-Sp-.

Of these preferred compounds, particularly preferred are those of formula I*8 to I*10, very particularly preferred those of formula I*8.

A smaller group of particularly preferred compounds of the formulae I*1 to I*10 is listed below. For reasons of simplicity, Phe is 1,4-phenylene which is optionally substituted in 2- and/or 3-position with L, and Cyc is 1,4-cyclohexylene.

Particularly preferred compounds of the formula I*2, I3, I*5, I*6, I*7, I*9 and I*10 are those of the following formulae:

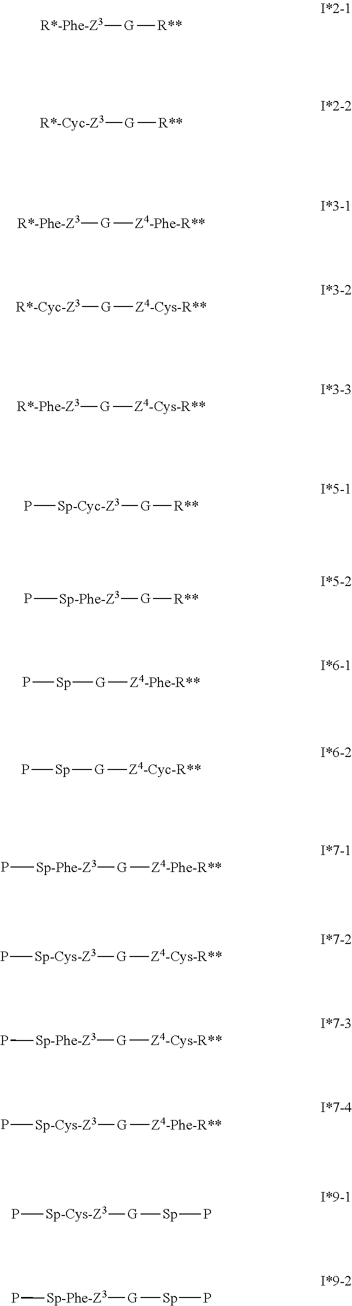

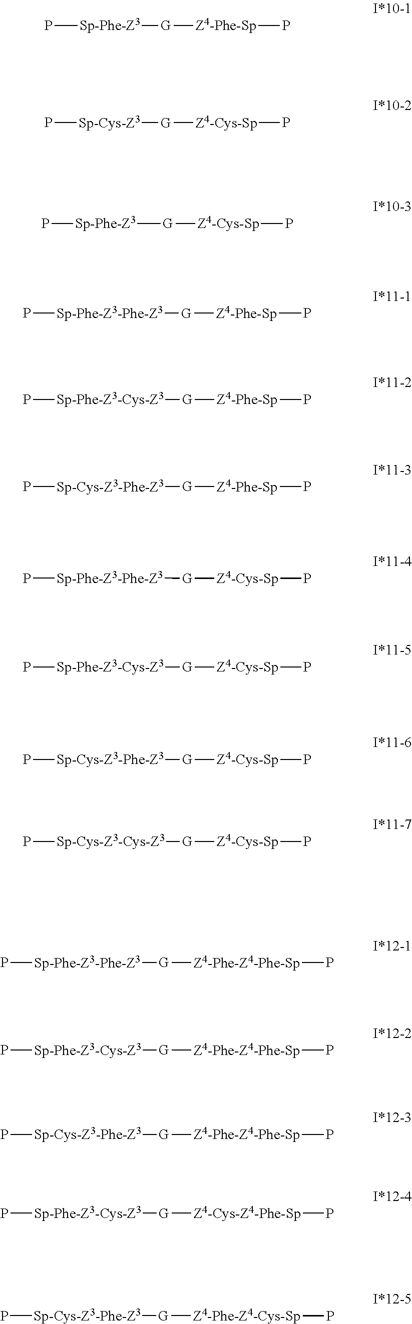

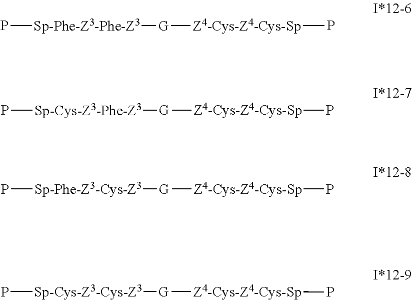

wherein P, Sp, Z³, Z⁴ and G have the meanings given for formula I* or one of their preferred meanings as described above and below, R* has one of the meanings of R³ in formula I* which is different from P-Sp-, and R** has one of the meanings of R⁴ in formula I* which is different from P-Sp-.

Preferably in the compounds of formulae I*2-1 to I*7-4 R* and R** are independently of each other alkyl or alkoxy with 1 to 12 C atoms, or alkyl or alkoxy with 1 to 12 C atoms and the other is F, Cl or CN. Furthermore -Sp- is preferably alkylene or alkyleneoxy with 1 to 12 C atoms, P is preferably acrylate or methacrylate, and Z³ and Z⁴ are independently of each other denote —CO—O—, —O—CO— —CH═CH—CO—O—, —O—CO—CH═CH—, —CH═C(CN)—CO—O—, —O—CO—C(CN)═CH—, —CH═N—, —N═CH—, —N═N— or a single bond, more preferably —CO—O—, —O—CO— or a single bond.

Preferred compounds of formula I* and its subformulae are those wherein G denotes or contains a photoisomerisable group.

Further preferred compounds of formula I* and its subformulae are those wherein Z³ and/or Z⁴ independently of each other denote —CH═CH—CO—O—, —O—CO—CH═CH—, —CH═C(CN)—CO—O—, —O—CO—C(CN)═CH—, —CH═N—, —N═CH— or —N═N—.

Further preferred compounds of formula I* and its subformulae are those containing an isomerisable group selected from stilbene, (1,2-difluoro-2-phenyl-vinyl)-benzene, cinnamate, a-cyanocinnamate, 4-phenylbut-3-en-2-one, Schiff base, 2-benzyliden-1-indanone, chalcone, coumarin, chromone, pentalenone or azobenzene.

Further preferred compounds of formula I* and its subformulae are those wherein the chiral group G is selected or derived from dianhydrohexitol, preferably isosorbide, isomannide or isoidide, 1,1′-bi-2-naphthol (binol), 1,2-diphenyl-1,2-ethanediol (hydrobenzoin), 2-benzylidene-p-menthan-3-one and menthyl cinnamate ((1R,2S,5R)-5-Methyl-2-(1-methylethyl)cyclohexyl (2E)-3-phenyl-2-propenoate).

Very preferred compounds of formula I* and its subformulae are those wherein the chiral group G is selected of formula A:

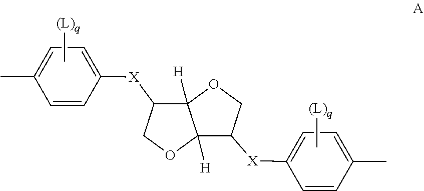

wherein X is —CO—O—, —CH═CH—CO—O—, —CH═C(CN)—CO—O—, in each of which the ester O-atom is linked to the furan ring, or —N═N—, q is 0, 1, 2, 3 or 4, and L has the meaning of formula I* or one of its preferred meanings as given above and below.

Formula A includes the following stereoisomers based on the corresponding dianhydrohexitols:

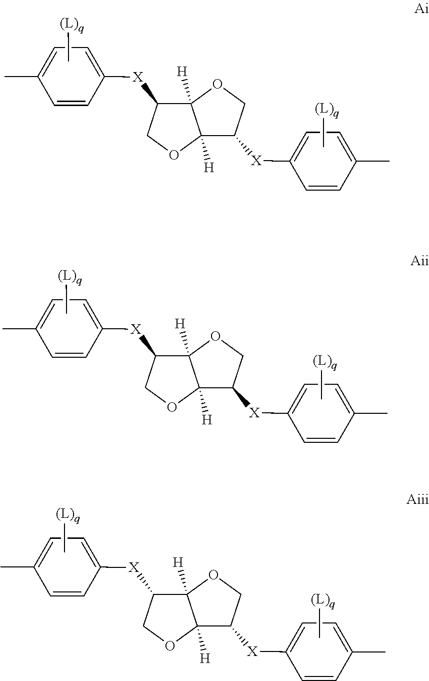

wherein X, L and q have the meanings given in formula A, and wherein Ai is based on isosorbide, Aii is based on isomannide and Aiii is based on isoidide. Especially preferred is Ai.

Further preferred compounds of formula I* and its subformulae are those wherein one or both of Z³ and Z⁴ independently of each other denote —CH═CH—CO—O—, —O—CO—CH═CH—, —CH═C(CN)—CO—O—, —O—CO—C(CN)═CH—, —CH═N—, —N═CH— or —N═N—, and/or wherein G is of formula A, preferably Ai, and X denotes —CH═CH—CO—O—, —CH═C(CN)—CO—O— or —N═N—.

Further preferred compounds of formula I* and its subformulae are those wherein G is of formula A, preferably formula Ai, and X denotes —CH═CH—CO—O—, —CH═C(CN)—CO—O— or —N═N—, very preferably —CH═CH—CO—O—.

Further preferred compounds of formula I* and its subformulae are those wherein the chiral group G is selected from the following formulae

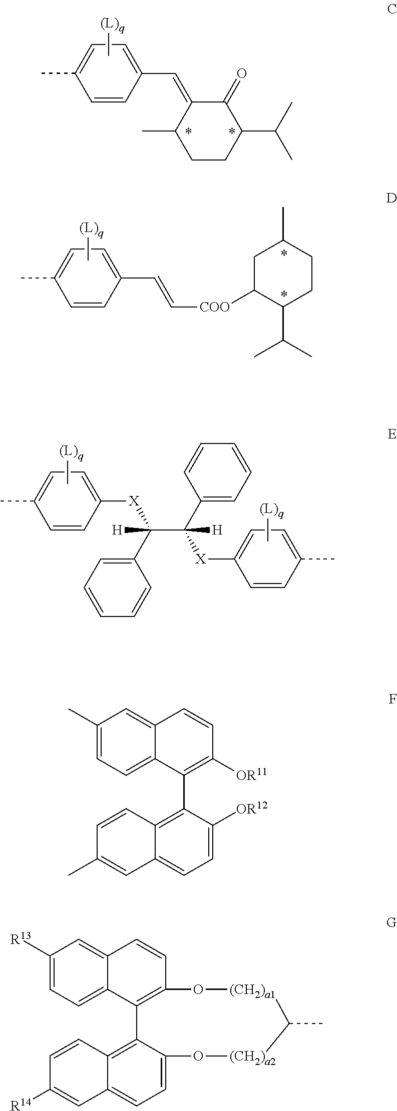

wherein
X, L and q have the meanings given in formula A or one of the preferred meanings as given above and below,
R¹¹ and R¹² independently of each other denote —(Z⁴-A⁴)ᵢ-R⁴ as defined in formula I*, or R¹¹ and R¹² together with the O atoms form a cyclic group or a spirocyclic group which is optionally substituted by a group —(Z⁴-A⁴)ᵢ-R⁴ as defined in formula I*,
R¹³ and R¹⁴ independently of each other denote R³-(A³-Z³)ₘ— as defined in formula I*,
a1 and a2 independently of each other are 0, 1 or 2, and the dashed lines represent a linkage to the adjacent group(s) in formula I*.

Preferred compounds of formula I* are selected from the following formulae:

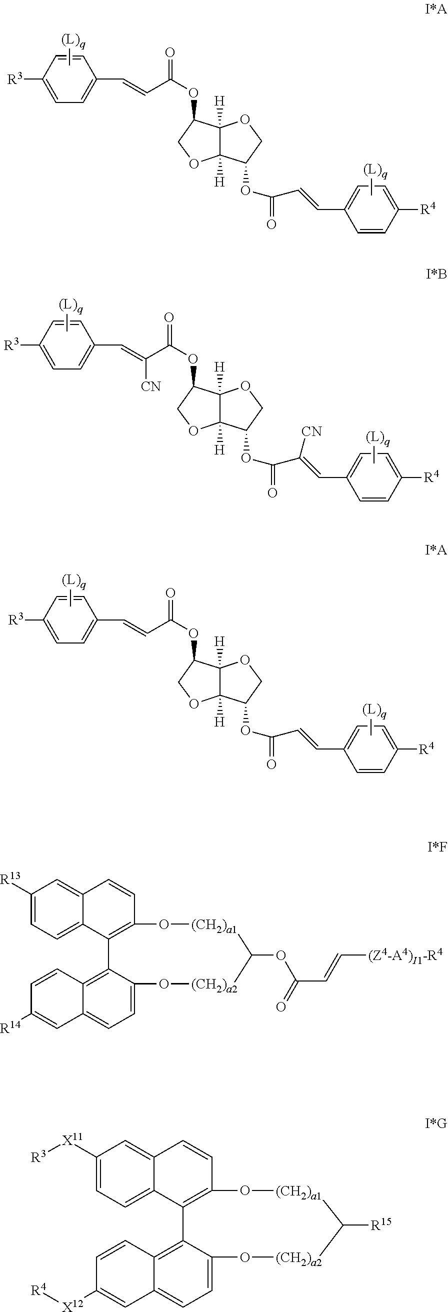

wherein R³, R⁴, Z⁴, A⁴, L and q have the meanings given in formula I* or one of the preferred meanings as given above and below, I1 is 0, 1 or 2, R¹³, R¹⁴, a1 and a2 have the meanings given in formula G or one of the preferred meanings as given above and below, R¹⁵ denotes —(Z⁴-A⁴)ᵢ-R⁴ as defined in formula I* and X¹¹ and X¹² denote —O—CO—CH═CH—.

Very preferred compounds of formula I*A are selected from the following subformulae:

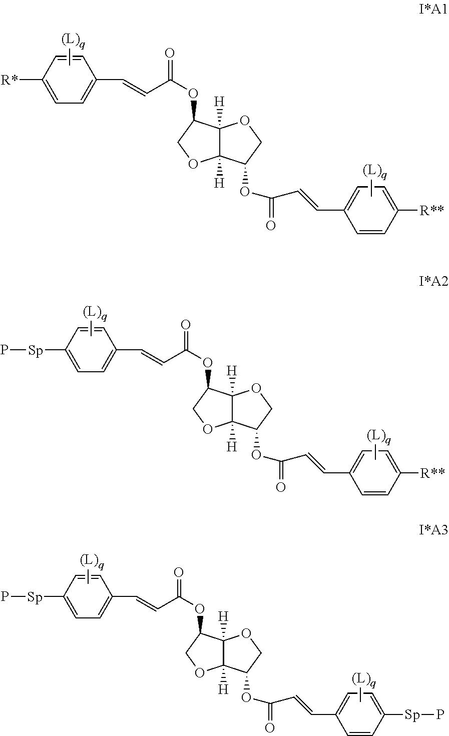

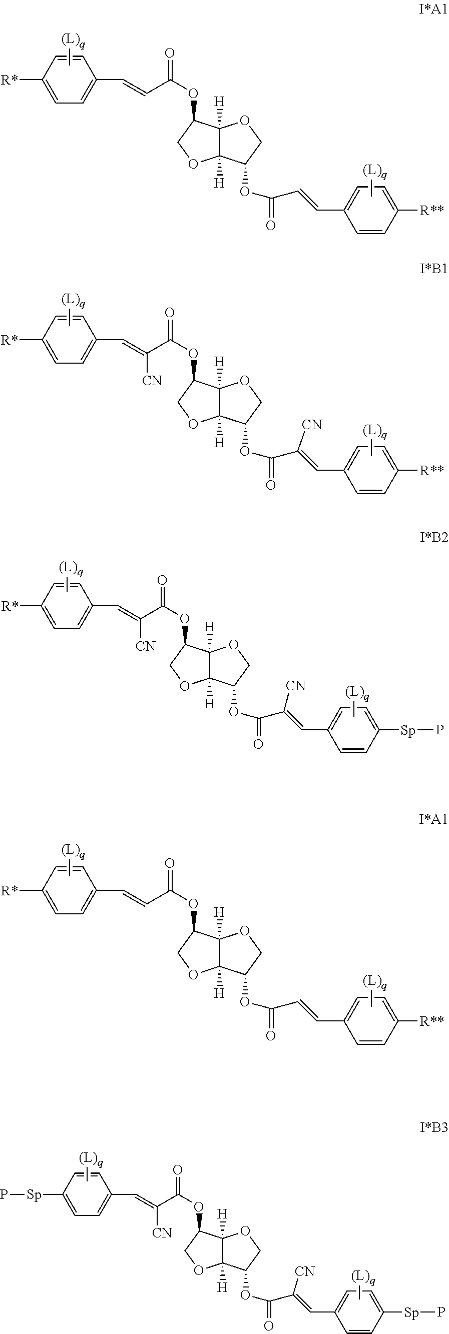

wherein P, Sp, L and q have the meanings given in formula I* or one of the preferred meanings as given above and below, R* has one of the meanings of R³ in formula I* which is different from P-Sp-, and R** has one of the meanings of R⁴ in formula I* which is different from P-Sp-.

Especially preferred are the compounds of formula I*A3.

Further preferred are the stereoisomers of formula I*A, I*B, I*A1, I*A2 and I*A3 wherein the central isosorbide unit is replaced by an isomannide or isoidide unit.

In the compounds of formula I*A, I*B, I*A1, I*A2 and I*A3, P is preferably acrylate or methacrylate, very preferably acrylate, Sp is preferably —O—(CH₂)ₚ₁—, —O—CO—(CH₂)ₚ₁— or —CO—O—(CH₂)ₚ₁—, very preferably —O—(CH₂)ₚ₁—, wherein the O-atom or CO-group, respectively, is linked to the benzene ring, p1 is an integer from 1 to 6, more preferably 2, 3, 4, 5 or 6, and R⁴ is preferably P-Sp-.

Further preferred compounds of formula I* and its subformulae are selected from the following formulae:

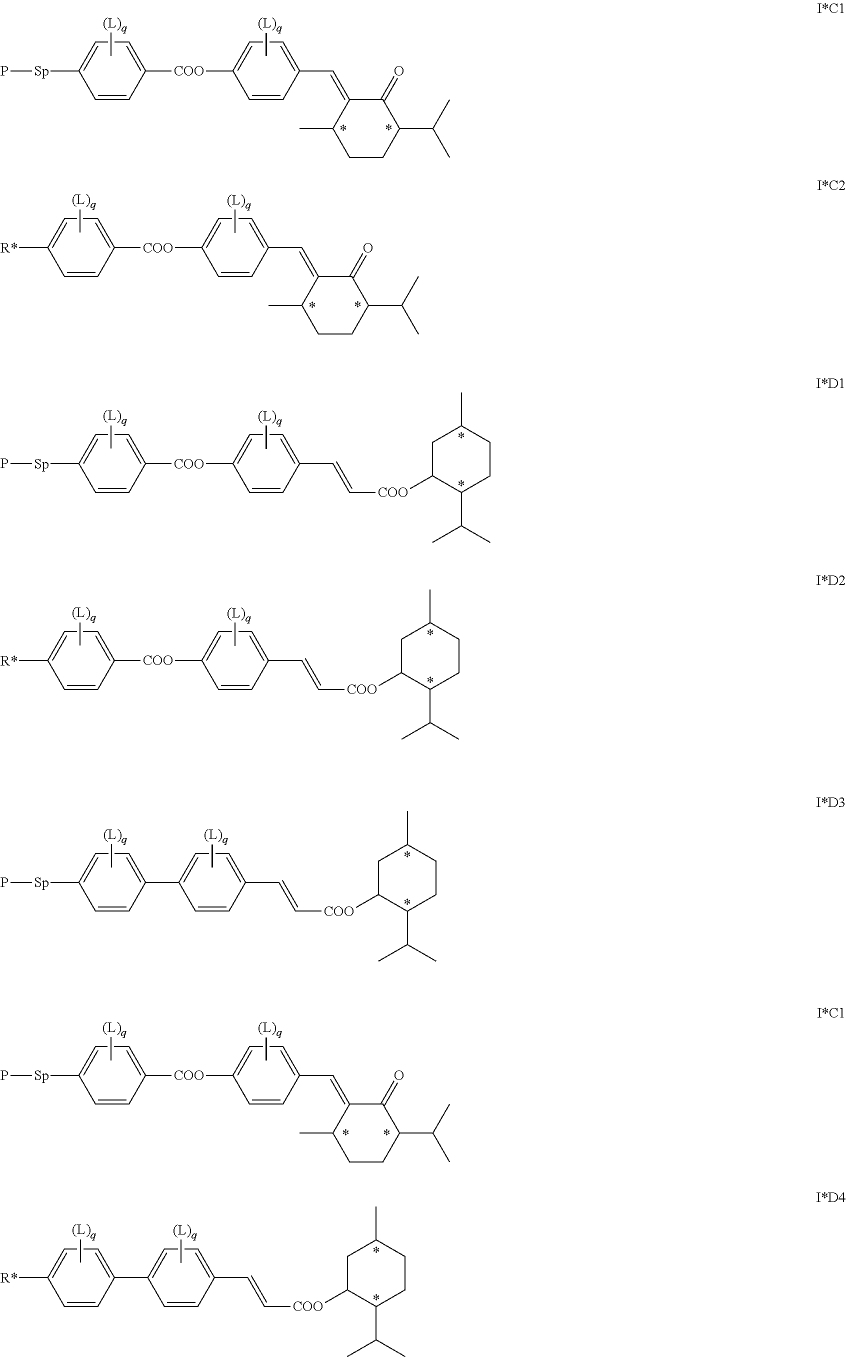

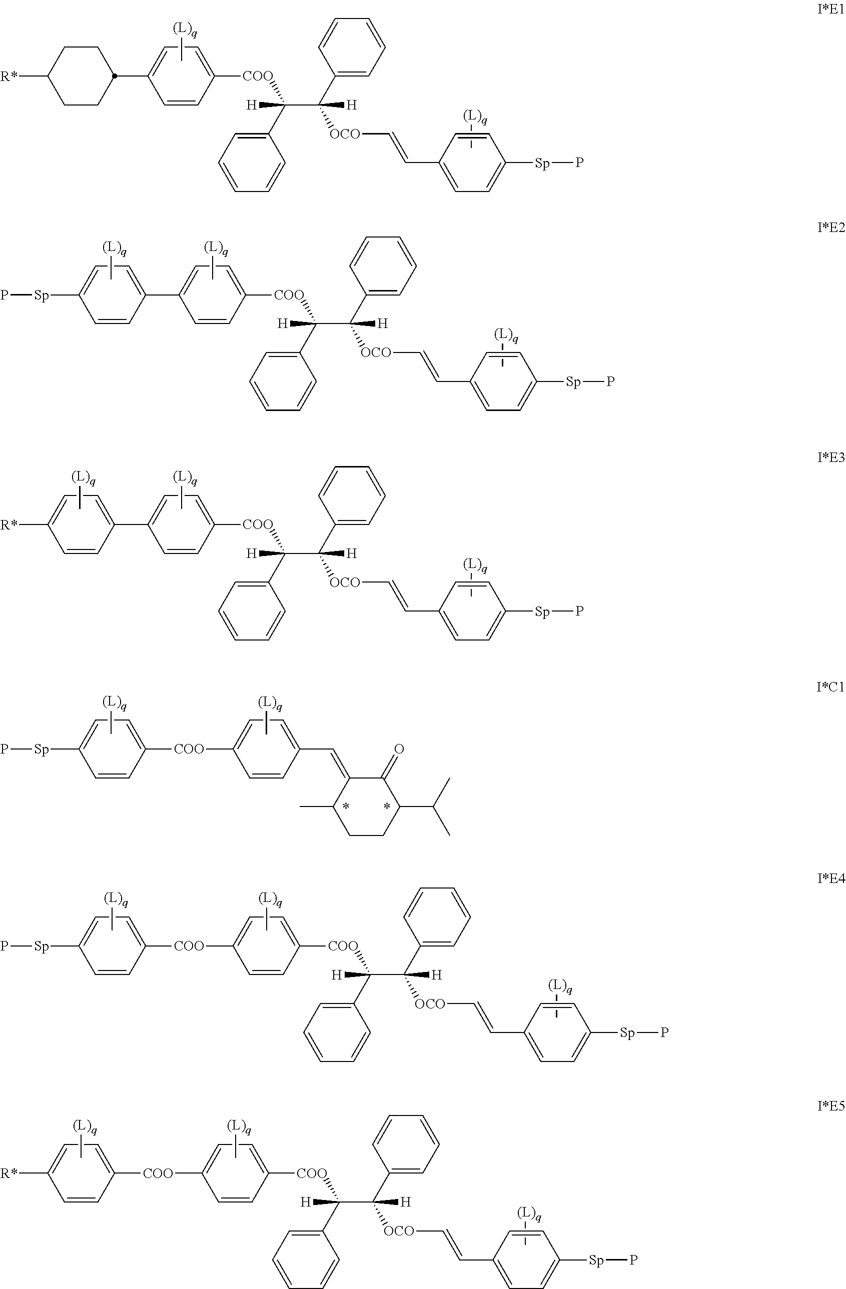

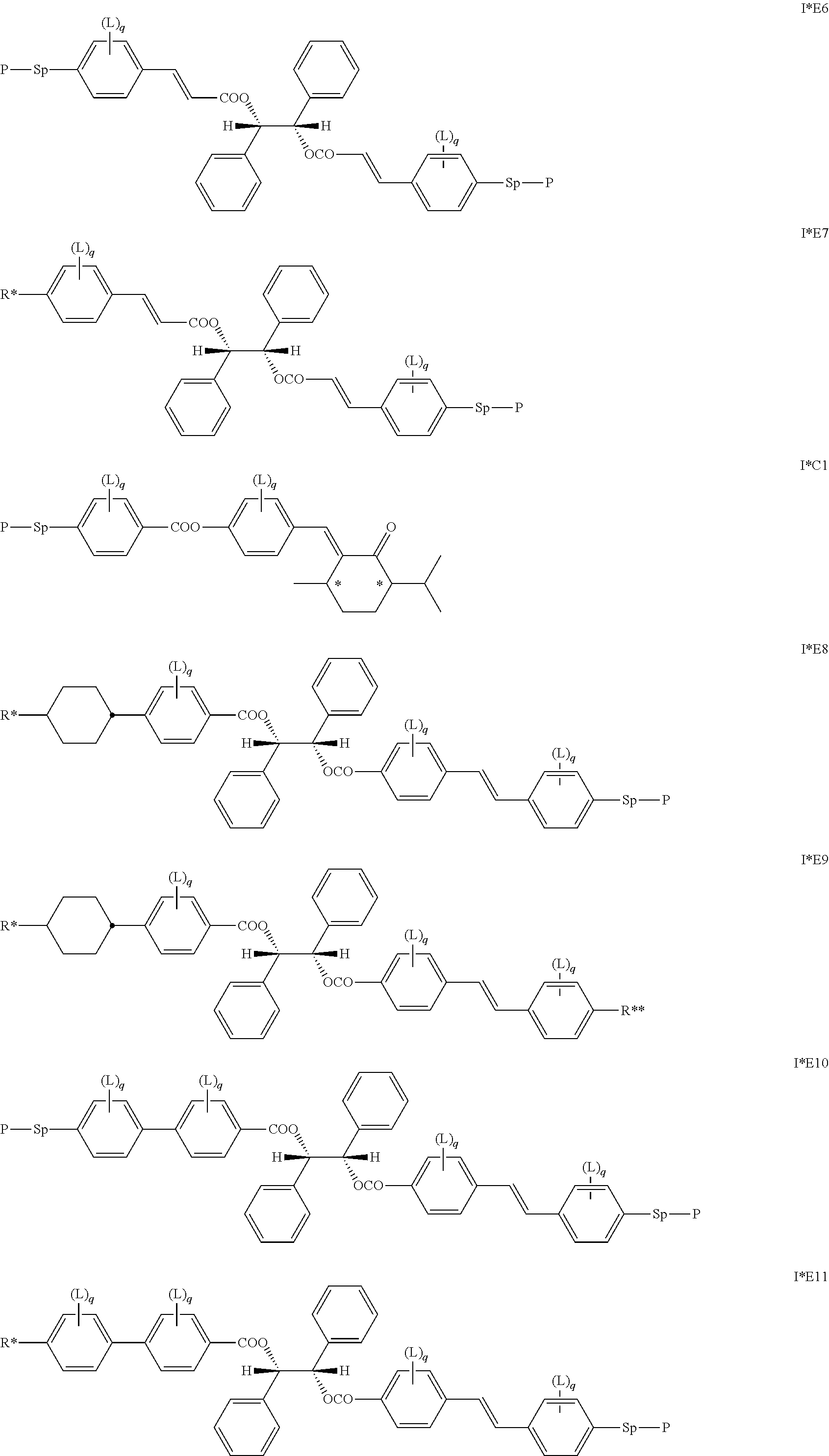

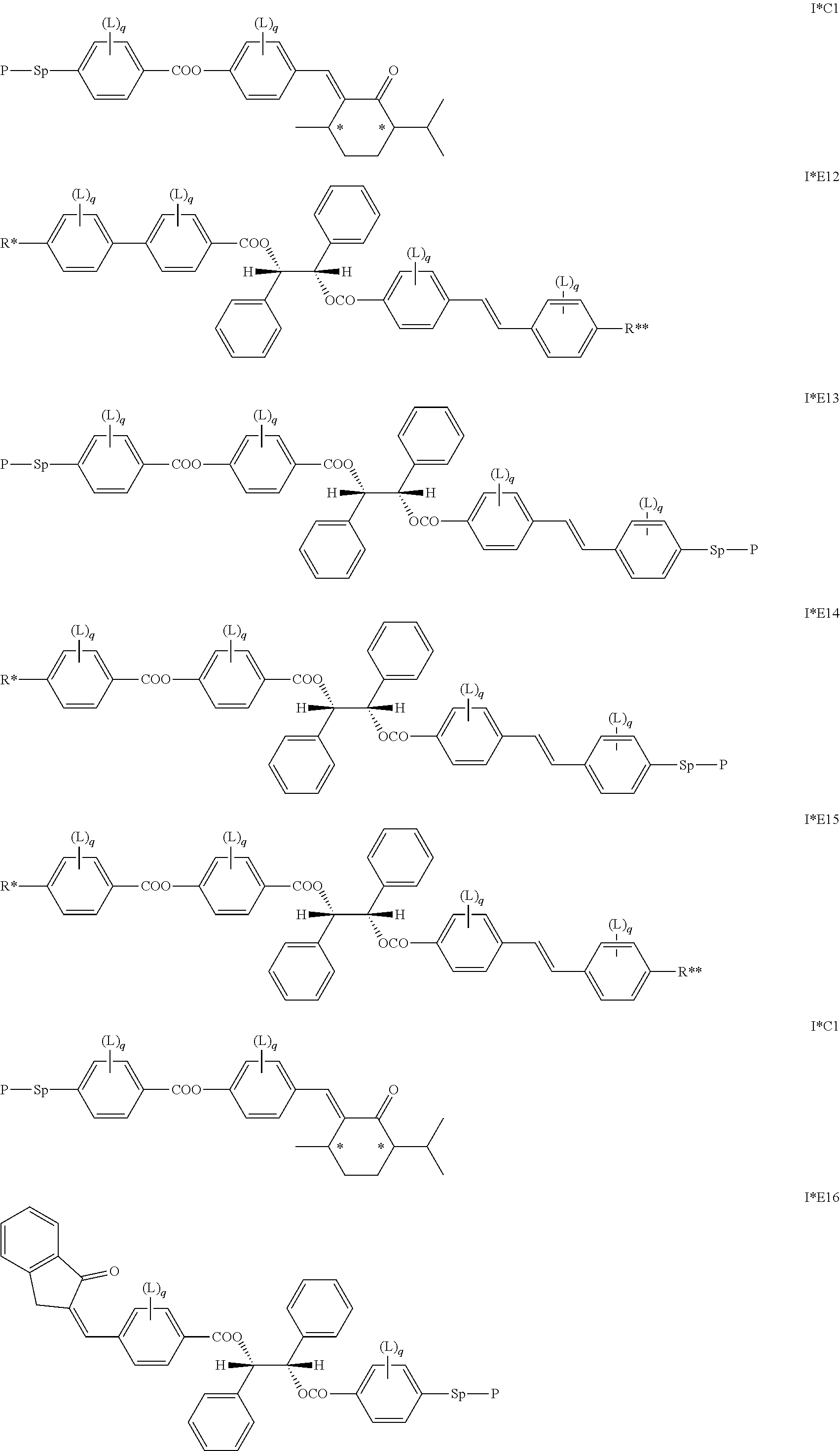

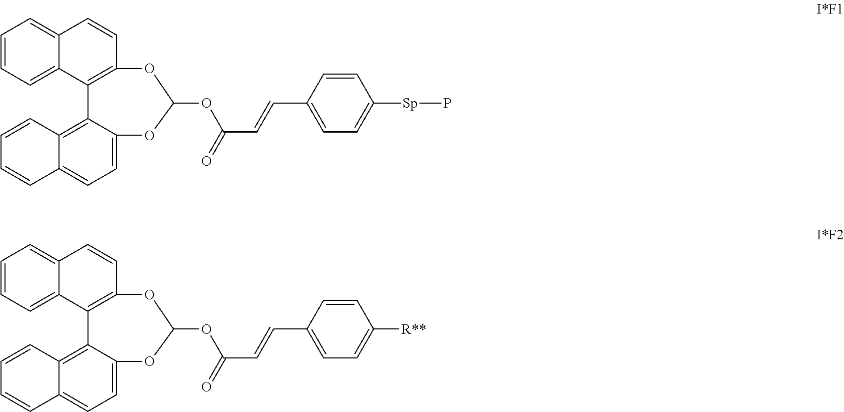

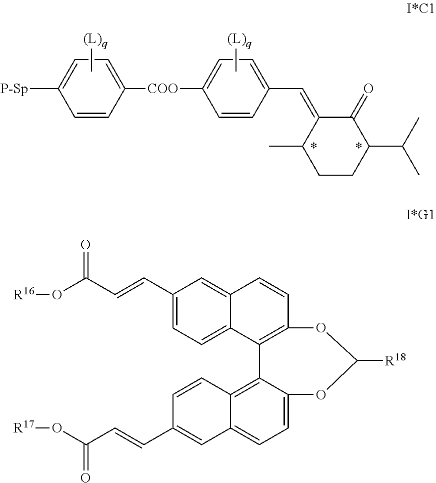

wherein P, Sp, R*, R**, L and q have the meanings given in formula I* and I*A1 or one of the preferred meanings as given above and below, R¹⁶ and R¹⁷ independently of each other denote alkyl with 1 to 12, preferably 1 to 6 C atoms, very preferably methyl, ethyl or propyl, and R¹⁸ denotes P-Sp-, H or alkyl with 1 to 12, preferably 1 to 6 C atoms, very preferably H.

In the compounds of formulae I*C1 to I*G1, P is preferably acrylate or methacrylate, very preferably acrylate, Sp is preferably —O—(CH₂)ₚ₁—, —O—CO—(CH₂)ₚ₁— or —CO—O—(CH₂)ₚ₁—, very preferably —O—(CH₂)ₚ₁—, wherein the O-atom or CO-group, respectively, is linked to the benzene ring, p1 is an integer from 1 to 6, more preferably 2, 3, 4, 5 or 6, R* and R** are preferably, independently of each other, alkyl or alkoxy with 1 to 12, very preferably 1 to 6, C atoms.

The compounds of formula IA* can be prepared for example according to or in analogy to the method described in GB 2314839 A. The compounds of formulae I*E1 to I*E15 can be prepared for example according to or in analogy to the method described in WO 02/40614 A1.

Preferably the utilized chiral isomerisable compounds have each alone or in combination with each other an absolute value of the helical twisting power (|HTPₜₒₜₐₗ|) of 20 μm⁻¹ or more, preferably of 40 μm⁻¹ or more, more preferably in the range of 60 μm⁻¹ or more, most preferably in the range of 80 μm⁻¹ or more to 260 μm⁻¹.

In case the RM mixture contains two or more chiral isomerisable compounds, these compounds may have the same or opposite twist sense.

In a preferred embodiment, the RM mixture contains only one chiral isomerisable compound, very preferably selected from formula I* or its subformulae, which is preferably polymerisable, i.e., which contains at least one group P-Sp-.

In another preferred embodiment the RM mixture does not contain any other chiral compounds than those of formula I*.

Preferably the proportion of the chiral isomerisable compounds, especially those selected from formula I* or its subformulae, in the RM mixture according to the present invention as a whole is in the range from 0.1 to 10% by weight, very preferably in the range from 0.2 to 8.5% by weight, most preferably in the range from 0.5 to 4% by weight.

In another preferred embodiment the RM mixture contains, in addition to the compounds of formula I, one or more chiral compounds which are not isomerisable.

By adding one or more non-isomerisable chiral compounds it is possible to adjust the central wavelength of the reflection band of the RM mixture. In case the RM mixture contains a chiral isomerisable compound, the additional non-isomerisable chiral compound can have the same twist sense or opposite twist sense than the chiral isomerisable compound.

Accordingly the reflection waveband of the RM mixture will be shifted to shorter or longer wavelengths, respectively.

In another preferred embodiment the RM mixture contains one or more, preferably exactly one, chiral isomerisable and polymerisable compound, especially selected from formula I* or its subformulae, and additionally contains one or more, preferably exactly one, polymerisable chiral compound which is not isomerisable, and which very preferably has opposite twist sense than the chiral isomerisable and polymerisable compound, and is preferably selected from formula CRM or its subformulae.

In another preferred embodiment the RM mixture according to the present invention additionally comprises one or more chiral compounds which are not polymerisable and not isomerisable. These chiral compounds may be non-mesogenic compounds or mesogenic compounds.

The further chiral compounds can have the same twist sense or opposite twist sense than the chiral isomerisable compound. Thereby it is possible to shift the reflection waveband of the RM mixture to shorter or longer wavelengths as described above.

Preferred non-polymerisable chiral compounds are selected from the group consisting of compounds of formulae C—I to C—III,

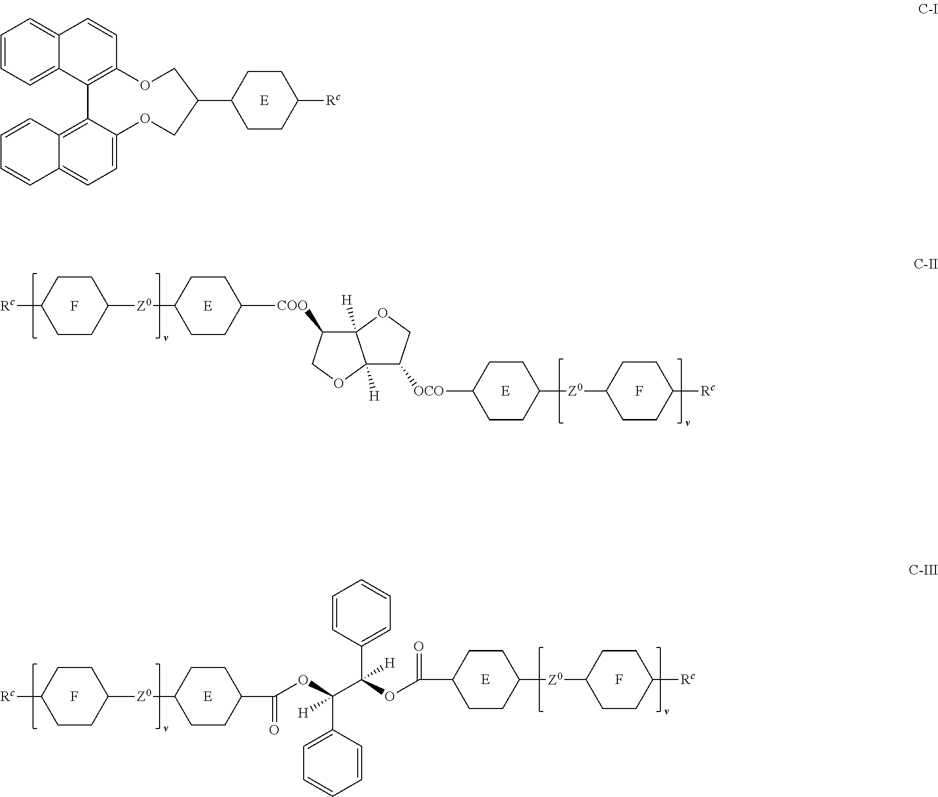

wherein formula C-II and C—III include the respective (S,S) enantiomers, and wherein E and F are each independently 1,4-phenylene or trans-1,4-cyclohexylene, v is 0 or 1, Z⁰ is —COO—, —OCO—, —CH₂CH₂— or a single bond, and R⁰ is alkyl, alkoxy or alkanoyl with 1 to 12 C atoms.

Further preferred are the stereoisomers of formula C-II wherein the central isosorbide unit is replaced by an isomannide or isoidide unit.

The compounds of formula C—I and their synthesis are described in EP1389199 A1. The compounds of formula C-II and their synthesis are described in WO98/00428 A1. The compounds of formula C—III and their synthesis are described in GB2328207 A.

Further preferred additional chiral dopants are e.g. the commercially available R/S-6011, R/S-5011, R/S-4011, R/S-3011, R/S-2011, R/S-1011, R/S-811 and CB-15 (from Merck KGaA, Darmstadt, Germany).

The amount of the non-polymerisable chiral dopants in the RM mixture is preferably from 0.1 to 10%, more preferably from 0.5 to 8% by weight of all solids.

Another object of the invention is an RM formulation comprising an RM mixture as described above and below, and further comprising one or more solvents and/or additives.

The proportion of the RM mixture comprising, preferably consisting of, compounds selected from formula I and its subformulae and optionally from formulae CRM1, CRM2, CRM3, DRM, MRM, I* and their subformulae, in the RM formulation is preferably from 85 to 100%, more preferably from 85 to 99%, very preferably from 90 to 99% of total solids and liquid additives, i.e., excluding the solvents.

In a preferred embodiment the RM mixure or RM formulation additionally comprises one or more additives selected from the group consisting of polymerisation initiators, surfactants, stabilisers, catalysts, sensitizers, inhibitors, chain-transfer agents, co-reacting monomers, reactive thinners, surface-active compounds, lubricating agents, wetting agents, dispersing agents, hydrophobing agents, adhesive agents, flow improvers, degassing or defoaming agents, deaerators, diluents, reactive diluents, auxiliaries, colourants, dyes, pigments and nanoparticles.

In another preferred embodiment the present invention, the RM mixture and/or RM formulation do not contain a compound with at least one CF₃ or CF₂ group (PFAS), and very preferably the RM mixture and/or RM formulation do not contain a compound with a polyfluorinated alkyl or aryl group or a perfuorocarbon group. More preferably the RM mixture and/or RM formulation do not contain a compound with a fluorinated aliphatic C atom, most preferably the RM mixture and/or RM formulation do not contain a compound with a fluorinated C atom. The RM mixtures and RM formulations according to this preferred embodiment do thus enable a reduction of perfluorocarbons.

The RM mixture and/or RM formulation as described above and below, which do not contain a PFAS, more preferably do not contain a perfluorocarbon compound, very preferably do not contain compound with a polyfluorinated C atom, and most preferably do not contain a compound with a fluorinated C atom, are another object of the invention.

In another preferred embodiment the RM mixure or RM formulation comprises one or more specific antioxidant additives, preferably selected from the Irganox® series, e.g. the commercially available antioxidants Irganox®1076 and Irganox®1010, from Ciba, Switzerland.

In another preferred embodiment, the RM mixture or RM formulation comprises a combination of one or more, more preferably of two or more photoinitiators, for example, selected from the commercially available Irgacure® or Darocure® (Ciba AG) series, in particular, Irgacure 127, Irgacure 184, Irgacure 369, Irgacure 651, Irgacure 817, Irgacure 907, Irgacure 1300, Irgacure, Irgacure 2022, Irgacure 2100, Irgacure 2959, or Darcure TPO, further selected from the commercially available OXE02 (Ciba AG), NCI 930, N1919T (Adeka), SPI-03 or SPI-04 (Samyang), TR-PBG 304 or TR-PGB 345 (Tronly).

The photoinitiator is preferably selected such that it has an absorption maximum which is different from, very preferably at least 15 nm higher or lower than, the absorption maximum of the chiral photoisomerisable compound.

The concentration of the polymerisation initiator(s) as a whole in the RM mixure or RM formulation is preferably from 0.1 to 6%, very preferably from 0.3 to 5%, more preferably from 0.7 to 4%.

In another preferred embodiment the RM mixure or RM formulation optionally comprises one or more additives selected from polymerisable non-mesogenic compounds (reactive thinners). The amount of these additives in the RM mixture or RM formulation is preferably from 0 to 30%, very preferably from 0 to 25%.

The reactive thinners used are not only substances which are referred to in the actual sense as reactive thinners, but also auxiliary compounds already mentioned above which contain one or more complementary reactive units, for example hydroxyl, thiol-, or amino groups, via which a reaction with the polymerisable units of the liquid-crystalline compounds can take place.

The substances which are usually capable of photopolymerisation include, for example, mono-, bi- and polyfunctional compounds containing at least one olefinic double bond. Examples thereof are vinyl esters of carboxylic acids, for example of lauric, myristic, palmitic and stearic acid, and of dicarboxylic acids, for example of succinic acid, adipic acid, allyl and vinyl ethers and methacrylic and acrylic esters of monofunctional alcohols, for example of lauryl, myristyl, palmityl and stearyl alcohol, and diallyl and divinyl ethers of bifunctional alcohols, for example ethylene glycol and 1,4-butanediol.

Also suitable are, for example, methacrylic and acrylic esters of polyfunctional alcohols, in particular those which contain no further functional groups, or at most ether groups, besides the hydroxyl groups. Examples of such alcohols are bifunctional alcohols, such as ethylene glycol, propylene glycol and their more highly condensed representatives, for example diethylene glycol, triethylene glycol, dipropylene glycol, tripropylene glycol etc., butanediol, pentanediol, hexanediol, neopentyl glycol, alkoxylated phenolic compounds, such as ethoxylated and propoxylated bisphenols, cyclohexanedimethanol, trifunctional and polyfunctional alcohols, such as glycerol, trimethylolpropane, butanetriol, trimethylolethane, pentaerythritol, ditrimethylolpropane, dipentaerythritol, sorbitol, mannitol, and the corresponding alkoxylated, in particular ethoxylated and propoxylated alcohols.

Other suitable reactive thinners are polyester (meth)acrylates, which are the (meth)acrylic ester of polyesterols.

Examples of suitable polyesterols are those which can be prepared by esterification of polycarboxylic acids, preferably dicarboxylic acids, using polyols, preferably diols. The starting materials for such hydroxyl-containing polyesters are known to the person skilled in the art. Dicarboxylic acids which can be employed are succinic, glutaric acid, adipic acid, sebacic acid, o-phthalic acid and isomers and hydrogenation products thereof, and esterifiable and transesterifiable derivatives of said acids, for example anhydrides and dialkyl esters. Suitable polyols are the abovementioned alcohols, preferably ethyleneglycol, 1,2- and 1,3-propylene glycol, 1,4-butanediol, 1,6-hexanediol, neopentyl glycol, cyclohexanedimethanol and polyglycols of the ethylene glycol and propylene glycol type.

Suitable reactive thinners are furthermore 1,4-divinylbenzene, triallyl cyanurate, acrylic esters of tricyclodecenyl alcohol of the following formula

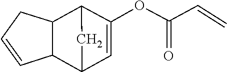

also known under the name dihydrodicyclopentadienyl acrylate, and the allyl esters of acrylic acid, methacrylic acid and cyanoacrylic acid.

Of the reactive thinners which are mentioned by way of example, those containing photopolymerisable groups are used in particular and in view of the abovementioned preferred compositions.

This group includes, for example, dihydric and polyhydric alcohols, for example ethylene glycol, propylene glycol and more highly condensed representatives thereof, for example diethylene glycol, triethylene glycol, dipropylene glycol, tripropylene glycol etc., butanediol, pentanediol, hexanediol, neopentyl glycol, cyclohexanedimethanol, glycerol, trimethylolpropane, butanetriol, trimethylolethane, pentaerythritol, ditrimethylolpropane, dipentaerythritol, sorbitol, mannitol and the corresponding alkoxylated, in particular ethoxylated and propoxylated alcohols.

The group furthermore also includes, for example, alkoxylated phenolic compounds, for example ethoxylated and propoxylated bisphenols.

These reactive thinners may furthermore be, for example, epoxide or urethane (meth)acrylates.

Epoxide (meth)acrylates are, for example, those as obtainable by the reaction, known to the person skilled in the art, of epoxidized olefins or poly- or diglycidyl ether, such as bisphenol A diglycidyl ether, with (meth)acrylic acid.

Urethane (meth)acrylates are, in particular, the products of a reaction, likewise known to the person skilled in the art, of hydroxylalkyl (meth)acrylates with poly- or diisocyanates.

Such epoxide and urethane (meth)acrylates are included amongst the compounds listed above as “mixed forms”.

If reactive thinners are used, their amount and properties must be matched to the respective conditions in such a way that, on the one hand, a satisfactory desired effect, for example the desired colour of the composition according to the invention, is achieved, but, on the other hand, the phase behaviour of the liquid-crystalline composition is not excessively impaired. The low-crosslinking (high-crosslinking) liquid-crystalline compositions can be prepared, for example, using corresponding reactive thinners which have a relatively low (high) number of reactive units per molecule.

The group of diluents include, for example:
C1-C4-alcohols, for example methanol, ethanol, n-propanol, isopropanol, butanol, isobutanol, sec-butanol and, in particular, the C5-C12-alcohols n-pentanol, n-hexanol, n-heptanol, n-octanol, n-nonanol, n-decanol, n-undecanol and n-dodecanol, and isomers thereof, glycols, for example 1,2-ethylene glycol, 1,2- and 1,3-propylene glycol, 1,2-, 2,3- and 1,4-butylene glycol, di- and triethylene glycol and di- and tripropylene glycol, ethers, for example methyl tert-butyl ether, 1,2-ethylene glycol mono- and dimethyl ether, 1,2-ethylene glycol mono- and -diethylether, 3-methoxypropanol, 3-isopropoxypropanol, tetrahydrofuran and dioxane, ketones, for example acetone, methyl ethyl ketone, methyl isobutyl ketone and diacetone alcohol (4-hydroxy-4-methyl-2-pentanone), C1-C5-alkyl esters, for example methyl acetate, ethyl acetate, propyl acetate, butyl acetate and amyl acetate, aliphatic and aromatic hydrocarbons, for example pentane, hexane, heptane, octane, isooctane, petroleum ether, toluene, xylene, ethylbenzene, tetralin, decalin, dimethylnaphthalene, white spirit, Shellsol® and Solvesso® mineral oils, for example gasoline, kerosine, diesel oil and heating oil, but also natural oils, for example olive oil, soya oil, rapeseed oil, linseed oil and sunflower oil.

It is of course also possible to use mixtures of these diluents in the compositions according to the invention.

So long as there is at least partial miscibility, these diluents can also be mixed with water. Examples of suitable diluents here are C1-C4-alcohols, for example methanol, ethanol, n-propanol, isopropanol, butanol, isobutanol and sec-butanol, glycols, for example 1,2-ethylene glycol, 1,2- and 1,3-propylene glycol, 1,2-, 2,3- and 1,4-butylene glycol, di- and triethylene glycol, and di- and tripropylene glycol, ethers, for example tetrahydrofuran and dioxane, ketones, for example acetone, methyl ethyl ketone and diacetone alcohol (4-hydroxy-4-methyl-2-pentanone), and C1-C4-alkyl esters, for example methyl, ethyl, propyl and butyl acetate.

The diluents are optionally employed in a proportion of from about 0 to 10.0% by weight, preferably from about 0 to 5.0% by weight, based on the total weight of the RM formulation.

In another preferred embodiment the RM mixture or RM formulation comprises one or more additives selected from the group consisting of antifoams and deaerators (c1)), lubricants and flow auxiliaries (c2)), thermally curing or radiation-curing auxiliaries (c3)), substrate wetting auxiliaries (c4)), wetting and dispersion auxiliaries (c5)), hydrophobicizing agents (c6)), adhesion promoters (c7)) and auxiliaries for promoting scratch resistance (c8)), wherein the additives of groups cannot always strictly be delimited from one another in their action.

For example, lubricants and flow auxiliaries often also act as antifoams and/or deaerators and/or as auxiliaries for improving scratch resistance. Radiation-curing auxiliaries can also act as lubricants and flow auxiliaries and/or deaerators and/or as substrate wetting auxiliaries. In individual cases, some of these auxiliaries can also fulfil the function of an adhesion promoter (c8)).

Corresponding to the above-said, a certain additive can therefore be classified in a number of the groups c1) to c8) described below.

The antifoams in group c1) include silicon-free and silicon-containing polymers. The silicon-containing polymers are, for example, unmodified or modified polydialkylsiloxanes or branched copolymers, comb or block copolymers comprising polydialkylsiloxane and polyether units, the latter being obtainable from ethylene oxide or propylene oxide.

The deaerators in group c1) include, for example, organic polymers, for example polyethers and polyacrylates, dialkylpolysiloxanes, in particular dimethylpolysiloxanes, organically modified polysiloxanes, for example arylalkyl-modified polysiloxanes, and fluorosilicones.

The action of the antifoams is essentially based on preventing foam formation or destroying foam that has already formed. Antifoams essentially work by promoting coalescence of finely divided gas or air bubbles to give larger bubbles in the medium to be deaerated, for example the compositions according to the invention, and thus accelerate escape of the gas (of the air). Since antifoams can frequently also be employed as deaerators and vice versa, these additives have been included together under group c1).

Such auxiliaries are, for example, commercially available from Tego as TEGO® Foamex 800, TEGO® Foamex 805, TEGO® Foamex 810, TEGO® Foamex 815, TEGO® Foamex 825, TEGO® Foamex 835, TEGO® Foamex 840, TEGO® Foamex 842, TEGO® Foamex 1435, TEGO® Foamex 1488, TEGO® Foamex 1495, TEGO® Foamex 3062, TEGO® Foamex 7447, TEGO® Foamex 8020, Tego® Foamex N, TEGO® Foamex K 3, TEGO® Antifoam 2-18, TEGO® Antifoam 2-18, TEGO® Antifoam 2-57, TEGO® Antifoam 2-80, TEGO® Antifoam 2-82, TEGO® Antifoam 2-89, TEGO® Antifoam 2-92, TEGO® Antifoam 14, TEGO® Antifoam 28, TEGO® Antifoam 81, TEGO® Antifoam D 90, TEGO® Antifoam 93, TEGO® Antifoam 200, TEGO® Antifoam 201, TEGO® Antifoam 202, TEGO® Antifoam 793, TEGO® Antifoam 1488, TEGO® Antifoam 3062, TEGOPREN® 5803, TEGOPREN® 5852, TEGOPREN® 5863, TEGOPREN® 7008, TEGO® Antifoam 1-60, TEGO® Antifoam 1-62, TEGO® Antifoam 1-85, TEGO® Antifoam 2-67, TEGO® Antifoam WM 20, TEGO® Antifoam 50, TEGO® Antifoam 105, TEGO® Antifoam 730, TEGO® Antifoam MR 1015, TEGO® Antifoam MR 1016, TEGO® Antifoam 1435, TEGO® Antifoam N, TEGO® Antifoam KS 6, TEGO® Antifoam KS 10, TEGO® Antifoam KS 53, TEGO® Antifoam KS 95, TEGO® Antifoam KS 100, TEGO® Antifoam KE 600, TEGO® Antifoam KS 911, TEGO® Antifoam MR 1000, TEGO® Antifoam KS 1100, Tego® Airex 900, Tego® Airex 910, Tego® Airex 931, Tego® Airex 935, Tego® Airex 936, Tego® Airex 960, Tego® Airex 970, Tego® Airex 980, and Tego® Airex 985, and from BYK as BYK®-011, BYK®-019, BYK®-020, BYK®-021, BYK®-022, BYK®-023, BYK®-024, BYK®-025, BYK®-027, BYK®-031, BYK®-032, BYK®-033, BYK®-034, BYK®-035, BYK®-036, BYK®-037, BYK®-045, BYK®-051, BYK®-052, BYK®-053, BYK®-055, BYK®-057, BYK®-065, BYK®-066, BYK®-070, BYK®-080, BYK®-088, BYK®-141 and BYK®-A 530.

The auxiliaries in group c1) are optionally employed in a proportion of from about 0 to 3.0% by weight, preferably from about 0 to 2.0% by weight, based on the total weight of the RM mixture or RM formulation.

In group c2), the lubricants and flow auxiliaries typically include silicon-free, but also silicon-containing polymers, for example polyacrylates or modifiers, low-molecular-weight polydialkylsiloxanes. The modification consists in some of the alkyl groups having been replaced by a wide variety of organic radicals. These organic radicals are, for example, polyethers, polyesters or even long-chain alkyl radicals, the former being used the most frequently.

The polyether radicals in the correspondingly modified polysiloxanes are usually built up from ethylene oxide and/or propylene oxide units. Generally, the higher the proportion of these alkylene oxide units in the modified polysiloxane, the more hydrophilic is the resultant product.

Such auxiliaries are, for example, commercially available from Tego as TEGO® Glide 100, TEGO® Glide ZG 400, TEGO® Glide 406, TEGO® Glide 410, TEGO® Glide 411, TEGO® Glide 415, TEGO® Glide 420, TEGO® Glide 435, TEGO® Glide 440, TEGO® Glide 450, TEGO® Glide A 115, TEGO® Glide B 1484 (can also be used as antifoam and deaerator), TEGO® Flow ATF, TEGO® Flow 300, TEGO® Flow 460, TEGO® Flow 425 and TEGO® Flow ZFS 460. Suitable radiation-curable lubricants and flow auxiliaries, which can also be used to improve the scratch resistance, are the products TEGO® Rad 2100, TEGO® Rad 2200, TEGO® Rad 2500, TEGO® Rad 2600 and TEGO® Rad 2700, which are likewise obtainable from TEGO.

Such-auxiliaries are available, for example, from BYK as BYK®-300 BYK®-306, BYK®-307, BYK®-310, BYK®-320, BYK®-333, BYK®-341, Byk® 354, Byk®361, Byk®361 N, BYK®388.

The auxiliaries in group c2) are optionally employed in a proportion of from about 0 to 3.0% by weight, preferably from about 0 to 2.0% by weight, based on the total weight of the RM mixture or RM formulation.

In group c3), the radiation-curing auxiliaries include, in particular, polysiloxanes having terminal double bonds which are, for example, a constituent of an acrylate group. Such auxiliaries can be crosslinked by actinic or, for example, electron radiation. These auxiliaries generally combine a number of properties together. In the uncrosslinked state, they can act as antifoams, deaerators, lubricants and flow auxiliaries and/or substrate wetting auxiliaries, while, in the crosslinked state, they increase, in particular, the scratch resistance, for example of coatings or films which can be produced using the compositions according to the invention. The improvement in the gloss properties, for example of precisely those coatings or films, is regarded essentially as a consequence of the action of these auxiliaries as antifoams, deaerators and/or lubricants and flow auxiliaries (in the uncrosslinked state).

Examples of suitable radiation-curing auxiliaries are the products TEGO® Rad 2100, TEGO® Rad 2200, TEGO® Rad 2500, TEGO® Rad 2600 and TEGO® Rad 2700 available from TEGO and the product BYK®-371 available from BYK.

Thermally curing auxiliaries in group c3) contain, for example, primary OH groups which are able to react with isocyanate groups, for example of the binder.

Examples of thermally curing auxiliaries which can be used are the products BYK®-370, BYK®-373 and BYK®-375 available from BYK.

The auxiliaries in group c3) are optionally employed in a proportion of from about 0 to 5.0% by weight, preferably from about 0 to 3.0% by weight, based on the total weight of the RM mixture or RM formulation.

The substrate wetting auxiliaries in group c4) serve, in particular, to increase the wettability of the substrate to be printed or coated, for example, by printing inks or coating compositions, for example compositions according to the invention. The generally attendant improvement in the lubricant and flow behaviour of such printing inks or coating compositions has an effect on the appearance of the finished (for example crosslinked) print or coating.

A wide variety of such auxiliaries are commercially available, for example from Tego as TEGO® Wet KL 245, TEGO® Wet 250, TEGO® Wet 260 and TEGO® Wet ZFS 453 and from BYK as BYK®-306, BYK®-307, BYK®-310, BYK®-333, BYK®-344, BYK®-345, BYK®-346 and Byk®-348.

The auxiliaries in group c4) are optionally employed in a proportion of from about 0 to 3.0% by weight, preferably from about 0 to 1.5% by weight, based on the total weight of the liquid-crystalline composition.

The wetting and dispersion auxiliaries in group c5) serve, in particular, to prevent the flooding and floating and the sedimentation of pigments and are therefore, if necessary, suitable in particular in pigmented compositions according to the invention.

These auxiliaries stabilize pigment dispersions essentially through electrostatic repulsion and/or steric hindrance of the pigment particles containing these additives, where, in the latter case, the interaction of the auxiliary with the ambient medium (for example binder) plays a major role.

Since the use of such wetting and dispersion auxiliaries is common practice, for example in the technical area of printing inks and paints, the selection of a suitable auxiliary of this type generally does not present the person skilled in the art with any difficulties, if they are used.

Such wetting and dispersion auxiliaries are commercially available, for example from Tego, as TEGO® Dispers 610, TEGO® Dispers 610 S, TEGO® Dispers 630, TEGO® Dispers 700, TEGO® Dispers 705, TEGO® Dispers 710, TEGO® Dispers 720 W, TEGO® Dispers 725 W, TEGO® Dispers 730 W, TEGO® Dispers 735 W and TEGO® Dispers 740 W and from BYK as Disperbyk®, Disperbyk®-107, Disperbyk®-108, Disperbyk®-110, Disperbyk®-111, Disperbyk®-115, Disperbyk®-130, Disperbyk®-160, Disperbyk®-161, Disperbyk®-162, Disperbyk®-163, Disperbyk®-164, Disperbyk®-165, Disperbyk®-166, Disperbyk®-167, Disperbyk®-170, Disperbyk®-174, Disperbyk®-180, Disperbyk®-181, Disperbyk®-182, Disperbyk®-183, Disperbyk®-184, Disperbyk®-185, Disperbyk®-190, Anti-Terra®-U, Anti-Terra®-U 80, Anti-Terra®-P, Anti-Terra®-203, Anti-Terra®-204, Anti-Terra®-206, BYK®-151, BYK®-154, BYK®-155, BYK®—P 104 S, BYK®—P 105, Lactimon®, Lactimon®-WS and Bykumen®.

The amount of the auxiliaries in group c5) used on the mean molecular weight of the auxiliary. In any case, a preliminary experiment is therefore advisable, but this can be accomplished simply by the person skilled in the art.

Another preferred group of auxiliaries, which can be allocated to group c2), c4) or c5), includes wetting-, flow- and leveling agents, in particular based on non-ionic fluorosurfactants, which are commercially available from Synthomer under the Polyfox™ series, for example Polyfox™PF-656.

The hydrophobicizing agents in group c6) can be used to give water-repellent properties to prints or coatings produced, for example, using compositions according to the invention. This prevents or at least greatly suppresses swelling due to water absorption and thus a change in, for example, the optical properties of such prints or coatings. In addition, when the composition is used, for example, as a printing ink in offset printing, water absorption can thereby be prevented or at least greatly reduced.

Such hydrophobicizing agents are commercially available, for example, from Tego as Tego® Phobe WF, Tego® Phobe 1000, Tego® Phobe 1000 S, Tego® Phobe 1010, Tego® Phobe 1030, Tego® Phobe 1010, Tego® Phobe 1010, Tego® Phobe 1030, Tego® Phobe 1040, Tego® Phobe 1050, Tego® Phobe 1200, Tego® Phobe 1300, Tego® Phobe 1310 and Tego® Phobe 1400.

The auxiliaries in group c6) are optionally employed in a proportion of from about 0 to 5.0% by weight, preferably from about 0 to 3.0% by weight, based on the total weight of the RM mixture or RM formulation.

Adhesion promoters from group c7) serve to improve the adhesion of two interfaces in contact. It is directly evident from this that essentially the only fraction of the adhesion promoter that is effective is that located at one or the other or at both interfaces. If, for example, it is desired to apply liquid or pasty printing inks, coating compositions or paints to a solid substrate, this generally means that the adhesion promoter must be added directly to the latter or the substrate must be pre-treated with the adhesion promoters (also known as priming), i.e. this substrate is given modified chemical and/or physical surface properties.

If the substrate has previously been primed with a primer, this means that the interfaces in contact are that of the primer on the one hand and of the printing ink or coating composition or paint on the other hand. In this case, not only the adhesion properties between the substrate and the primer, but also between the substrate and the printing ink or coating composition or paint play a part in adhesion of the overall multilayer structure on the substrate.

Adhesion promoters in the broader sense which may be mentioned are also the substrate wetting auxiliaries already listed under group c4), but these generally do not have the same adhesion promotion capacity.

In view of the widely varying physical and chemical natures of substrates and of printing inks, coating compositions and paints intended, for example, for their printing or coating, the multiplicity of adhesion promoter systems is not surprising.

Adhesion promoters based on silanes are, for example, 3-aminopropyltrimethoxysilane, 3-aminopropyltriethoxysilane, 3-aminopropylmethyldiethoxysilane, N-aminoethyl-3-aminopropyltrimethoxysilane, N-aminoethyl-3-aminopropylmethyldimethoxysilane, N-methyl-3-aminopropyltrimethoxysilane, 3-ureidopropyltriethoxysilane, 3-methacryloyloxypropyltrimethoxysilane, 3-glycidyloxypropyltrimethoxysilane, 3-mercaptopropyltrimethoxysilane, 3-chloropropyltrimethoxysilane and vinyltrimethoxysilane.

These and other silanes are commercially available from Hüls, for example under the tradename DYNASILAN®.

Corresponding technical information from the manufacturers of such additives should generally be used or the person skilled in the art can obtain this information in a simple manner through corresponding preliminary experiments.

However, if these additives are to be added as auxiliaries from group c7) to the RM mixtures or RM formulations according to the invention, their proportion optionally corresponds to from about 0 to 5.0% by weight, based on the total weight of the RM mixture or RM formulation. These concentration data serve merely as guidance, since the amount and identity of the additive are determined in each individual case by the nature of the substrate and of the printing/coating composition. Corresponding technical information is usually available from the manufacturers of such additives for this case or can be determined in a simple manner by the person skilled in the art through corresponding preliminary experiments.

The auxiliaries for improving the scratch resistance in group c8) include, for example, the abovementioned products TEGO® Rad 2100, TEGO® Rad 2200, TEGO® Rad 2500, TEGO® Rad 2600 and TEGO® Rad 2700, which are available from Tego.

For these auxiliaries, the amount data given for group c3) are likewise suitable, i.e. these additives are optionally employed in a proportion of from about 0 to 5.0% by weight, preferably from about 0 to 3.0% by weight, based on the total weight of the liquid-crystalline composition.

Examples which may be mentioned of light, heat and/or oxidation stabilizers are the following:
alkylated monophenols, such as 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which have a linear or branched side chain, for example 2,6-dinonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures of these compounds, alkylthiomethylphenols, such as 2,4-dioctylthiomethyl-6-tert-butylphenol, 2,4-dioctylthiomethyl-6-methylphenol, 2,4-dioctylthiomethyl-6-ethylphenol and 2,6-didodecylthiomethyl-4-nonylphenol,
Hydroquinones and alkylated hydroquinones, such as 2,6-di-tert-butyl-4-methoxyphenol, 2,5-di-tert-butylhydroquinone, 2,5-di-tert-amylhydrocrainone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate and bis(3,5-di-tert-butyl-4-hydroxyphenyl)adipate,
Tocopherols, such as α-tocopherol, β-tocopherol, γ-tocopherol, δ-tocopherol and mixtures of these compounds, and tocopherol derivatives, such as tocopheryl acetate, succinate, nicotinate and polyoxyethylenesuccinate (“tocofersolate”),
hydroxylated diphenyl thioethers, such as 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol) and 4,4′-bis(2,6-dimethyl-4-hydroxyphenyl)disulfide,
Alkylidenebisphenols, such as 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecyl-mercaptobutane, ethylene glycol bis[3,3-bis(3′-tert-butyl-4′-hydroxyphenyl)butyrate], bis(3-tert-butyl-4-hydroxy-5-methylphenyl)dicyclopentadiene, bis[2-(3′-tert-butyl-2′-hydroxy-5′-methylbenzyl)-6-tert-butyl-4-methylphenyl]terephthalate, 1,1-bis(3,5-dimethyl-2-hydroxyphenyl)butane, 2,2-bis(3,5-di-tert-butyl-4-hydroxyphenyl)propane, 2,2-bis(5-tert-butyl-4-hydroxy-2-methylphenyl)-4-n-dodecyl-mercaptobutane and 1,1,5,5-tetrakis(5-tert-butyl-4-hydroxy-2-methylphenyl)pentane,
O—, N- and S-benzyl compounds, such as 3,5,3′,5′-tetra-tert-butyl-4,4′-dihydroxydibenzyl ether, octadecyl 4-hydroxy-3,5-dimethylbenzylmercaptoacetate, tridecyl 4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide and isooctyl-3,5-di-tert-butyl-4-hydroxybenzylmercaptoacetate,
aromatic hydroxybenzyl compounds, such as 1,3,5-tris(3,5-di-tert-butyl-4-hydroxybenzyl)-2,4,6-trimethyl-benzene, 1,4-bis(3,5-di-tert-butyl-4-hydroxybenzyl)-2,3,5,6-tetramethyl-benzene and 2,4,6-tris(3,5-di-tert-butyl-4-hydroxybenzyl)phenol,
Triazine compounds, such as 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris-(3,5-di-tert-butyl-4-hydroxyphenylpropionyl)hexahydro-1,3,5-triazine, 1,3,5-tris(3,5-dicyclohexyl-4-hydroxybenzyl)isocyanurate and 1,3,5-tris(2-hydroxyethyl)isocyanurate,
Benzylphosphonates, such as dimethyl 2,5-di-tert-butyl-4-hydroxybenzylphosphonate, diethyl 3,5-di-tert-butyl-4-hydroxybenzylphosphonate, dioctadecyl 3,5-di-tert-butyl-4-hydroxybenzylphosphonate and dioctadecyl 5-tert-butyl-4-hydroxy-3-methylbenzylphosphonate,
Acylaminophenols, such as 4-hydroxylauroylanilide, 4-hydroxystearoylanilide and octyl N-(3,5-di-tert-butyl-4-hydroxyphenyl)carbamate,
Propionic and acetic esters, for example of monohydric or polyhydric alcohols, such as methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxalamide, 3-thiaundecanol, 3-thiapentadecanol, trimethylhexanediol, trimethylolpropane and 4-hydroxymethyl-1-phospha-2,6,7-trioxabicyclo[2.2.2]-octane,
Propionamides based on amine derivatives, such as N,N′-bis(3,5-di-tert-butyl-4-hydroxyphenylpropionyl)hexamethylenediamine, N,N′-bis(3,5-di-tert-butyl-4-hydroxyphenylpropionyl)trimethylenediamine and N,N′-bis(3,5-di-tert-butyl-4-hydroxyphenylpropionyl)hydrazine,
Ascorbic acid (Vitamin C) and ascorbic acid derivatives, such as ascorbyl palmitate, laurate and stearate, and ascorbyl sulfate and phosphate,
Antioxidants based on amine compounds, such as N,N′-diisopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octyl-substituted diphenylamine, such as p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis[4-methoxyphenyl)amine, 2,6-di-tert-butyl-4-dimethylaminomethylphenol, 2,4-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octyl-substituted N-phenyl-1-naphthylamine, a mixture of mono- and dialkylated tert-butyl/tert-octyldiphenylamine, a mixture of mono- and dialkylated nonyldiphenylamine, a mixture of mono- and dialkylated dodecyldiphenylamine, a mixture of mono- and dialkylated isopropyl/isohexyldiphenylamine, a mixture of mono- and dialkylated tert-butyldiphenylamine, 2,3-dihydro-3,3-dimethyl-4H-1,4-benzothiazine, phenothiazine, a mixture of mono- and dialkylated tert-butyl/tert-octylphenothiazine, a mixture of mono- and dialkylated tert-octylphenothiazine, N-allylphenothiazine, N,N,N′,N′-tetraphenyl-1,4-diaminobut-2-ene, N,N-bis(2,2,6,6-tetramethylpiperidin-4-yl)hexamethylenediamine, bis(2,2,6,6-tetramethylpiperidin-4-yl)sebacate, 2,2,6,6-tetramethylpiperidin-4-one and 2,2,6,6-tetramethylpiperidin-4-ol,
Phosphines, Phosphites and phosphonites, such as triphenylphosnine triphenylphosphite, diphenyl alkyl phosphite, phenyl dialkyl phosphite, tris(nonylphenyl)phosphite, trilauryl phosphite, trioctadecyl phosphite, distearyl pentaerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,6-di-tert-butyl-4-methylphenyl)pentaerythritol diphosphite, diisodecyloxy pentaerythritol diphosphite, bis(2,4-di-tert-butyl-6-methylphenyl)pentaerythritol diphosphite, bis(2,4,6-tris(tert-butylphenyl))pentaerythritol diphosphite, tristearyl sorbitol triphosphite, tetrakis(2,4-di-tert-butylphenyl)4,4′-biphenylenediphosphonite, 6-isooctyloxy-2,4,8,10-tetra-tert-butyl-12H-dibenz[d,g]-1,3,2-dioxaphosphocine, 6-fluoro-2,4,8,10-tetra-tert-butyl-12-methyl-dibenz[d,g]-1,3,2-dioxaphosphocine, bis(2,4-di-tert-butyl-6-methylphenyl)methyl phosphite and bis(2,4-di-tert-butyl-6-methylphenyl)ethyl phosphite,
2-(2′-Hydroxyphenyl)benzotriazoles, such as 2-(2′-hydroxy-5′-methylphenyl)benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl)-5-chlorobenzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3,5′-bis-(α,α-dimethylbenzyl)-2′-hydroxyphenyl)benzotriazole, a mixture of 2-(3′-tert-butyl-2′-hydroxy-5′-(2-octyloxycarbonylethyl)phenyl)-5-chlorobenzotriazole, 2-(3′-tert-butyl-5′-[2-(2-ethylhexyloxy)carbonylethyl]-2′-hydroxy phenyl)-5-chlorobenzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxy phenyl)benzotriazole, 2-(3′-dodecyl-2′-hydroxy-5′-methylphenyl)benzotriazole and 2-(3′-tert-butyl-2′-hydroxy-5′-(2-isooctyloxycarbonylethyl)phenyl benzotriazole, 2,2′-methylenebis[4-(1,1,3,3-tetramethylbutyl)-6-benzotriazol-2-ylphenol]; the product of complete esterification of 2-[3′-tert-butyl-5′-(2-methoxycarbonylethyl)-2′-hydroxyphenyl]-2H-benzotriazole with polyethylene glycol 300;
sulfur-containing peroxide scavengers and sulfur-containing antioxidants, such as esters of 3,3′-thiodipropionic acid, for example the lauryl, stearyl, myristyl and tridecyl esters, mercaptobenzimidazole and the zinc salt of 2-mercaptobenzimidazole, dibutylzinc dithiocarbamates, dioctadecyl disulfide and pentaerythritol tetrakis(β-dodecylmercapto)propionate,
2-hydroxybenzophenones, such as the 4-hydroxy, 4-methoxy, 4-octyloxy, 4-decycloxy, 4-dodecyloxy, 4-benzyloxy, 4,2′,4′-trihydroxy and 2′-hydroxy-4,4′-dimethoxy derivatives,
Esters of unsubstituted and substituted benzoic acids, such as 4-tert-butylphenyl salicylate, phenyl salicylate, octylphenyl salicylate, dibenzoylresorcinol, bis(4-tert-butylbenzoyl)resorcinol, benzoylresorcinol, 2,4-di-tert-butylphenyl 3,5-di-tert-butyl-4-hydroxybenzoate, hexadecyl-3,5-di-tert-butyl-4-hydroxybenzoate, octadecyl-3,5-di-tert-butyl-4-hydroxybenzoate and 2-methyl-4,6-di-tert-butylphenyl-3,5-di-tert-butyl-4-hydroxybenzoate, Acrylates, such as ethyl α-cyano-β,β-diphenylacrylate, isooctyl α-cyano-β,β-diphenylacrylate, methyl α-methoxycarbonylcinnamate, methyl α-cyano-β-methyl-p-methoxycinnamate, butyl-α-cyano-β-methyl-p-methoxycinnamate and methyl-α-methoxycarbonyl-p-methoxycinnamate, sterically hindered amines, such as bis(2,2,6,6-tetramethylpiperidin-4-yl)sebacate, bis(2,2,6,6-tetramethylpiperidin-4-yl)succinate, bis(1,2,2,6,6-pentamethylpiperidin-4-yl)sebacate, bis(1-octyloxy-2,2,6,6-tetramethylpiperidin-4-yl)sebacate, bis(1,2,2,6,6-pentamethylpiperidin-4-yl)-n-butyl-3,5-di-tert-butyl-4-hydroxybenzylmalonate, the condensation product of 1-(2-hydroxyethyl)-2,2,6,6-tetramethyl-4-hydroxypiperidine and succinic acid, the condensation product of N,N′-bis(2,2,6,6-tetramethylpiperidin-4-yl)hexamethylenediamine and 4-tert-octylamino-2,6-dichloro-1,3,5-triazine, tris(2,2,6,6-tetramethylpiperidin-4-yl)nitrilotriacetate, tetrakis(2,2,6,6-tetramethylpiperidin-4-yl)1,2,3,4-butanetetracarboxylate, 1,1′-(1,2-ethylene)bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidin-4-yl)2-n-butyl-2-(2-hydroxy-3,5-di-tert-butylbenzyl)malonate, 3-n-octyl-7,7,9,9-tetramethyl-1,3,8-triazaspiro[4.5]decane-2,4-dione, bis(1-octyloxy-2,2,6,6-tetramethylpiperidin-4-yl)sebacate, bis(1-octyloxy-2,2,6,6-tetramethylpiperidin-4-yl)succinate, the condensation product of N,N′-bis(2,2,6,6-tetramethylpiperidin-4-yl)hexamethylenediamine and 4-morpholino-2,6-dichloro-1,3,5-triazine, the condensation product of 2-chloro-4,6-bis(4-n-butylamino-2,2,6,6-tetramethylpiperidin-4-yl)-1,3,5-triazine and 1,2-bis(3-aminopropylamino)ethane, the condensation product of 2-chloro-4,6-di(4-n-butylamino-1,2,2,6,6-pentamethylpiperidin-4-yl)-1,3,5-triazine and 1,2-bis(3-aminopropylamino)ethane, 8-acetyl-3-dodecyl-7,7,9,9-tetramethyl-1,3,8-triazaspiro[4.5]-decane-2,4-dione, 3-dodecyl-1-(2,2,6,6-tetramethylpiperidin-4-yl)pyrrolidine-2,5-dione, 3-dodecyl-1-(1,2,2,6,6-pentamethylpiperidin-4-yl)pyrrolidine-2,5-dione, a mixture of 4-hexadecyloxy- and 4-stearyloxy-2,2,6,6-tetramethylpiperidine, the condensation product of N,N′-bis(2,2,6,6-tetramethylpiperidin-4-yl)hexamethylenediamine and 4-cyclohexylamino-2,6-dichloro-1,3,5-triazine, the condensation product of 1,2-bis(3-aminopropylamino)ethane and 2,4,6-trichloro-1,3,5-triazine, 4-butylamino-2,2,6,6-tetramethylpiperidine, N-(2,2,6,6-tetramethylpiperidin-4-yl)-n-dodecylsuccinimide, N-(1,2,2,6,6-pentamethylpiperidin-4-yl)-n-dodecylsuccinimide, 2-undecyl-7,7,9,9-tetramethyl-1-oxa-3,8-diaza-4-oxo-spiro[4.5]-decane, the condensation product of 7,7,9,9-tetramethyl-2-cycloundecyl-1-oxa-3,8-diaza-4-oxospiro-[4.5]decane and epichlorohydrin, the condensation products of 4-amino-2,2,6,6-tetramethylpiperidine with tetramethylolacetylenediureas and poly(methoxypropyl-3-oxy)-[4(2,2,6,6-tetramethyl)piperidinyl]-siloxane,
Oxalamides, such as 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxalamide, 2-ethoxy-5-tert-butyl-2′-ethoxanilide and its mixture with 2-ethoxy-2′-ethyl-5,4′-di-tert-butoxanilide, and mixtures of ortho-, para-methoxy-disubstituted oxanilides and mixtures of ortho- and para-ethoxy-disubstituted oxanilides, and
2-(2-hydroxyphenyl)-1,3,5-triazines, such as 2,4,6-tris-(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropoxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis-(2,4-dimethylphenyl)-1,3,5-triazine, 2-(2-hydroxy-4-hexyloxyphenyl)-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine and 2-(2-hydroxyphenyl)-4-(4-methoxyphenyl)-6-phenyl-1,3,5-triazine.

In a preferred embodiment the RM mixture or RM formulation is dissolved in a suitable solvent, which are preferably selected from organic solvents.

The solvents are preferably selected from ketones such as acetone, methyl ethyl ketone, methyl propyl ketone, methyl isobutyl ketone or cyclohexanone; acetates such as methyl, ethyl or butyl acetate or methyl acetoacetate; alcohols such as methanol, ethanol or isopropyl alcohol; aromatic solvents such as toluene or xylene; alicyclic hydrocarbons such as cyclopentane or cyclohexane; halogenated hydrocarbons such as di- or trichloromethane; glycols or their esters such as PGMEA (propyl glycol monomethyl ether acetate), Q-butyrolactone. It is also possible to use binary, ternary or higher mixtures of the above solvents. In particular, for multilayer applications, methyl iso butyl ketone is the preferred utilized solvent

In case the RM formulation contains one or more solvents, the total concentration of all solids, including the RMs, in the solvent(s) is preferably from 5 to 60%, more preferably from 10 to 50%, in particular from 10 to 35%.

Preferably, the RM mixture or RM formulation comprises, in addition to one or more compounds or formula I one or more components selected from the group consisting of components a) to q), or any combination thereof:
a) one or more multi—or direactive polymerisable mesogenic compounds, preferably selected from compounds of formula DRM and corresponding subformulae, and/or
b) one or more monoreactive polymerisable mesogenic compounds, preferably selected from compounds of formula MRM and corresponding subformulae, and/or
c) one or more polymerisable chiral compounds, preferably selected from formulae CRM or its subformulae, and/or
d) one or more chiral isomerisable compounds, which can be polymeirsable or non-polymerisable, preferably selected from formula I*,
e) one or more non-polymerisable and non-isomerisable chiral compounds, preferably selected from formulae C—I, C-II and C—III, and/or
f) one or more photoinitiators, and/or
g) one or more antioxidative additives, and/or
h) one or more adhesion promotors, and/or
i) one or more surfactants, and/or
j) one or more mono-, di- or multireactive polymerisable non-mesogenic compounds, and/or
k) one or more dyes showing an absorption maximum at the wavelength used to initiate photo polymerisation, and/or
l) one or more chain transfer agents, and/or
m) one or more (UV) stabilizers, and/or
n) one or more lubricants and flow auxiliaries, and
o) one or more diluents, and/or
p) a non-polymerisable nematic component, and/or
q) one or more organic solvents.

More preferably, the RM mixture or RM formulation comprises:
1) one or more compounds of formula I, or its corresponding preferred subformulae, and
2) optionally one or more, preferably two or more, direactive polymerisable mesogenic compounds, preferably selected from the compounds of formula DRMa-1, and/or
3) optionally one or more, preferably two or more, monoreactive polymerisable mesogenic compounds, preferably selected from compounds of formulae MRM-1, and/or MRM-4, and/or MRM-6, and/or MRM-7, and/or
4) optionally one or more polymerisable chiral compounds, preferably selected from formulae CRM or its subformulae, and/or
5) optionally one or more chiral isomerisable compounds, preferably selected from formula I*, more preferably from formula I*A, or their corresponding preferred subformulae, and/or
6) optionally one or more non-polymerisable and non-isomerisable chiral compounds, preferably selected from formulae C—I, C-II and C—III, and/or
7) optionally one or more antioxidative additives, and/or
8) optionally one or more photoinitiators, and/or
9) optionally one or more organic solvents,
   or any combination of the aforementioned components 1) to 9), provided that at least component 1) is present.

The RM mixture and RM formulation can be prepared in a manner conventional per se, for example by mixing one or more of the above-mentioned chiral isomerisable compounds with one or more RMs as defined above, and optionally with further additives.

The invention further relates to a process of preparing a polymer film by polymerising a compound of formula I or an RM mixture according to the present invention, preferably wherein the polymerisable compounds are aligned into uniform orientation, and preferably at a temperature where the polymerisable compounds or the RM mixture exhibit a liquid crystal phase, preferably a nematic or a cholesteric (chiral nematic) phase.

A first preferred embodiment of the invention relates to a process of preparing a polymer film by polymerising a compound of formula I or an RM mixture according to the present invention, preferably wherein the polymerisable compounds are aligned into comprising, preferably consisting of, the steps of
providing a layer of an RM mixture or RM formulation as described above and below onto a substrate, which is optionally provided with an alignment layer capable of inducing a planar alignment to the adjacent layer of the RM mixture,
optionally removing any solvents, if present,
optionally annealing the layer of the RM mixture (i.e., without solvent), preferably at a temperature where it is in the nematic or chiral nematic phase,
irradiating the RM layer with actinic radiation, preferably with UV radiation, causing polymerisation of the polymerisable components and formation of a polymer film,
optionally removing the polymer film from the substrate.

A second preferred embodiment of the invention relates to a process of preparing a polymer film from an RM mixture according to the present invention which contains a chiral compound that is isomerisable and preferably also polymerisable. The process of this second preferred embodiment comprises two irradiations steps, and more precisely comprises, preferably consists of, the steps of
providing a layer of an RM mixture or RM formulation as described above and below, which contains a chiral compound that is isomerisable and preferably also polymerisable, onto a substrate, which is optionally provided with an alignment layer capable of inducing a planar alignment to the adjacent layer of the RM mixture,
optionally removing any solvents, if present,
optionally annealing the layer of the RM mixture (i.e., without solvent), preferably at a temperature where it is in the chiral nematic phase,
a first step of irradiation of the RM layer with actinic radiation, preferably with UV radiation, in air (1^{st} UV step),
optionally annealing the layer of the RM mixture, preferably at a temperature where it is in the chiral nematic phase, and
a second step of irradiation of the RM layer with actinic radiation, preferably with UV radiation, in an inert gas atmosphere (2^{nd} UV step),
optionally removing the formed polymer film from the substrate.

More preferably the process of preparing a polymer film according to the second preferred embodiment of the present invention comprises the following steps:
providing a layer of an RM mixture or RM formulation as described above and below, or a solution thereof, onto a substrate, which is preferably equipped with an alignment layer inducing planar alignment layer, for example a rubbed polyimide layer or a photo alignment layer, for example by spin-coating or printing methods, and optionally removing any solvents present,
optionally annealing the layer of the RM mixture (i.e., without solvent), preferably at a temperature where it is in the chiral nematic phase,
exposing the layer of the RM mixture (i.e., without solvent) to UV light, which causes photoisomerisation of the chiral compound comprising the photoisomerisable group and provides the chiral structure with the biased helical pitch, preferably to unpolarised UV light, very preferably to unpolarised UVA light, for example with a dose of 40 to 500 mJ/cm², preferably in an air environment at ambient temperature (“1^{st} UV step”),
optionally annealing the layer of the RM mixture, preferably at a temperature where it is in the chiral nematic phase,
exposing the layer of the RM mixture to UV light, which causes photopolymerisation of the RMs, preferably to unpolarised UV light, very preferably to unpolarised UVA light, for example with a dose of 200 to 2000 mJ/cm²cm², preferably in an inert gas atmosphere, e.g. nitrogen and at ambient temperature (“2^{nd} UV step”),
optionally removing the polymer film from the substrate.

The invention further relates to a polymer film obtainable by a process as described above and below.

Preferably in the processes according to the present invention all irradiation or UV exposure steps are carried out at room temperature, and the layer of the polymerisable LC medium is not subjected to heat treatment during or between the irradiation or UV exposure steps.

In case of the two-step irradiation process according to the second preferred embodiment, the first irradiation or 1^{st} UV step causes photoisomerisation of the chiral compound comprising the photoisomerisable group and provides the chiral structure with the biased helical pitch. The second irradiation or 2^{nd} UV step causes photopolymerisation of the polymerisable mesogenic compounds and thereby fixes the chiral structure. This process can be advantageously used to obtain a chiral pitch gradient in the film thickness direction, wherein the chiral rotation angle increases or decreases incrementally through the film thickness.

This RM mixture or RM formulation can be coated or printed onto the substrate, for example by spin-coating, printing, or other known techniques, and the solvent is evaporated off before polymerisation. In most cases, it is suitable to heat the mixture in order to facilitate the evaporation of the solvent.

The RM mixture or RM formulation can be applied onto a substrate by conventional coating techniques like spin coating, bar coating or blade coating. It can also be applied to the substrate by conventional printing techniques which are known to the expert, like for example screen printing, offset printing, reel-to-reel printing, letter press printing, gravure printing, rotogravure printing, flexographic printing, intaglio printing, pad printing, heat-seal printing, ink-jet printing or printing by means of a stamp or printing plate.

Suitable substrate mediums and substrates are known to the expert and described in the literature, as for example conventional substrates used in the optical films industry, such as glass or plastic. Especially suitable and preferred substrates for polymerisation are polyester such as polyethyleneterephthalate (PET) or polyethylenenaphthalate (PEN), polyvinylalcohol (PVA), polycarbonate (PC), triacetylcellulose (TAC), cyclo-olefin polymers (COP), or commonly known color filter materials, preferably triacetylcellulose (TAC), cyclo-olefin polymers (COP), or commonly known colour filter materials.

In another preferred embodiment the substrate has a surface grating or surface pattern, preferably a diffraction grating, very preferably a PB grating. In another preferred embodiment the substrate is prepared from a photoalignment layer (PAL) which is patterned by laser interferometry to create a grating pattern with a defined pitch.

The Friedel-Creagh-Kmetz rule can be used to predict whether a mixture will adopt planar or homeotropic alignment, by comparing the surface energies of the RM layer (γ_{RM}) and the substrate (γₛ):

If γ_{RM}>γₛ the reactive mesogenic compounds will display homeotropic alignment, If γ_{RM}<γₛ the reactive mesogenic compounds will display homogeneous alignment.

Without wishing to be bound to a specific theory, it is believed that when the surface energy of a substrate is relatively low, the intermolecular forces between the reactive mesogens are stronger than the forces across the RM-substrate interface and consequently, reactive mesogens align perpendicular to the substrate (homeotropic alignment) in order to maximise the intermolecular forces. Accordingly, an additional alignment layer capable of inducing a planar alignment to the adjacent polymerisable LC medium is required.

When the surface tension of the substrate is greater than the surface tension of the RMs, the force across the interface dominates. The interface energy is minimised if the reactive mesogens align parallel with the substrate, so the long axis of the RM can interact with the substrate. One way planar alignment can be promoted is by coating the substrate with a polyimide layer, and then rubbing the alignment layer with a velvet cloth.

Other suitable planar alignment layers are known in the art, like for example rubbed polyimide or alignment layers prepared by photoalignment as described in U.S. Pat. Nos. 5,602,661, 5,389,698 or 6,717,644.

In general, reviews of alignment techniques are given for example by I. Sage in “Thermotropic Liquid Crystals”, edited by G. W. Gray, John Wiley & Sons, 1987, pages 75-77; and by T. Uchida and H. Seki in “Liquid Crystals—Applications and Uses Vol. 3”, edited by B. Bahadur, World Scientific Publishing, Singapore 1992, pages 1-63. A further review of alignment materials and techniques is given by J. Cognard, Mol. Cryst. Liq. Cryst. 78, Supplement 1 (1981), pages 1-77.

In a preferred embodiment, the process according to the invention contains a process step where the RM mixture or RM formulation is allowed to rest for a period of time in order to evenly redistribute the polymerisable LC medium on the substrate (herein referred to as “annealing”).

In a preferred embodiment, after providing the RM mixture or RM formulation onto the substrate, the layer stack is annealed for a time between 10 seconds and 1 hour, preferably between 20 seconds and 10 minutes and most preferably between 30 seconds and 2 minutes. The annealing is preferably performed at room temperature.

The RM mixture preferably consists of compounds that aling spontaneously when being deposited as a mixture onto the substrate. Therefore, preferably the RM mixture is not subjected to heat treatment to align the mesogenic or liquid-crystalline compounds before the UV exposure.

If necessary, the layer stack can be cooled down to room temperature after annealing at an elevated temperature. The cooling can be performed actively with the help of cooling aids or passively just by letting the layer stack rest for a given time.

In a preferred embodiment, in the 1^{st} UV step the RM mixture is exposed to actinic radiation as described for example in WO 01/20394, GB 2,315,072 or WO 98/04651.

Actinic radiation means irradiation with light, like UV light, IR light or visible light, irradiation with X-rays or gamma rays, or irradiation with high-energy particles, such as ions or electrons. Preferably, the 1^{st} UV step is carried out by photo irradiation, in particular with UV light, especially with UVA light.

As a source for actinic radiation, for example a single UV lamp or a set of UV lamps can be used. When using a high lamp power the curing time can be reduced. Another possible source for photo radiation is a laser, like e.g. a UV laser, an IR laser, or a visible laser.

The curing time is dependent, inter alia, on the reactivity of the polymerisable and photoreactive compounds, the thickness of the coated layer, and the power and selected wavelength of the UV lamp. The curing time is preferably ≤5 minutes, very preferably ≤3 minutes, most preferably ≤1 minute. For mass production, short curing times of ≤30 seconds are preferred.

A suitable UV radiation power in the 1^{st} UV step is preferably in the range from 5 to 300 mWcm⁻², more preferably in the range from 50 to 250 mWcm⁻² and most preferably in the range from 100 to 180 mWcm⁻².

In connection with the applied UV radiation and as a function of time, a suitable UV dose is preferably in the range from 20 to 1000 mJcm⁻², more preferably in the range from 30 to 800 mJcm⁻², very preferably in the range from 40 to 500 mJcm⁻², most preferably in the range from 40 to 200 mJcm⁻².

In case of the two-step irradiation process according to the second preferred embodiment, the first irradiation step or 1^{st} UV step for isomerising the chiral compound is preferably performed in air. Preferably the first irradiation step or 1^{st} UV step is preferably performed at room temperature.

Photopolymerisation of the RM mixture is preferably achieved by exposing it to actinic radiation. Actinic radiation means irradiation with light, like UV light, IR light or visible light, irradiation with X-rays or gamma rays, or irradiation with high-energy particles, such as ions or electrons. Preferably, polymerisation is carried out by photo irradiation, in particular with UV light. As a source for actinic radiation, for example a single UV lamp or a set of UV lamps can be used. When using a high lamp power the curing time can be reduced. Another possible source for photo radiation is a laser, like e.g. a UV laser, an IR laser, or a visible laser.

The curing time for the photopolymerisation is dependent, inter alia, on the reactivity of the polymerisable LC medium, the thickness of the coated layer, the type of polymerisation initiator and the power of the UV lamp. The curing time is preferably ≤5 minutes, very preferably ≤3 minutes, most preferably ≤1 minute. For mass production, short curing times of ≤30 seconds are preferred.

A suitable UV radiation power for the photopolymerisation is preferably in the range from 100 to 1000 mWcm⁻², more preferably in the range from 200 to 800 mWcm⁻² and most preferably in the range from 300 to 600 mWcm⁻².

In connection with the applied UV radiation and as a function of time, a suitable UV dose is preferably in the range from 25 to 16500 mJcm⁻², more preferably in the range from 50 to 7200 mJcm⁻², very preferably in the range from 100 to 3500 mJcm⁻² and most preferably in the range from 200 to 2000 mJcm⁻².

Photopolymerisation (or the second irradiation step or 2^{nd} UV step of the two-step process) is preferably performed under an inert gas atmosphere, preferably in a nitrogen atmosphere. Further preferably photopolymerisation (or the second irradiation step or 2^{nd} UV step in the two-step process) is preferably performed at room temperature.

The preferred thickness of a polymerised LC film according to the present invention is determined by the optical properties desired from the film or the final product.

For optical applications of the polymer film, it preferably has a thickness of from 0.1 to 10 μm, very preferably from 0.1 to 2 μm, in particular from 0.1 to 1 μm.

In a preferred embodiment, the polymer film according to the present invention shows planar alignment, i.e., the LC molecules are oriented parallel to the film plane and the helical axis is oriented substantially perpendicular to the film plane.

In another preferred embodiment the polymer film according to the present invention shows tilted alignment, i.e., the LC molecules are oriented at an angle to the film plane and the helical axis is oriented at an angle to the film plane, also referred as tilt angle. In a tilted film, the tilt angle between the helix axis and the axis normal to the film plane is from 5° to 45°, very preferably from 15° to 45°.

In another preferred embodiment, the tilt angle between the helix axis and the axis normal to the film plane is from 0 to 15°, very preferably from 0 to 5°.

Planar alignment can be induced for example by providing an alignment layer on the substrate, for example a polyimide alignment layer, as described above. Tilted alignment can be achieved for example by adding an alignment additive to the chiral RM mixture, or by using a substrate with a surface grating or pattern, e.g. a PB grating.

The optical retardation (δ(λ)) of a polymer film as a function of the wavelength of the incident beam (λ) is given by the following equation (7):

$$δ ( λ ) = ( 2 πΔ n \cdot d ) / λ \qquad ( 7 )$$

wherein (Δn) is the birefringence of the film, (d) is the thickness of the film and A is the wavelength of the incident beam.

The birefringence and accordingly optical retardation depends on the thickness of a film and the tilt angle of optical axis in the film (cf. Berek's compensator). Therefore, the skilled expert is aware that different optical retardations or different birefringence can be induced by adjusting the orientation of the liquid-crystalline molecules in the polymer film.

The birefringence (Δn) of the polymer film according to the present invention is preferably in the range from 0.18 to 0.80, more preferably from 0.20 to 0.70, very preferably from 0.25 to 0.55.

After photopolymerisation, the resulting polymer film can be removed from the substrate and combined with other substrates or optical films by a laminating process known by the skilled person. Suitable substrates and optical films are given above and include especially polarisers, in particular linear polarisers, photoalignment layers, or diffraction gratings, for example PB gratings.

The polymer film according to the present invention has good adhesion to plastic substrates, in particular to TAC, COP, and colour filters. Accordingly, it can be used as adhesive or base coating for subsequent polymerised RM layers or LC layers which otherwise would not well adhere to the substrates.

The polymer film of the present invention can also be used as alignment film or substrate for other liquid-crystalline or RM materials. The inventors have found that the polymer film obtainable from a RM formulation as described above and below, is in particular useful for multilayer applications due to its improved dewetting characteristics. In this way, stacks of optical films or preferably polymerised LC films can be prepared.

A preferred embodiment of the present invention relates to a process of preparing an optical element, preferably a diffraction grating, very preferably a PVH, PBG or Bragg PG, and to an optical element, preferably a diffraction grating, very preferably a PVH, PBG or Bragg PG, obtained by said process, wherein said process comprises the steps of:
A1) providing a first layer of an RM mixture or RM formulation according to the invention onto a substrate, preferably a substrate which has a surface grating or pattern, preferably by coating or printing,
A2) removing any solvents present,
A3) optionally annealing the first layer of the RM mixture or RM formulation, preferably at a temperature where it is in the nematic or chiral nematic phase,
A4) polymerising the RM mixture or RM formulation, preferably by exposure to UV light, under an inert atmosphere,
B1) providing a second layer of an RM mixture or RM formulation according to the invention on to the first layer, preferably by coating or printing,
B2) removing any solvents present,
B3) optionally annealing the second layer of the RM mixture or RM formulation, preferably at a temperature where it is in the nematic or chiral nematic phase,
B4) polymerising the RM mixture or RM formulation, preferably by exposure to UV light, under an inert atmosphere.

A third, fourth or further layers can be prepared by repeating process steps B1) to B4) using a different RM mixture or formulation.

The RM mixture or formulation of the first layer and the RM mixture or formulation of the second layer are preferably different from each other. In a preferred embodiment the RM mixtures used for preparation of the first and second layer, respectively, contain different amounts of a chiral compound(s) and/or contain chiral compounds with different HTP. As a consequence the helical pitch of the first and second layer will be different from each other. Alternatively, an RM mixture or RM formulation containing a chiral dopant (chiral mixture) is blended with an RM mixture or RM formulation that does not contain a chiral compound (achiral mixture), which allows to easily vary the amount of the chiral dopant in the blend of the chiral and achiral mixture, and thereby to easily adjust the helical pitch of the final layer and polymer film. First and second layers can then be prepared from such RM mixtures or blended RM mixtures. Very preferably the RM mixture or formulation of the second layer contains a higher amount of the same chiral compound than the RM mixture or formulation of the first layer, and/or the RM mixture or formulation of the second layer contains a chiral compound with a higher HTP than the RM mixture or formulation of the first layer. Preferably the helical pitch in the first layer is longer than the helical pitch in the second layer.

The RM mixtures and methods of the present invention do thus allow a simple way of preparing a multilayer of two or more chiral LC polymer films, by using one achiral RM host mixture comprising, or consisting of, one or more compounds of formula I, preferably one or more compounds of formula I, and optionally one or more compounds of formula DRM and/or MRM. This achiral RM host mixture can be used for the preparation of each individual layer. Chiral RM mixtures for use in the first, second or further layers, respectively, are prepared by adding different amounts of the same chiral compound to the RM host mixture, or by adding chiral compounds with differing HTP to the RM host mixture.

Preferably the component is a diffraction grating, very preferably a PBG or Bragg PG, comprising a polymer film obtained from an RM mixture or RM formulation according to the present invention as described above and below.

In summary, the polymer film and RM mixture according to the present invention are useful in optical elements like polarisers, compensators, alignment layer, circular polarisers or colour filters in liquid crystal displays or projection systems, decorative images, for the preparation of liquid crystal or effect pigments, and especially in reflective films with spatially varying reflection colours, e.g. as multicolour image for decorative, information storage or security uses, such as non-forgeable documents like identity or credit cards, banknotes etc.

The polymer film according to the present invention can be used in displays of the transmissive or reflective type. It can be used in conventional OLED displays or LCDs, in particular LCDs.

The present invention is described above and below with particular reference to the preferred embodiments. It should be understood that various changes and modifications might be made therein without departing from the spirit and scope of the invention.

Many of the compounds or mixtures thereof mentioned above and below are commercially available. All of these compounds are either known or can be prepared by methods which are known per se, as described in the literature (for example in the standard works such as Houben-Weyl, Methoden der Organischen Chemie [Methods of Organic Chemistry], Georg-Thieme-Verlag, Stuttgart), to be precise under reaction conditions which are known and suitable for said reactions. Use may also be made here of variants which are known per se, but are not mentioned here.

It will be appreciated that variations to the foregoing embodiments of the invention can be made while still falling within the scope of the invention. Alternative features serving the same, equivalent, or similar purpose may replace each feature disclosed in this specification, unless stated otherwise. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

Unless explicitly noted otherwise, all temperature values indicated in the present application, such as, for example, for the melting point T(K,N), the transition from the smectic (S) to the nematic (N) phase T(S,N) and the clearing point T(N,I), are quoted in degrees Celsius (° C.). Furthermore, K denotes the crystalline state, N denotes the nematic phase, and I denotes the isotropic phase. The data between these symbols represent the transition temperatures.

Unless explicitly noted otherwise, all physical properties have been and are determined according to “Merck Liquid Crystals, Physical Properties of Liquid Crystals”, Status November 1997, Merck KGaA, Germany and are given for a temperature of 20° C., unless explicitly stated otherwise.

Above and below, percentages are percent by weight unless stated otherwise. All temperatures are given in degrees Celsius.

Above and below, m.p. denotes the melting point, T_{NI} and cl.p. denote the nematic-isotropic phase transition temperature (or clearing point), T_{g} denotes glass transition temperature. Furthermore, C denotes the crystalline state, N denotes the nematic phase, S_{A}, S_{B} etc. denotes the smectic A phase, smectic B phase etc., S_{X} denotes an unidentified smectic phase, X denotes an unidentified mesophase and I denotes the isotropic phase. The values between these symbols represent the transition temperature in ° C. Unless stated otherwise, the onset temperature is given for T_{NI}. Δn denotes the optical anisotropy or birefringence (Δn=nₑ−nₒ, where nₒ denotes the refractive index perpendicular to the longitudinal molecular axes and nₑ denotes the refractive index parallel thereto) at 589 nm and room temperature. The optical and electro optical data are measured at 20° C., unless expressly stated otherwise.

“Clearing point” and “clearing temperature” mean the temperature of the transition from an LC phase into the isotropic phase. “MEK” means methyl ethyl ketone, “MIBK” means methyl isobutyl ketone, “Cyclopent” means cyclopentanone, “DCM” means dichloromethane and “IPA” means isopropyl alcohol.

Unless stated otherwise, the percentages of individual solid compounds in an RM mixture as described above and below refer to the total amount of solids in the mixture, i.e., without any solvents.

Unless stated otherwise, all optical, electro optical properties and physical parameters like birefringence, permittivity, electrical conductivity, electrical resistivity and sheet resistance, refer to a temperature of 20° C.

The invention will now be described in more detail by reference to the following working examples, which are illustrative only and do not limit the scope of the invention.

### Example 1

Compound I1 is synthesized as described below.

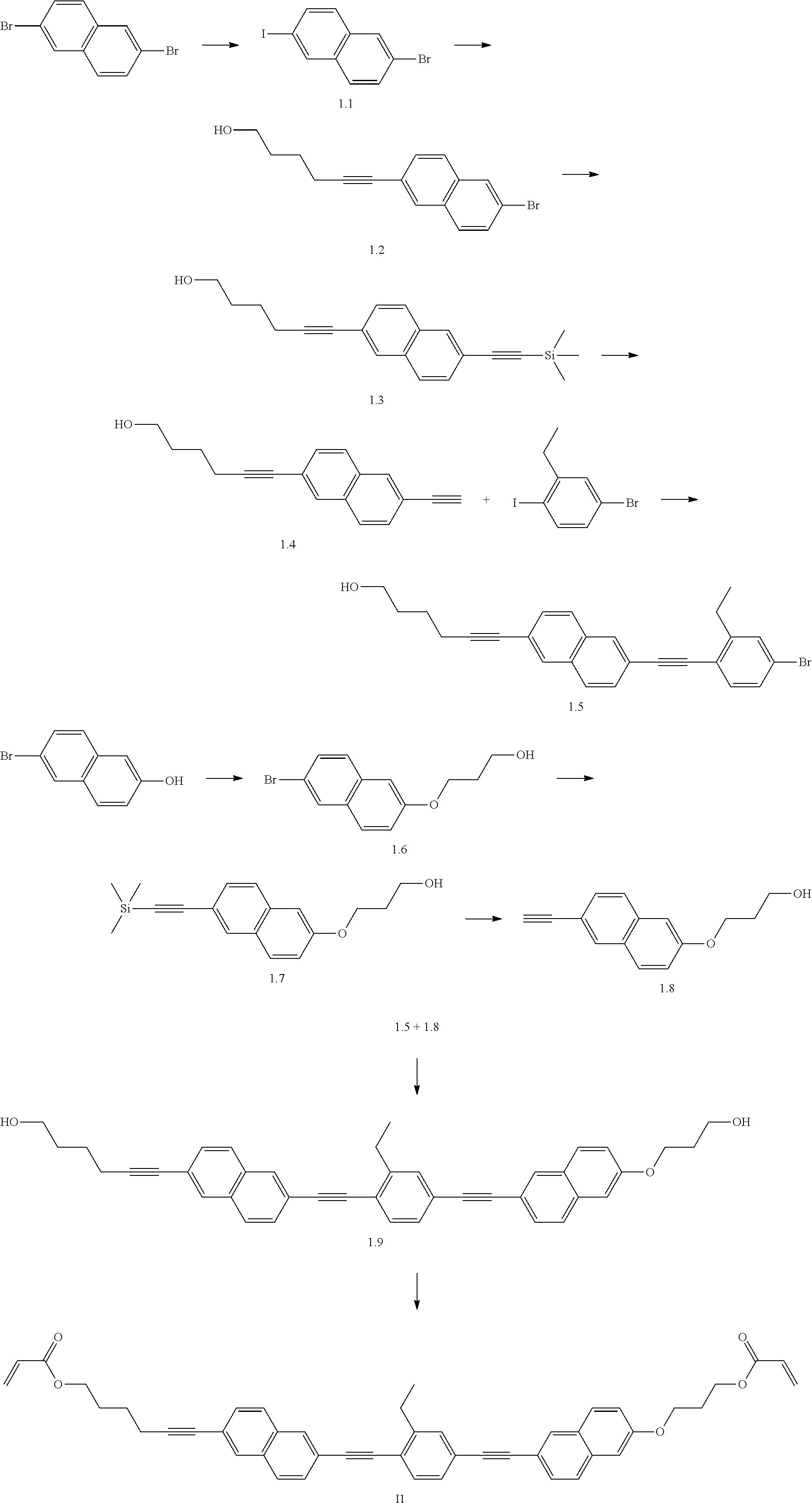

### 1.1 Step 1

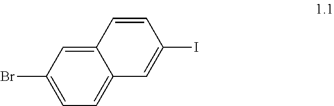 [SMILES: Brc1ccc2cc(I)ccc2c1]

To a solution of 2,6-dibromonaphthalene (23.5 g; 0.082 moles) in anhydrous THE (700 ml), cooled to <−65° C. (whereupon a precipitate is observed), is added dropwise n-butyllithium (2.5 M in hexanes; 40 ml; 0.1 moles) over 10 minutes keeping t<−65° C. A solution forms during addition and is then left for a further 1.5 hours at −70° C. Now a solution of iodine (24.6 g; 0.097 moles) in anhydrous THE (300 ml) is added dropwise over 30 minutes keeping the temperature at <−65° C. After a further hour at −70° C., the cooling bath is removed and the dark mixture allowed to warm up to 0° C. (circa 1 hour). The mixture is quenched over a couple of minutes with a saturated solution of sodium metabisulphite. The reaction temperature reaches 5° C. The layers are separated, the organic layer washed with sodium metabisulphite solution, twice with brine, dried over sodium sulphate and evaporated in vacuo to afford crude product as an orange-brown solid (28 g). This is recrystallized from 350 ml ethyl acetate, leaving in the freezer overnight. The mixture is filtered, washed twice with ex-freezer ethyl acetate and dried in vacuo at 40° C. to afford 1.1 as pale tan solid (15.55 g; 57%).

NMR analysis reveals the batch contains circa 5% by moles of the 2,6-dibromonaphthalene starting material. This is used in the next step without further purification.

### 1.2 Step 2

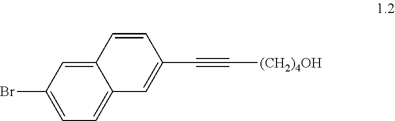

To a degassed solution of 1.1 (6.0 g; 0.018 moles) and 5-hexyn-1-ol (1.95 g; 0.02 moles) in diisopropylamine (30 ml) and THE (80 ml), over a cold water bath is added copper I iodide (80 mg) and bis-(triphenylphosphine)palladium II chloride (160 mg). The mixture is stirred at room temperature for 30 minutes then heated to 30° C. for 2 hours, cooled, evaporated in vacuo, slurried in ethyl acetate then filtered and the filtrate evaporated in vacuo then azeotroped with petroleum ether. The residue is chromatographed on silica (225 g), eluting with DCM followed by 15% ethyl acetate in DCM to afford 1.2 as a brown oil (4.45 g; 81%).

### 1.3 Step 3

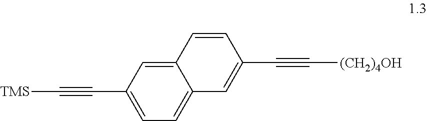

To a degassed solution of 1.2 (4.45 g; 0.015 moles) and TMS acetylene (2.13 g; 0.022 moles) in diisopropylamine (30 ml) and THE (20 ml) is added copper I iodide (55 mg), tri-(tert-butylphosphonium)tetrafluoroborate (140 mg) then palladium II acetate (110 mg). The mixture is heated to 45° C. for 1 hour, cooled, evaporated in vacuo, slurried in ethyl acetate then filtered and the filtrate evaporated in vacuo then azeotroped with DCM/petroleum ether. The residue is chromatographed on silica (150 g) eluting with DCM followed by 15% ethyl acetate in DCM to afford 1.3 as a brown oil (circa 6 g; solvent not completely removed).

### 1.4 Step 4

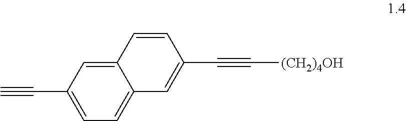

To a degassed solution of 1.4 (circa 6 g wet) in methanol (130 ml) is added potassium carbonate (0.15 g; 0.0011 moles). The mixture is stirred at 17° C. overnight, evaporated in vacuo, azeotroped DCM/petroleum ether then layered in DCM (some insoluble present) onto silica (100 ml), eluting with 10% ethyl acetate in DCM to afford crude product. This is dissolved in DCM (10 ml) to which is added petroleum ether (100 ml) and the resultant mixture cooled to −20° C. then filtered, washed petroleum ether and dried in vacuo at 30° C. to afford 1.4 as a pale yellow solid (2.82 g, 77% from bromide 1.2).

### 1.5 Step 5

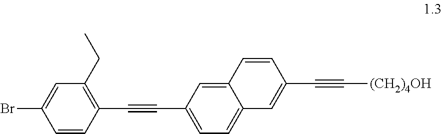

To a degassed solution of 1.4 (2.82 g; 0.011 moles) and 4-bromo-2-ethyliodobenzene (4.35 g; 0.014 moles) in diisopropylamine (30 ml)), over a cold water bath is added copper I iodide (80 mg) and bis(triphenylphosphine)palladium II chloride (160 mg). The mixture is stirred at room temperature for 10 minutes then heated to 30° C. for 10 minutes then 35° C. for 40 minutes. After cooling, the mixture is evaporated in vacuo, slurried in ethyl acetate then filtered and the filtrate evaporated in vacuo and azeotroped with petroleum ether. The orange solid residue is chromatographed on silica (125 g), eluting with DCM followed by 12% ethyl acetate in DCM to afford crude product. 1.5 is afforded as a cream/yellow solid following recrystallization of this from 20 ml DCM/150 ml petroleum ether at −20° C. (4.17 g; 85%).

### 1.6 Step 6

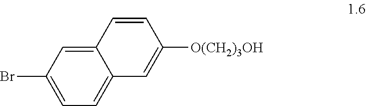

2-Bromo-6-hydroxynaphthalene (22.3 g, 100 mmol), 3-bromopropanol (15.3 g, 110 mmol), potassium carbonate (16.6 g, 120 mmol), butanone (60 ml) and sodium iodide (3 g, 20 mmol) are heated at 80° C. overnight. The mixture is cooled and diluted with acetone. The solid is filtered off and washed with acetone. The solvent from the filtrate is removed in vacuo. The solid is recrystallized from IPA (90 ml) and water (90 ml). The mixture is left in the fridge for 1 hour. The solid is filtered off, washed with IPA:water 1:1 to give the desired product 2-bromo-6-(3-hydroxypropoxy)naphthalene 1.6 as a white solid (22.2 g, 79% yield).

### 1.7 Step 7

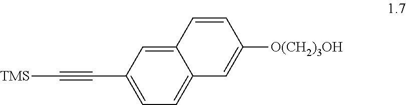

To a degassed solution of 2-bromo-6-(3-hydroxypropoxy)naphthalene (1.6; 24.3 g; 0.086 moles) and TMS acetylene (13.1 g; 0.134 moles) in diisopropylamine (100 ml) and THE (70 ml) is added copper I iodide (220 mg), tri-(tert-butylphosphonium)tetrafluoroborate (550 mg) then palladium II acetate (440 mg). The mixture is heated to 45° C. for 1 hour, cooled, evaporated in vacuo, then stirred in ethyl acetate and filtered. After evaporation of the filtrate in vacuo, the residue is dissolved in DCM the chromatographed on silica (450 ml) eluting with DCM the 20% ethyl acetate in DCM to afford 1.7 as a tan solid (24.6 g; 95%).

### 1.8 Step 8

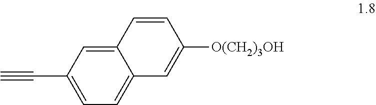

To a degassed solution of 1.7 (24.6 g; 0.083 moles) in methanol (680 ml) is added potassium carbonate (1.06 g; 0.0077 moles). The mixture is stirred at 17° C. overnight, evaporated in vacuo, azeotroped DCM/petroleum ether then layered in DCM (some insoluble present) onto silica (200 ml), then eluted with 20% ethyl acetate in DCM to afford crude product. This is dissolved in DCM (50 ml) to which is added petroleum ether (300 ml) then filtered, washed 6:1petroleum ether:DCM then petroleum ether and dried in vacuo at 35° C. to afford 1.8 as a tan solid (14.55 g, 78%).

### Step 9

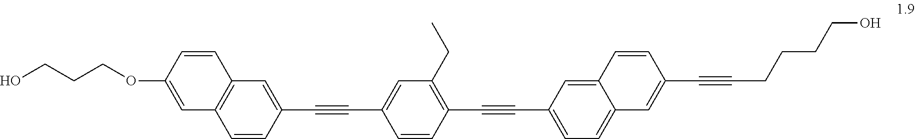 [SMILES: CCc1cc(C#Cc2ccc3cc(OCCCO)ccc3c2)ccc1C#Cc1ccc2cc(C#CCCCCO)ccc2c1]

To a degassed solution of 1.5 (5.87 g; 0.014 moles) and 1.8 (3.08 g; 0.014 moles) in diisopropylamine (45 ml) and THE (20 ml) is added copper I iodide (220 mg), tri-(tert-butylphosphonium)tetrafluoroborate (340 mg) then palladium II acetate (280 mg). The mixture is heated to 45° C. for 1.25 hours, cooled, evaporated in vacuo, then stirred vigorously in ethyl acetate (100 ml) and 2M hydrochloric acid (250 ml). Petroleum ether (100 ml) is added then the mixture is filtered and washed several times with water and 1:1 ethyl acetate:petroleum ether. The wet solid is washed twice with IMS, once with ether, once with 1:1 ether:petroleum ether and finally with petroleum ether, before drying in vacuo at 50° C. to afford 1.9 as a tan solid (7.10 g; 91%).

### Step 10

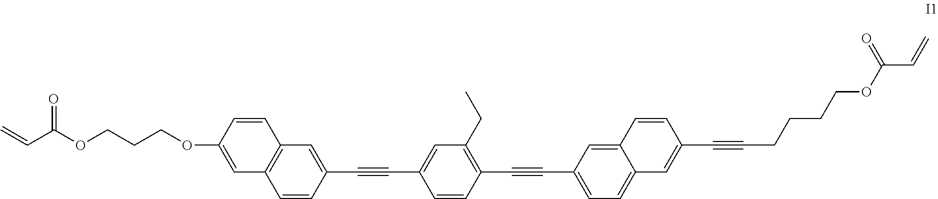 [SMILES: C=CC(=O)OCCCCC#Cc1ccc2cc(C#Cc3ccc(C#Cc4ccc5cc(OCCCOC(=O)C=C)ccc5c4)cc3CC)ccc2c1]

To an ice/water bath-cooled slurry of 1.9 (7.10 g; 0.0123 moles) in anhydrous DCM (225 ml) and triethylamine (10.3 ml; 0.074 moles) is added dropwise over 15 minutes 3-chloropropionyl chloride (2.94 g; 0.031 moles). After 30 minutes, tlc indicated complete reaction, so trimethylamine (20 ml) and some Irganox are added and the solution heated to 38° C. overnight, cooled and evaporated in vacuo. The residue is dissolved in DCM (100 ml), petroleum ether (100 ml) is added and the mixture filtered and washed through with 1:1 DCM:petroleum ether. The filtrate is evaporated in vacuo then re-dissolved in DCM (70 ml). Petroleum ether (70 ml) is added and the slightly turbid solution is chromatographed on silica (175 g) eluting with 1:1 DCM:petroleum ether then DCM to yield crude product as a cream solid. This is dissolved in DCM (45 ml) to which petroleum ether (100 ml) is added. Rapid crystallisation ensued then a further 100 ml of petroleum ether is added. After a few minutes the slurry is filtered, washed with 4:1 petroleum ether:DCM then petroleum ether to afford I1 as a lemon-yellow solid after drying in vacuo at 35° C. (5.96 g; 71%).

The compound I1 has a refractive index nₑ of 2.349, measured in the LC host formulation H1 at 445 nm and room temperature.

The host formulation H1 has the following composition:

| N1 | % Composition |
|---|---|
| Irganox ®1076 | 0.17% |
| TR-PBG-345 | 3.16% |
| Polyfox ™656 | 0.42% |
| MRM1a | 5.16% |
| DRMa1b | 8.32% |
| MRM1b | 5.16% |
| DRMa1a | 32.04% |
| MRM8a | 45.58% |
| Total | 100% |
| Solvent | 80% |
| MEK:Cyclopentanone:MIBK:2- | |
| Undecanone | |
| 1:2:1:2 | |
| Solids % | 20% |
| Total | 100% |

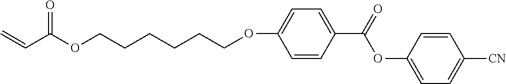 [SMILES: C=CC(=O)OCCCCCCOc1ccc(C(=O)Oc2ccc(C#N)cc2)cc1]

MRM1a 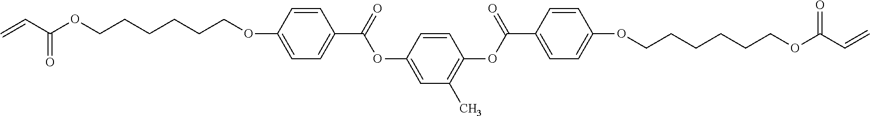 [SMILES: C=CC(=O)OCCCCCCOc1ccc(C(=O)Oc2ccc(OC(=O)c3ccc(OCCCCCCOC(=O)C=C)cc3)c(C)c2)cc1]
DRMa1b 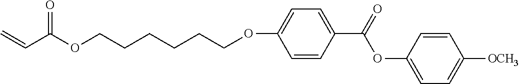 [SMILES: C=CC(=O)OCCCCCCOc1ccc(C(=O)Oc2ccc(OC)cc2)cc1]
MRM1b 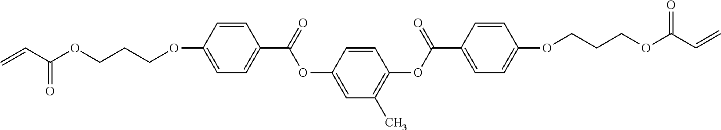 [SMILES: C=CC(=O)OCCCOc1ccc(C(=O)Oc2ccc(OC(=O)c3ccc(OCCCOC(=O)C=C)cc3)c(C)c2)cc1]
DRMa1a 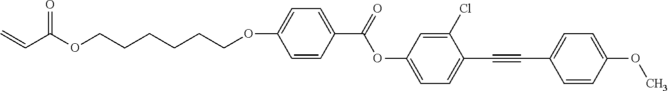 [SMILES: C=CC(=O)OCCCCCCOc1ccc(C(=O)Oc2ccc(C#Cc3ccc(OC)cc3)c(Cl)c2)cc1]
MRM8a

Irganox®1076 is a stabilizer being commercially available (Ciba). TR-PBG 345 is a photoinitiator being commercially available (Tronly). Polyfox™PF-656 is a surfactant being commercially available (Synthomer).

### Examples 2-25

The following compounds are prepared in analogy to the method described in Example 1. The nₑ values are measured in LC host formulation H1 at 445 nm and room temperature.

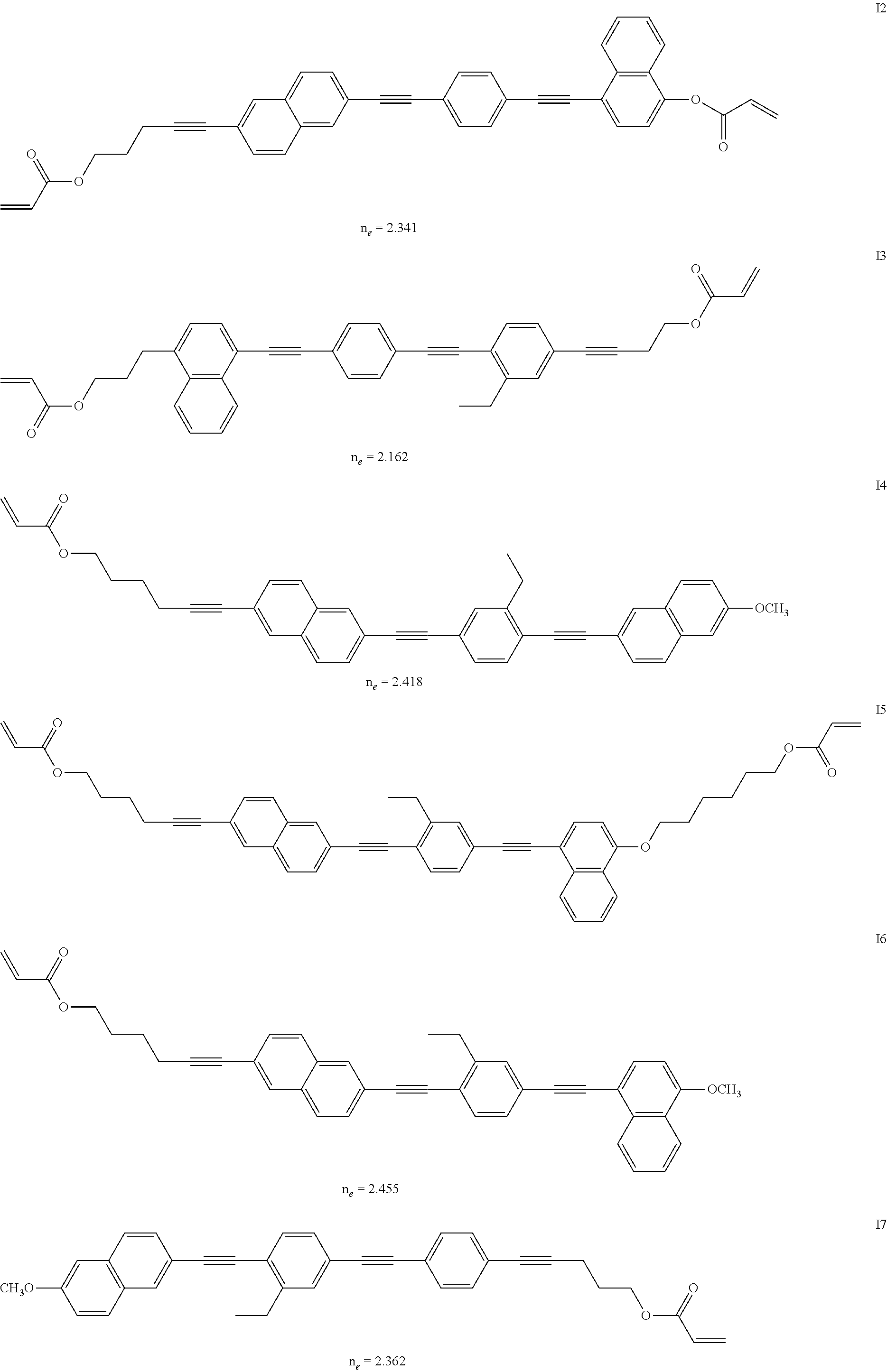

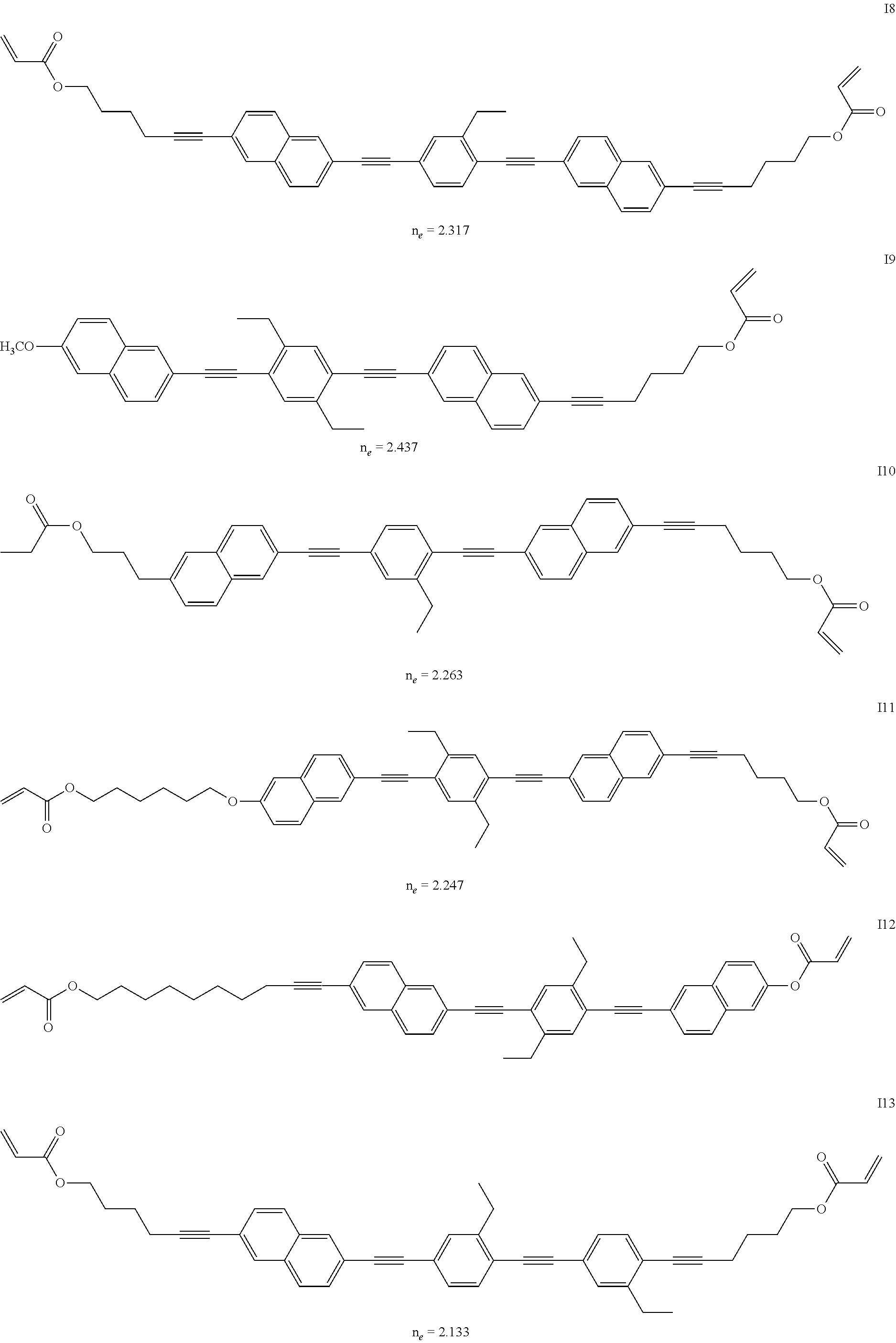

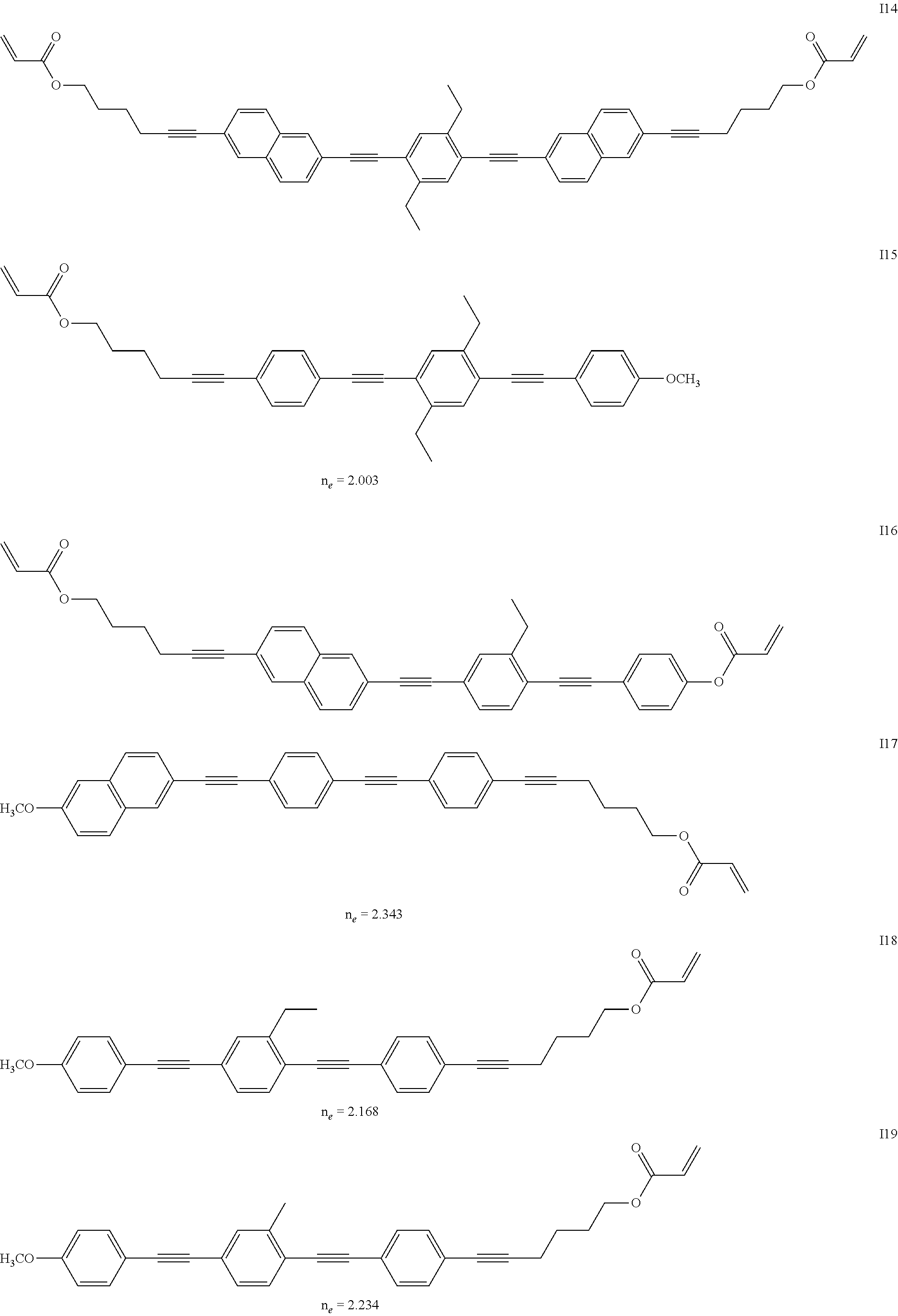

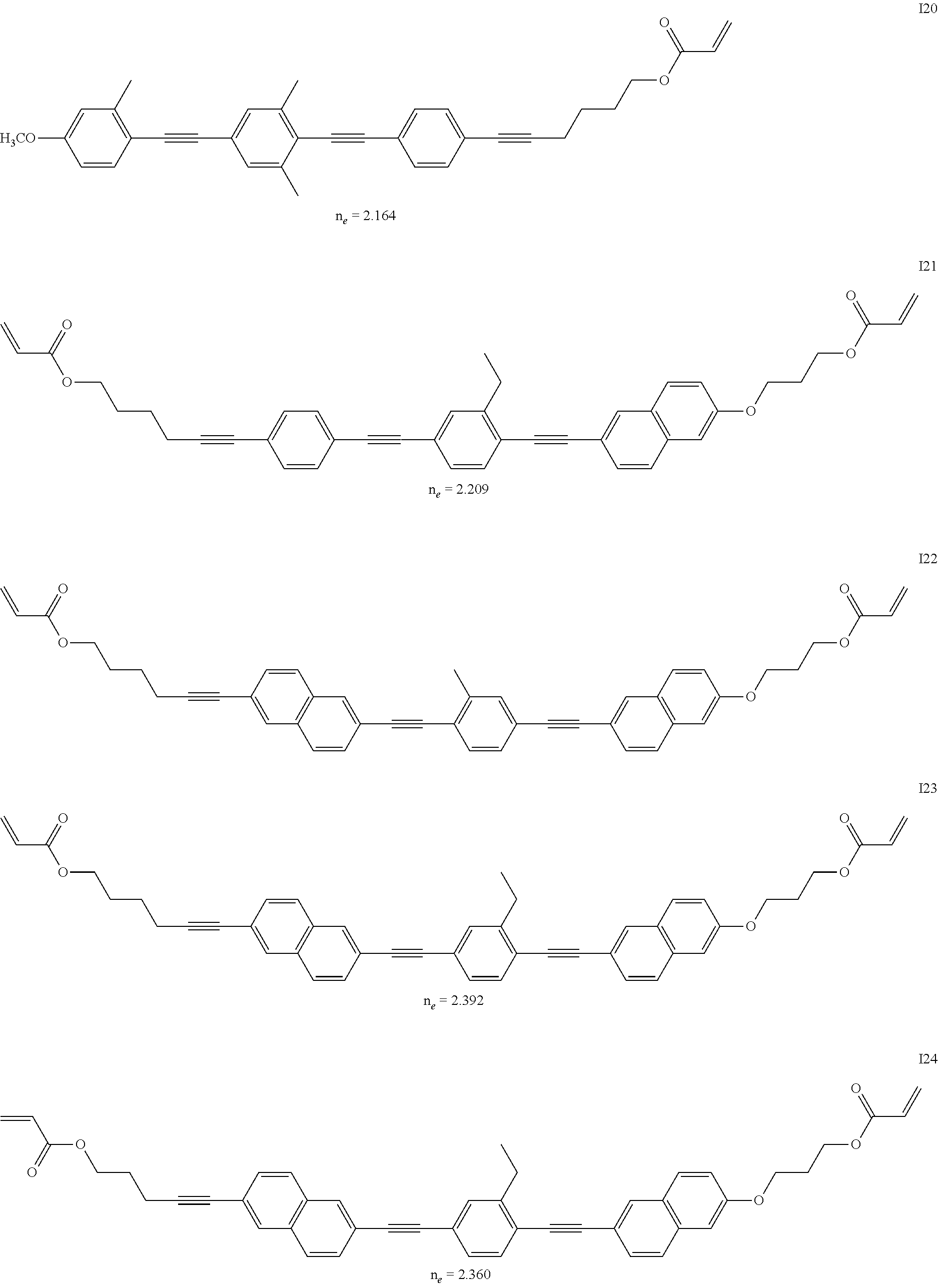

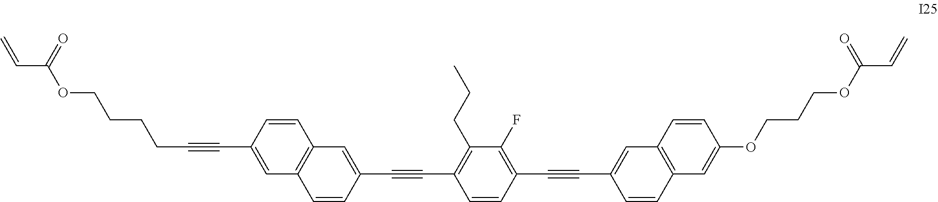 [SMILES: C=CC(=O)OCCCCC#Cc1ccc2cc(C#Cc3ccc(C#Cc4ccc5cc(OCCCOC(=O)C=C)ccc5c4)c(F)c3CCC)ccc2c1]

### RM Mixture, RM Formulation and Polymer Film Examples

In the following examples, unless stated otherwise, measurements are carried out as follows:

### Refractive Indices

Refractive Indices are measured with the Metricon equipment.

Polymer films are prepared by spin coating an RM formulation onto rubbed polyimide glass with a thickness which can range from 600 nm to 1 um. Refractive indices are measured using the Metricon prism coupler method. Both extraordinary (ne) and ordinary refractive indices (no) are measured at three wavelengths of 445 nm, 520 nm and 638 nm. Data is extrapolated for nₑ and nₒ at 620 nm for all measured films using Cauchy's fit.

### Colouring, Absorption

Colouring is measured with a Spectrophotometer i5 (x-rite). This uses the CIE, or Commission Internationale de l'clairage (translated as the International Commission on Illumination) standardized color order systems for describing color. The CIE Color Systems utilize three coordinates to locate a color in a color space. These color spaces include CIE L*a*b. To obtain these values, the instrument perceives the reflected light wavelengths as numeric values. When a color is expressed in CIELAB, L* defines lightness, a* denotes the red/green value and b* the yellow/blue value. An increased b* colour represents a shift toward yellow.

In the following examples the b* value is measured to evaluate the colouring of the RM mixture or polymer film.

The b* value can be also used as an indicator for the absorption of the material, e.g. the RM mixture or the polymer film. A lower b* value indicates a lower absorbing material.

Polymer films with a thickness of 1 um are prepared by spin coating an RM formulation onto rubbed polyimide glass. Once spin coated, the wet layer is heated on a hot stage at 80° C. for 60 s to evaporate the solvent and anneal the RM layer.

Before the irradiation step (polymerisation step) the RM layer is purged in a nitrogen environment for 1 minute. Following that, the RM layer is exposed to unpolarised UV light of 250-400 nm from a UV Hg lamp in an nitrogen environment at 40° C. for 60 seconds to fully polymerise the RM layer and form a polymer film.

The b* value of the polymer film is then measured using a Benchtop Spectrophotometer i5.

Absorption of the polymer film can be measured with the Cary 7000 UMS equipment.

### Comparative Example 1

Reference RM mixture R1 is formulated as follows:

| C1 | % Composition |
|---|---|
| Irganox ®1076 | 0.08% |
| Fluor N 562 | 0.60% |
| Irgacure ®651 | 1.00% |
| DRMa1a | 39.40% |
| MRM8a | 49.20% |
| DRMa1b | 9.72% |
| Total | 100% |

Irgacure®651 is a photoinitiator being commercially available (Ciba). Fluor N 562 is a surfactant being commercially available (Cytonix).

Reference mixture R1 does not contain a compound of formula I according to the present invention. Reference mixture R1 exhibits a T_{NI} of 130° C.

Reference RM formulation RF1 is formulated by dissolving reference mixture R1 in the solvent blend MEK:Cyclopentanone:MIBK:2-Undecanone=1:2:1:2 at a concentration of 20% solids.

A polymer film is prepared from reference formulation RF1 and its refractive indices and b* value are measured as described above.

The refractive indices are as follows:

| Wavelength/nm | nₑ | nₒ |
|---|---|---|
| 445 | 1.8073 | 1.57444 |
| 520 | 1.7663 | 1.56179 |
| 638 | 1.7365 | 1.54506 |
| 620 (extrapolated) | 1.74 | 1.574 |

The polymer film of reference mixture R1 does only show moderate nₑ values.

The b* value of the polymer film is 1.43, meaning that the polymer film shows weak colouring which indicates low absorption.

### Comparative Example 2

Reference RM mixture R2 is formulated as follows:

| R2 | % Composition |
|---|---|
| Irganox ®1076 | 0.08% |
| TR-PBG 345 | 1.5% |
| Polyfox PF656 | 0.36% |
| DRMf1 | 25.50% |
| MRM10a | 50.99% |
| DRMk1 | 21.57% |
| Total | 100% |

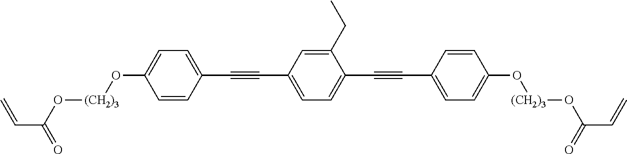

DRMf1 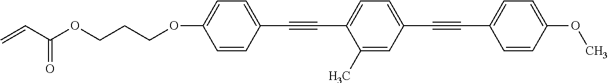 [SMILES: C=CC(=O)OCCCOc1ccc(C#Cc2ccc(C#Cc3ccc(OC)cc3)cc2C)cc1]
MRM10a 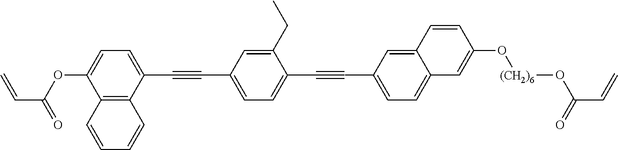
DRMm1

Reference mixture R2 does not contain a compound of formula I according to the present invention. Reference mixture R2 exhibits a T_{NI} of 175° C.

Reference RM formulation RF2 is formulated by dissolving reference mixture R2 in the solvent blend MEK:Cyclopentanone:MIBK:2-Undecanone=1:2:1:2 at a concentration of 15% solids.

A polymer film is prepared from reference formulation RF2 and its refractive indices and b* value are measured as described above.

The refractive indices are as follows:

| Wavelength/nm | nₑ | nₒ |
|---|---|---|
| 445 | 2.106 | 1.611 |
| 520 | 1.983 | 1.588 |
| 638 | 1.91 | 1.568 |
| 620 (extrapolated) | 1.917 | 1.571 |

The polymer film of reference mixture R2 shows higher nₑ values than the polymer film of reference mixture R1.

However, compared to the polymer film of rmixture R1 the b* value of the polymer film of mixture R2 is 3.89 and thus much higher, meaning that the polymer film shows undesired strong colouring which indicates undesired high absorption.

### Example M1

Achiral RM mixture M1 is formulated as follows:

| M1 | % Composition |
|---|---|
| Irganox ® 1076 | 0.25% |
| TR-PBG-345 | 1.50% |
| MRM10a | 22.60% |
| I1 | 51.58% |
| I7 | 24.07% |
| Total | 100.00% |

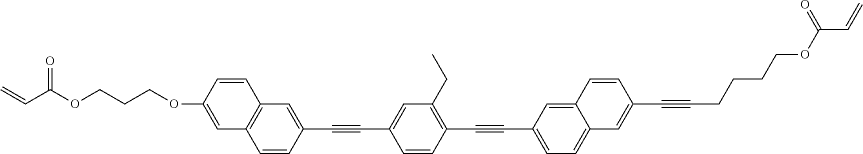 [SMILES: C=CC(=O)OCCCCC#Cc1ccc2cc(C#Cc3ccc(C#Cc4ccc5cc(OCCCOC(=O)C=C)ccc5c4)cc3CC)ccc2c1]

I1 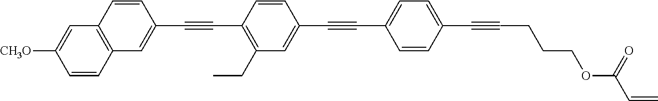
I7

Mixture M1 contains the compounds I1 and I7 of formula I according to the present invention. Mixture M1 exhibits a T_{NI} of 204° C.

RM formulation F1 is formulated by dissolving mixture M1 in the solvent blend MEK:Cyclopentanone:MIBK:2-Undecanone=1:2:1:2 at a concentration of 20% solids.

A polymer film is prepared from formulation F1 and its refractive indices and b* value are measured as described above.

The refractive indices are as follows:

| Wavelength/nm | nₑ | nₒ |
|---|---|---|
| 445 | 2.272 | 1.613 |
| 520 | 2.097 | 1.585 |
| 638 | 2.001 | 1.565 |
| 620 (extrapolated) | 2.009 | 1.567 |

The polymer film of mixture M1 shows higher nₑ values than the polymer films of reference mixture R1 and R2.

The b* value of the polymer film is 2.29, meaning that the polymer film shows weak colouring which indicates low absorption.

The polymer film of mixture M1 shows both high nₑ values and weak colouring indicating low absorption, as compared to the polymer films of reference mixtures R1 and R2 which show either undesired low nₑ values or undesired strong colouring. It is therefore very suitable for AR/VR applications where low absorption and high birefringence are required, especially AR applications such as those using PVH technology.

### Example M2

Achiral RM mixture M2 is formulated as follows:

| M2 | % Composition |
|---|---|
| Irganox ® 1076 | 0.25% |
| TR-PBG-345 | 1.50% |
| Polyfox ™PF-656 | 0.38% |
| MRM10a | 16.83% |
| I1 | 51.68% |
| I17 | 29.36% |
| Total | 100.000% |

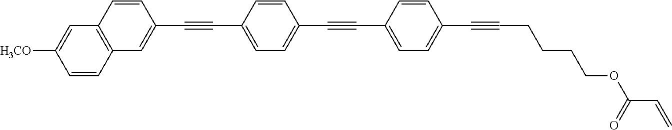 [SMILES: C=CC(=O)OCCCCC#Cc1ccc(C#Cc2ccc(C#Cc3ccc4cc(OC)ccc4c3)cc2)cc1]

I17

Mixture M2 contains the compounds I1 and I17 of formula I according to the present invention. Mixture M2 exhibits a T_{NI} of 200° C.

RM formulation F2 is formulated by dissolving mixture M2 in the solvent blend MEK:Cyclopentanone:MIBK:2-Undecanone=1:2:1:2 at a concentration of 20% solids.

A polymer film is prepared from formulation F2 and its refractive indices and b* value are measured as described above.

The refractive indices are as follows:

| Wavelength/nm | nₑ | nₒ |
|---|---|---|
| 445 | 2.269 | 1.612 |
| 520 | 2.095 | 1.584 |
| 638 | 1.999 | 1.564 |
| 620 (extrapolated) | 2.007 | 1.566 |

The polymer film of mixture M2 shows higher nₑ values than the polymer films of reference mixture R1 and R2.

The b* value of the polymer film is 2.65, meaning that the polymer film shows weak colouring which indicates low absorption.

The polymer film of mixture M2 shows both high nₑ values and weak colouring indicating low absorption, as compared to the polymer films of reference mixtures R1 and R2 which show either undesired low nₑ values or undesired strong colouring. It is therefore very suitable for AR/VR applications where low absorption and high birefringence are required, especially AR applications such as those using PVH technology.

### Example M3

Chiral RM mixture M3 is formulated as follows:

| M3 | % Composition |
|---|---|
| Irganox ® 1076 | 0.25% |
| TR-PBG-345 | 1.50% |
| Polyfox ™PF-656 | 0.38% |
| CRM-1-1-6a | 10.000% |
| MRM10a | 15.11% |
| I1 | 46.40% |
| I17 | 26.36% |
| Total | 100.000% |

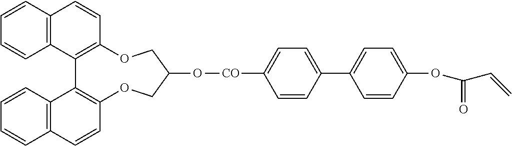 [SMILES: C=CC(=O)Oc1ccc(-c2ccc(C(=O)OC3COc4ccc5ccccc5c4-c4c(ccc5ccccc45)OC3)cc2)cc1]

CRM1-1-6a

### Example M4

Achiral RM mixture M4 is formulated as follows:

| M4 | % Composition |
|---|---|
| Irganox ® 1076 | 0.25% |
| TR-PBG-345 | 1.50% |
| MRM10a | 16.90% |
| I1 | 51.87% |
| I17 | 29.48% |
| Total | 100.000% |

### Example M5

Chiral RM mixture M5 is formulated as follows:

| M5 | % Composition |
|---|---|
| Irganox ® 1076 | 0.25% |
| TR-PBG-345 | 1.50% |
| CRM1-1-6a | 10.000% |
| MRM10a | 15.18% |
| I1 | 46.60% |
| I17 | 26.47% |
| Total | 100.000% |

### Example M6

Two formulation blends B1 and B2 of the above mixtures M2 to M5 are prepared as follows:

| B1 | Composition |
|---|---|
| M5 | 0.40 |
| M4 | 0.61 |
| Solvent | 1.01 |
| MEK:Cyclopentanone:MIBK:2- | |
| Undecanone = 1:2:1:2 | |

| B2 | Composition |
|---|---|
| M3 | 0.72 |
| M2 | 0.09 |
| Solvent | 0.81 |
| MEK:Cyclopentanone:MIBK:2- | |
| Undecanone = 1:2:1:2 | |

From the above 2 formulations B1 and B2 a first layer L1 and second layer L2, respectively, are provided on a PVH grating with a PB pitch of 420 nm as follows:

For the first layer L1, the blended formulation B1 is spin-coated at 2500 rpm on a PVH substrate with a wet layer thickness of ~1 mm. The wet layer is heated on a hot stage at 80° C. for 60 s to evaporate the solvent and anneal the RM layer.

Before the irradiation step (polymerisation step) the RM layer is purged in a nitrogen environment for 1 minute. Following that, the RM layer is exposed to unpolarised UV light of 250-400 nm from a UV Hg lamp in a nitrogen environment at 40° C. for 60 seconds to fully polymerise the RM layer and form a polymer film.

FIG. 1a (×20 magnification) and FIG. 1b (×100 magnification) show microscopic images of the PVH grating coated with layer L1. It can be seen that the coated and polymerised RM layer shows uniform alignment with no dewetting or defects in the PVH, and in FIG. 1b the stripes of the PVH grating are clearly visible through the RM layer.

After coating the first layer L1, the second RM layer L2 is spin-coated onto the first layer L1 using mixure B2 at 900 rpm. The wet layer is heated on a hot stage at 80° C. for 60 s to evaporate the solvent and anneal the RM layer.

Before the irradiation step (polymerisation step) the RM layer is purged in a nitrogen environment for 1 minute. Following that, the RM layer is exposed to unpolarised UV light of 250-400 nm from a UV Hg lamp in a nitrogen environment at 40° C. for 60 seconds to fully polymerise the RM layer and form a polymer film.

FIG. 2a (×20 magnification) and FIG. 2b (×100 magnification) show microscopic images of the PVH grating coated with layer L1 and layer L2. It can be seen that the coated and polymerised RM layer shows uniform alignment with no dewetting or defects in the PVH when overcoated, and in FIG. 2b the stripes of the PVH grating are clearly visible through the RM layers.

This is an important achievement with an RM mixture which exhibits a high refractive index and a low absorption.

1. A compound of formula I 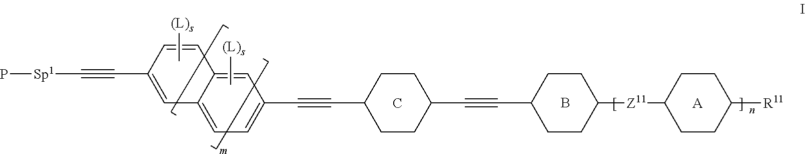
wherein the individual radicals, independently of each other and on each occurrence identically or differently, have the following meanings
P a polymerisable group,
Sp a spacer group or a single bond,
Sp¹ a spacer group or a single bond,
R¹¹ H, F, Cl, CN, CH═CH₂, alkyl, alkoxy, thioalkyl, alkylcarbonyl, alkoxycarbonyl, alkylcarbonyloxy or alkoxycarbonyloxy with 1 to 6 C atoms which is optionally fluorinated or chlorinated, or P-Sp,
A, B phenylene-1,4-diyl, naphthalene-1,4-diyl, naphthalene-2,6-diyl, fluorene-2,7-diyl, dibenzothiophene-2,7-diyl, dibenzofuran-2,7-diyl, benzo[1,2-b:4,5-b′]dithiophene-2,5-diyl, indole-4,7-diyl, benzothiophene-4,7-diyl, 9,10-dihydro-phenanthrene-2,7-diyl or 1,2,3,4-tetrahydronaphthalene-5,8-diyl where, in addition, one or more CH groups in these groups may be replaced by N, all of which are optionally substituted by one or more groups L or P-Sp-,
C phenylene-1,4-diyl or naphthalene-2,6-diyl, preferably phenylene-1,4-diyl or naphthalene-2,6-diyl, in which, in addition, one or more CH groups in these groups may be replaced by N, and which are optionally substituted by one or more groups L or P-Sp-,
provided that not more than one of A, B and C may denote naphthalene-1,4-diyl,
L F, Cl, —CN, —SCN, P-Sp-, or straight chain alkyl having 1 to 25 C atoms or branched or cyclic alkyl having 3 to 25 atoms, wherein one or more non-adjacent CH₂-groups are optionally replaced by —O—, —S—, —CO—, —CO—O—, —O—CO—, —O—CO—O—, CR⁰═CR⁰⁰—, —C≡C—, 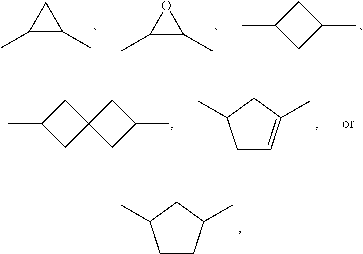
in such a manner that O- and/or S-atoms are not directly connected with each other, and wherein one or more H atoms are each optionally replaced by P-Sp-, F or Cl, or two substituents L that are connected to directly adjacent C atoms may also form a cycloalkyl or cycloalkenyl group with 5, 6, 7 or 8 C atoms,
Z¹¹ —O—, —S—, —CO—, —COO—, —OCO—, —S—CO—, —CO—S—, —O—COO—, —CO—NR⁰—, —NR⁰—CO—, —NR⁰—CO—NR⁰⁰, —NR⁰—CO—O—, —O—CO—NR⁰—, —OCH₂—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CR⁰—, —CY¹═CY²—, —C≡C—, —CH═CH—COO—, —OCO—CH═CH— or a single bond, preferably —COO—, —OCO—, —C≡C—, or a single bond,
n1 1, 2, 3 or 4,
s 0, 1, 2 or 3,
R⁰, R⁰⁰ H or alkyl having 1 to 12 C atoms,
Y¹, Y² H, F, Cl, NCS, or CN,
m 0 or 1,
n 0, 1, 2, 3 or 4.

2. The compound according to claim 1, wherein rings A and B are selected from the group consisting of phenylene-1,4-diyl, naphthalene-1,4-diyl and naphthalene 2,6-diyl, all of which are optionally substituted by one or more groups L and/or P-Sp.

3. The compound according to claim 1, wherein P is acrylate or methacrylate, and Sp¹ is —(CH₂)ₛ₁—, in which s1 is an integer from 1 to 12.

4. The compound according to claim 1, wherein m=0 and n=0 and the rings B and C together with the 1,4-phenylene group form a group selected from the following formulae or their mirror images, wherein * denotes the linkage to the acetylene group in formula I: 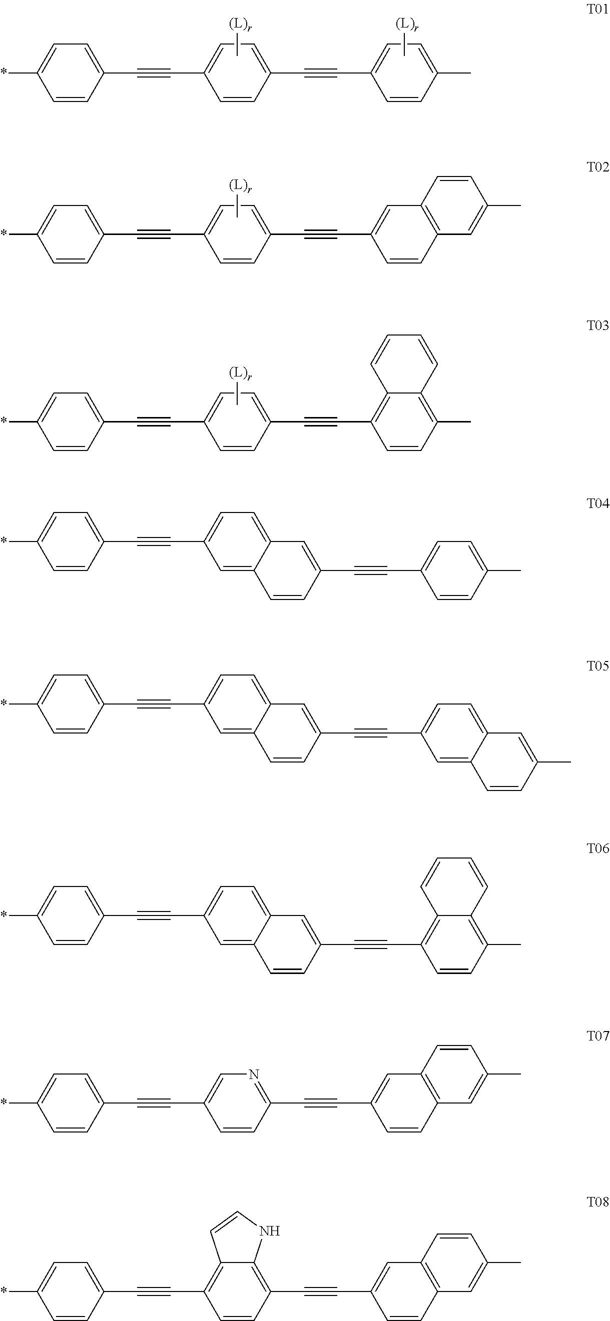
wherein the naphthalene groups are optionally substituted with one or two groups L, r is 0, 1, 2, 3 or 4, and L is as defined in claim 1.

5. The compound according to claim 1, wherein m=1 and n=0 and the rings B and C together with the 2,6-naphthalene group form a group selected from the following formulae or their mirror images, wherein * denotes the linkage to the acetylene group: 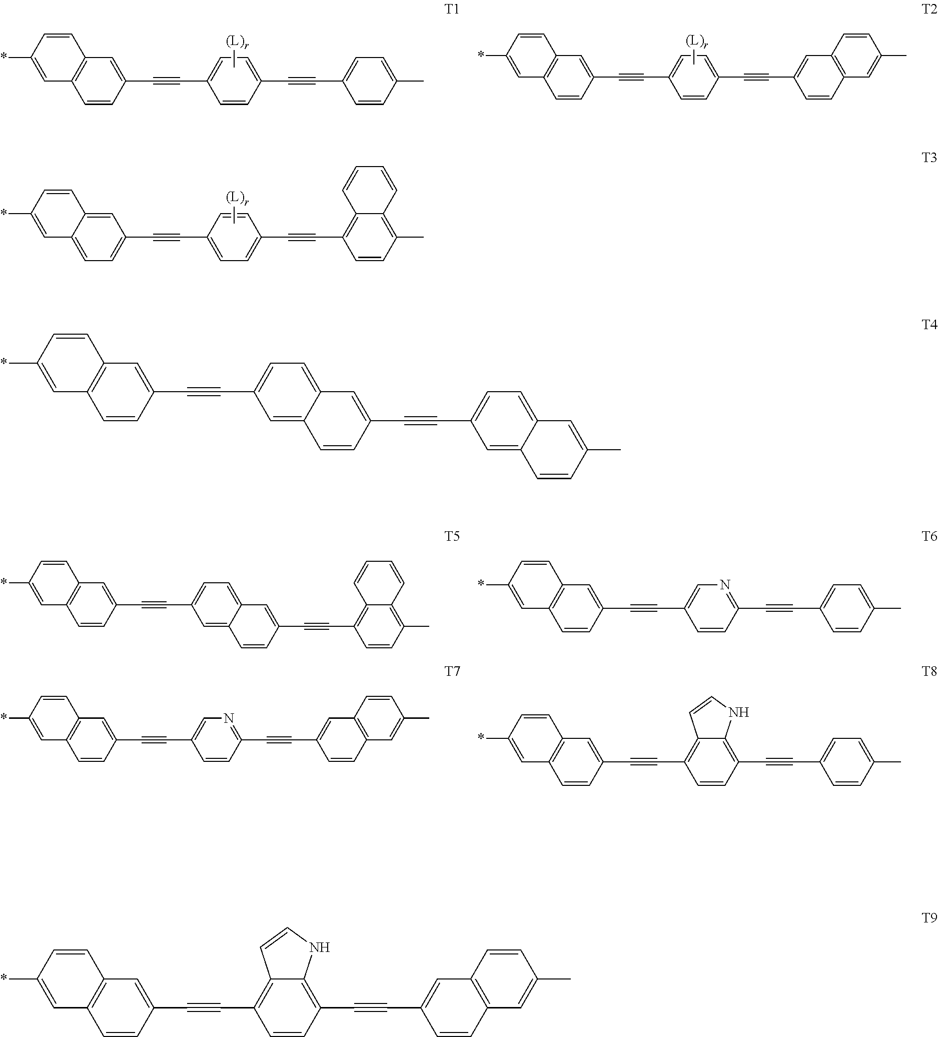
wherein the naphthalene groups are optionally substituted with one or two groups L, r is 0, 1, 2, 3 or 4, and L is as defined in claim 1.

6. The compound according to claim 1, wherein it is selected from the following subformulae: 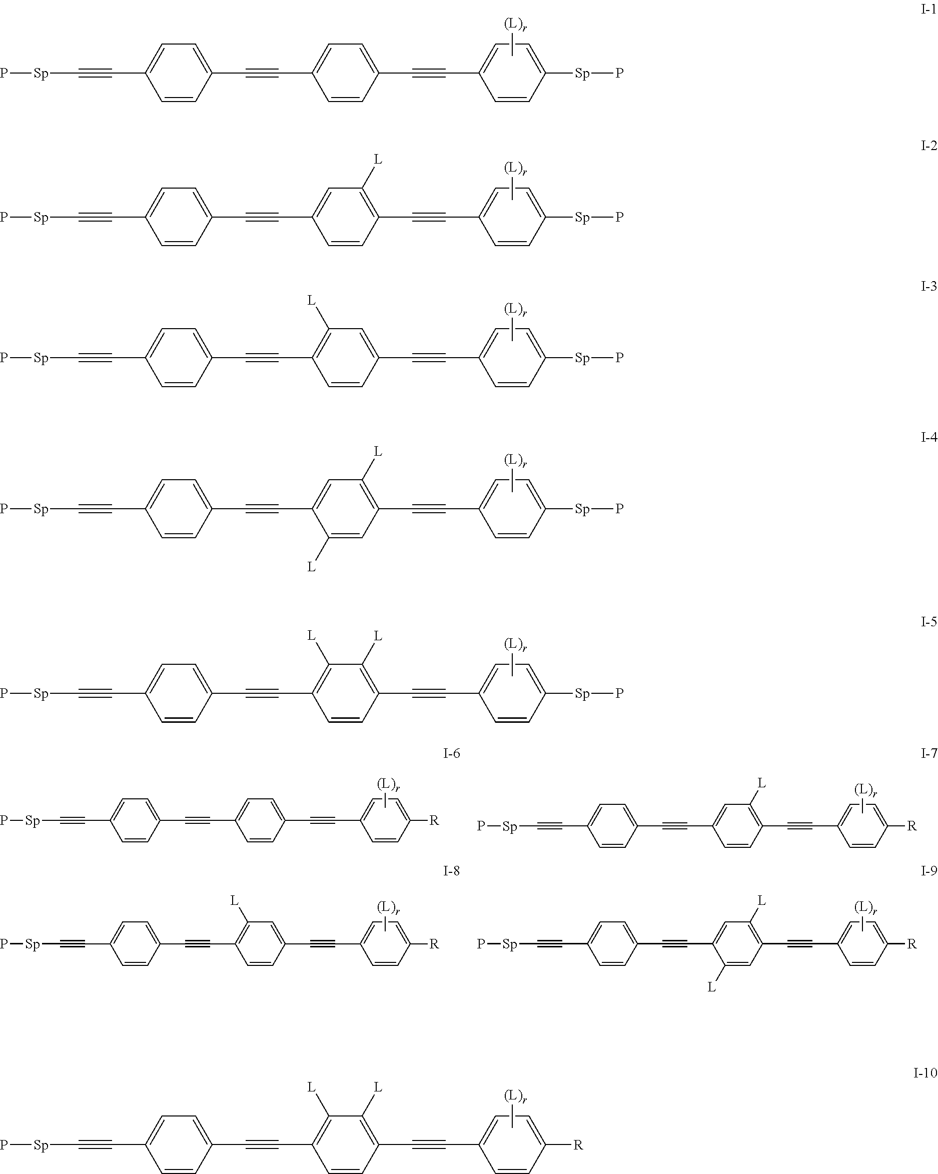 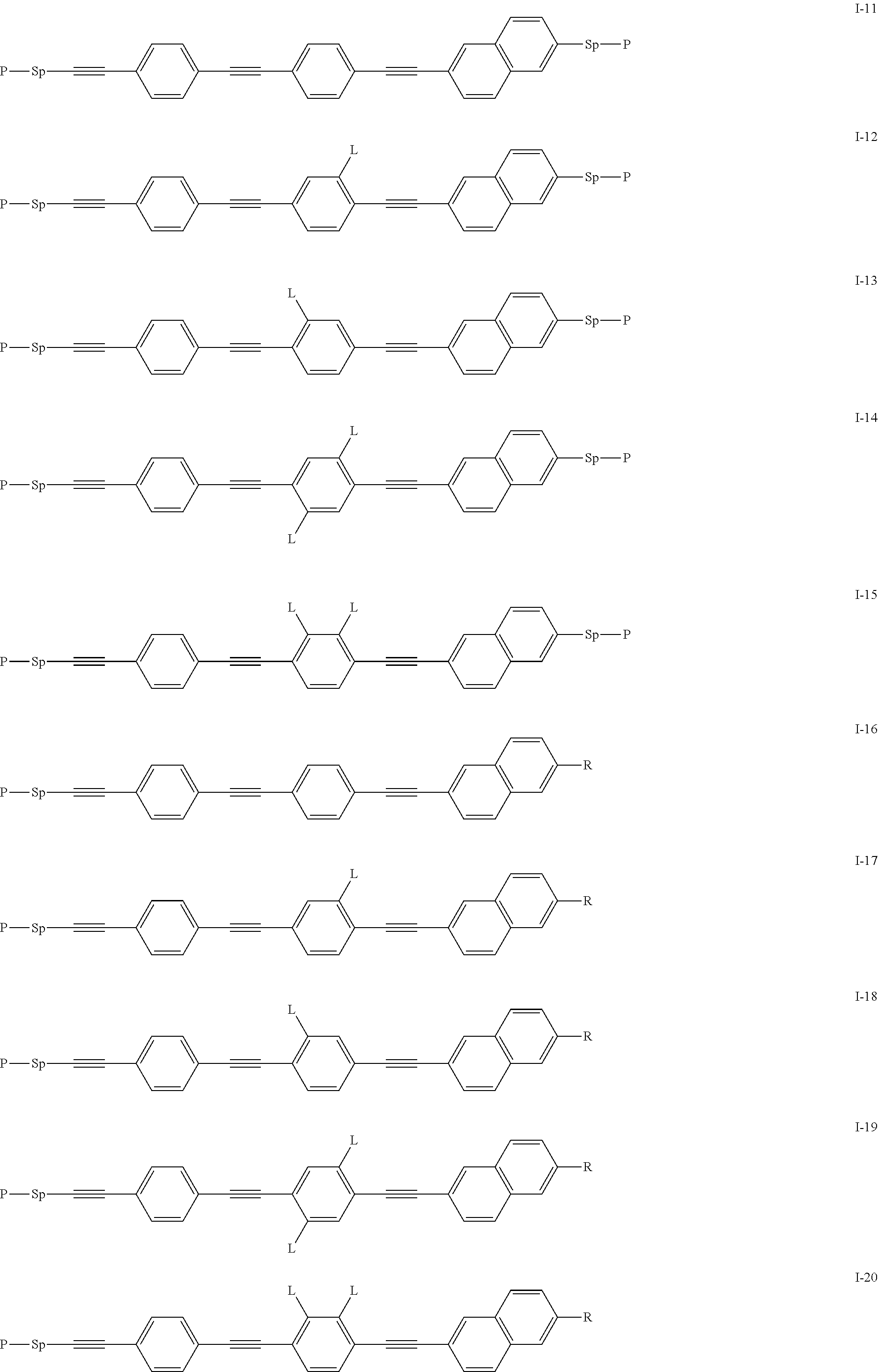 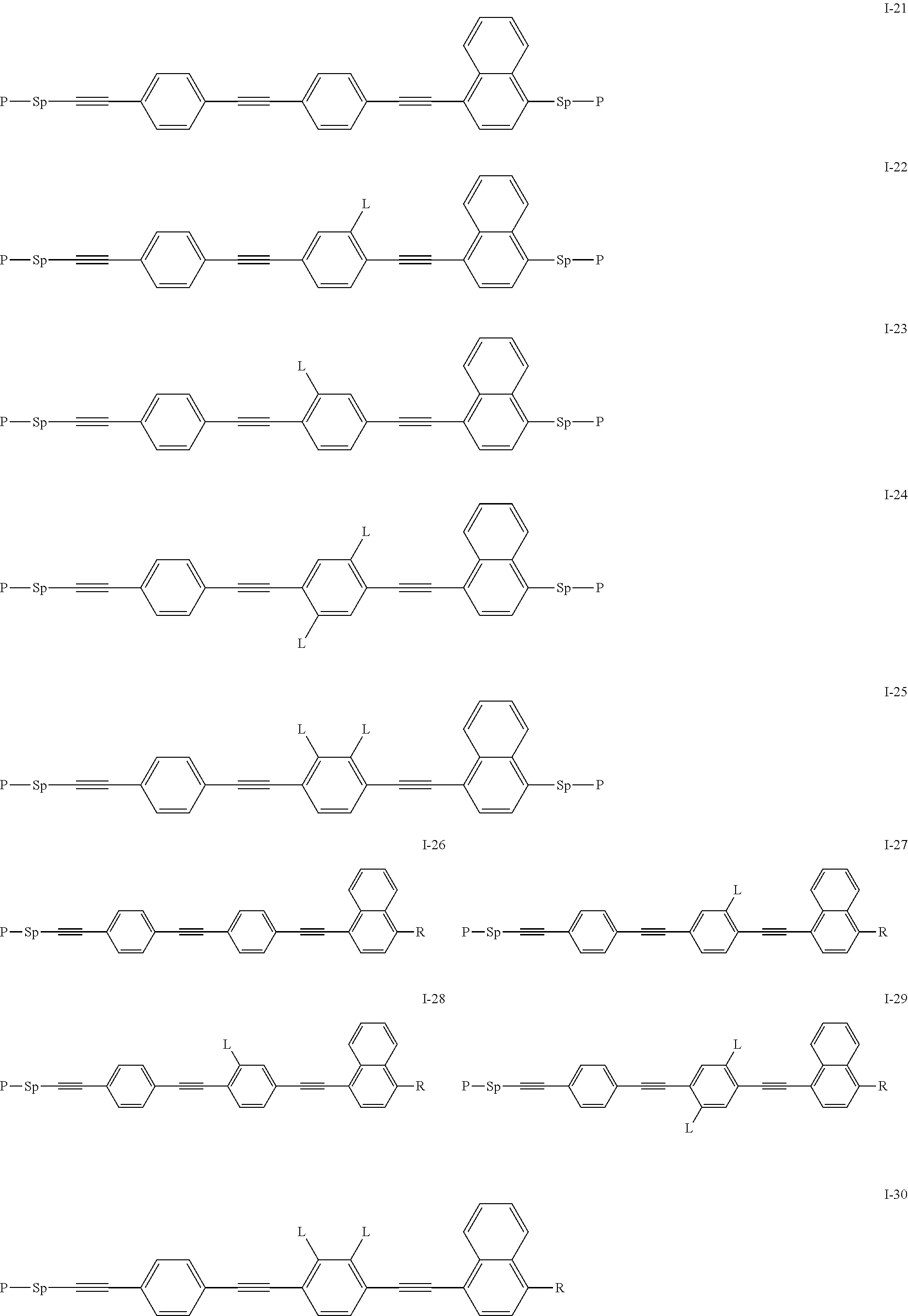 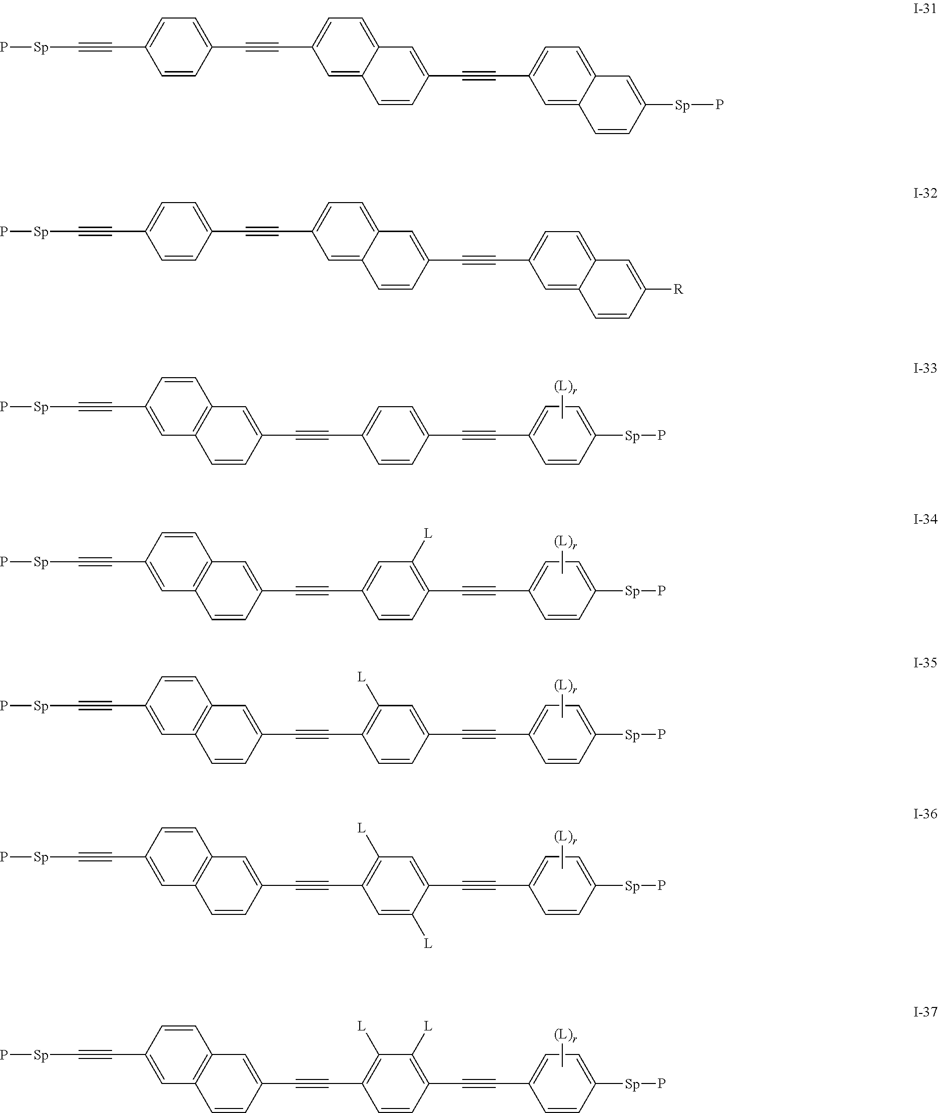 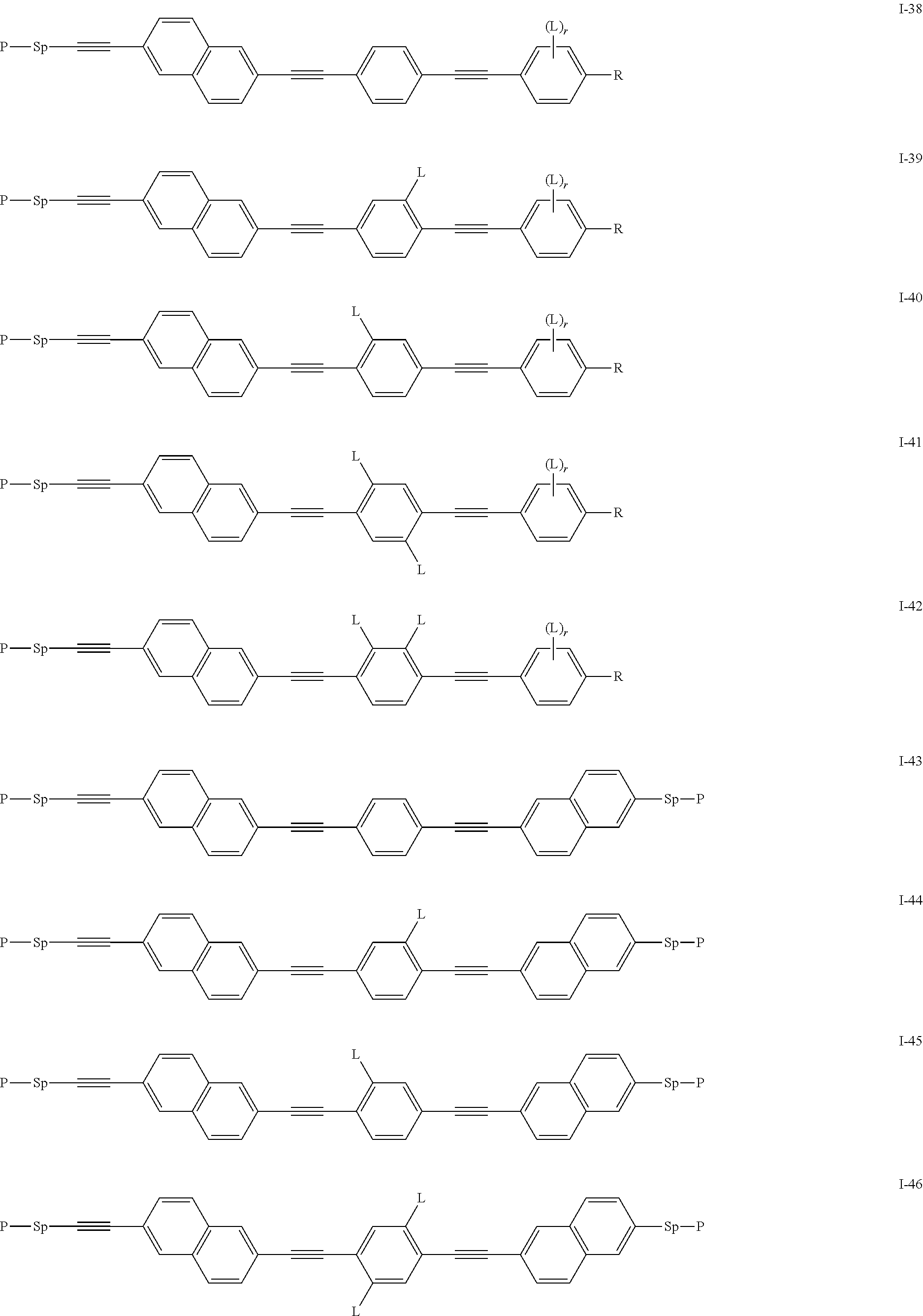 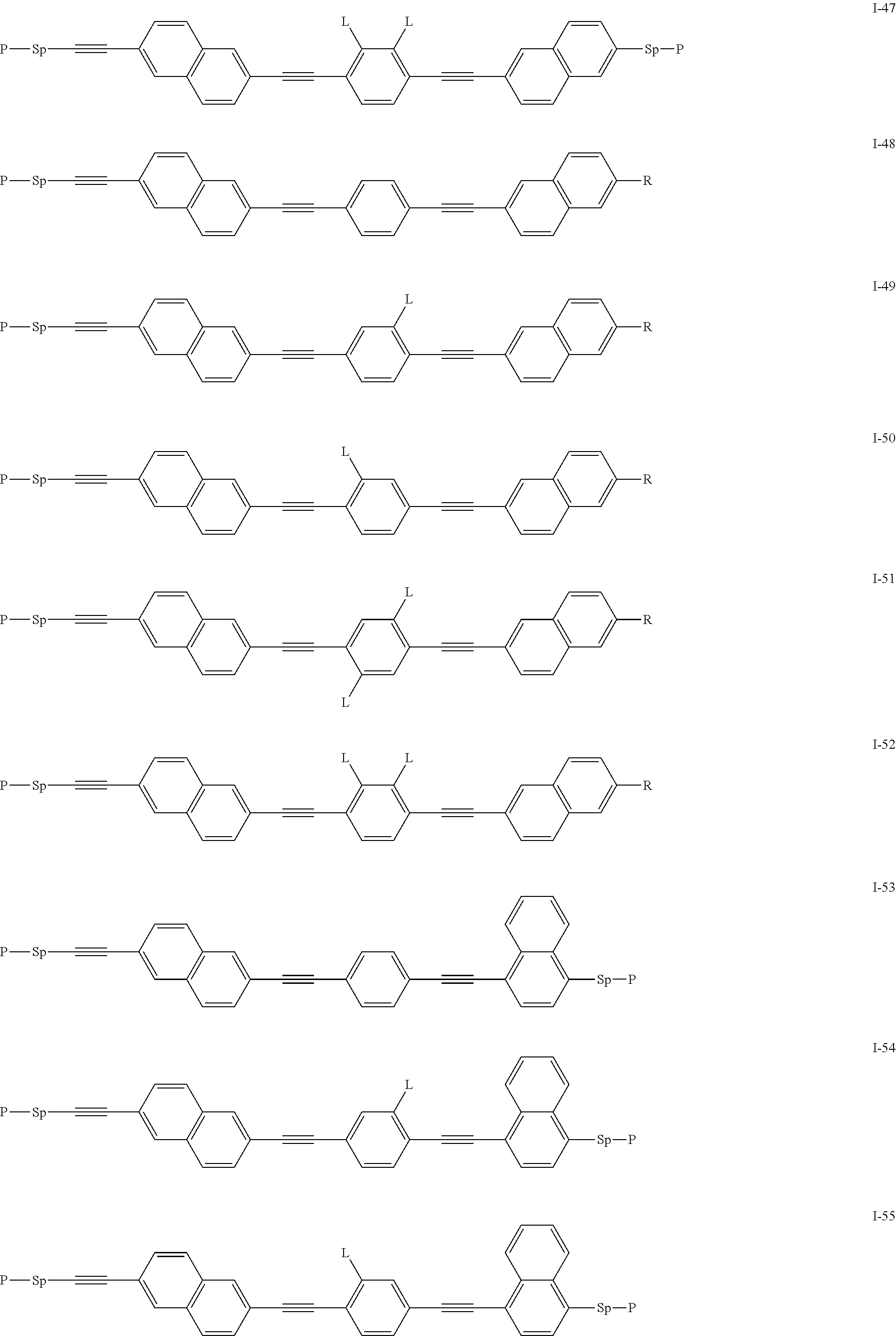 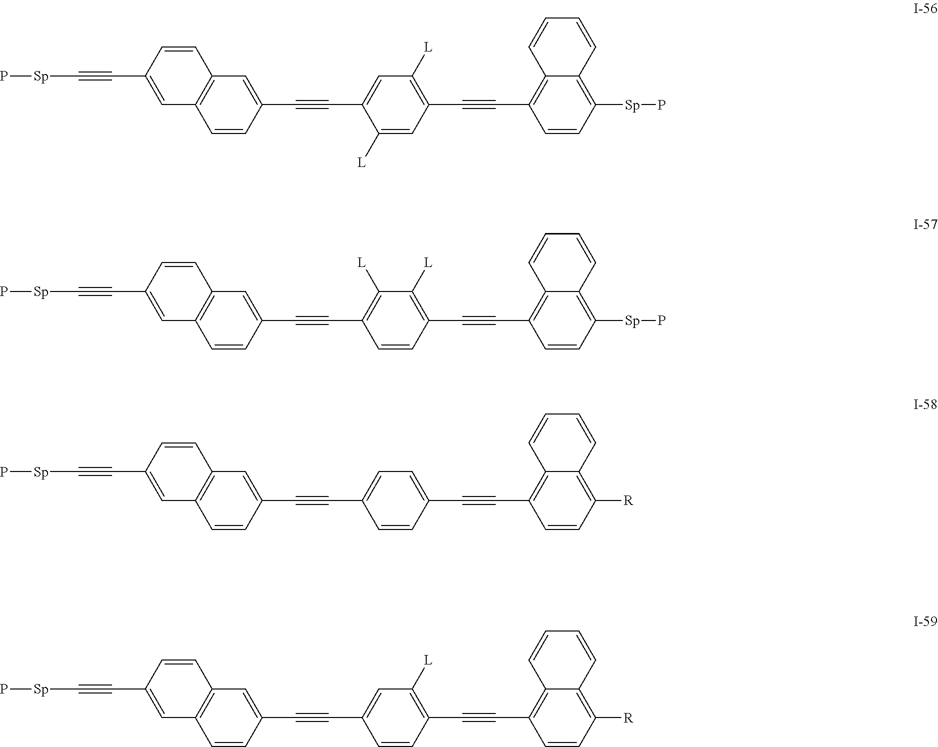 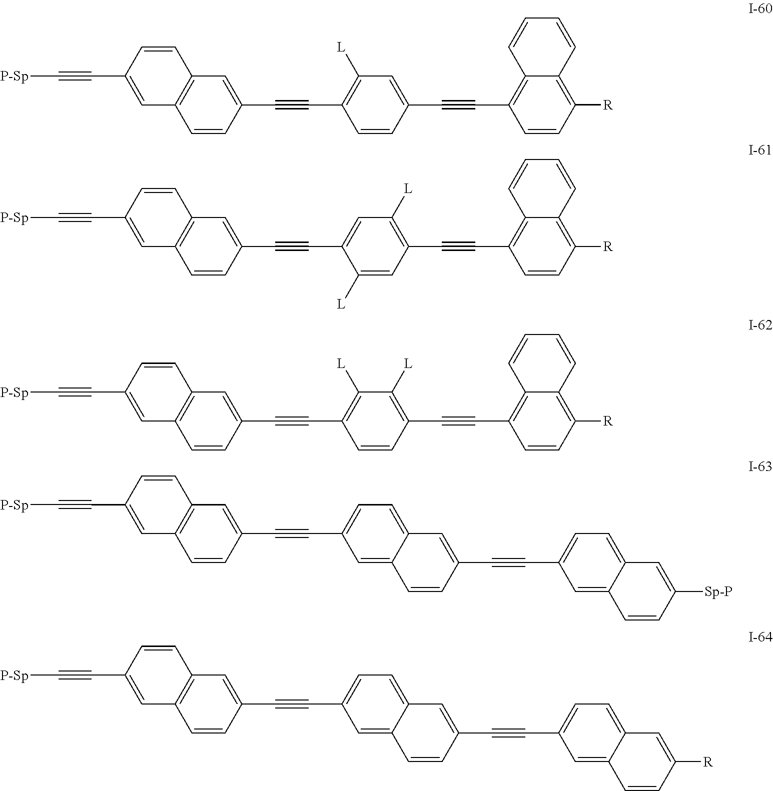
wherein the naphthalene groups are optionally substituted with one or two groups L, and P, Sp, L and r, independently of each other and on each occurrence identically or differently, and R has one of the meanings given for R¹¹ in claim 1.

7. A mixture comprising one or more compounds as defined in claim 1, and further comprising one or more reactive mesogens, which are different from formula I.

8. A mixture comprising one or more compounds as defined in claim 1, and further comprising one or more chiral compounds which are optionally polymerisable and/or isomerisable, and optionally further comprising one or more reactive mesogens, which are different from formula I.

9. A formulation comprising a compound according to claim 1 and further comprising one or more solvents and/or additives.

10. A polymer or polymer film obtainable by polymerising a compound according to claim 1.

11. (canceled)

12. An optical, electrooptical or electronic device or a component thereof, comprising a compound according to claim 1.

13. The component of claim 12, which is selected from optical retardation films, polarizers, optical compensators, reflective films, diffraction or surface gratings, Bragg polarization gratings (Bragg PG), polarization volume gratings (PVG), polarization volume holograms (PVH), Pancharatnam Berry (PB) gratings, furthermore nonmechanical beam steering elements, optical waveguides, optical couplers or combiners, polarization beam splitters, partial mirrors, reflective films, alignment layers, colour filters, antistatic protection sheets, electromagnetic interference protection sheets, lenses for light guides, focusing and optical effects, polarization controlled lenses, PB lenses and IR reflection films.

14. The device of claim 12, which is selected from liquid crystal displays, organic light emitting diodes, autostereoscopic 3D displays, see-through near-eye displays, AR/VR systems, goggles for AR/VR applications, switchable windows, spatial light modulators, optical data storage devices, optical sensors, holographic devices, spectrometers, optical telecommunication systems, polarimeters or front-/backlights.
